SEQUENCE LISTING (12 sequences)

SEQ ID NO: 1 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 2 - protein, 3 aa, Artificial Sequence
  Description of Artificial Sequence Synthetic peptide
EFM

SEQ ID NO: 3 - protein, 13 aa, Artificial Sequence
  Description of Artificial Sequence Synthetic peptide
EFGAGLVLGGQFM

SEQ ID NO: 4 - DNA, 37 bp, Artificial Sequence
  Description of Artificial Sequence Synthetic primer
cctgccatcaatgtggccatgcatgtgttcagaaagg

SEQ ID NO: 5 - DNA, 37 bp, Artificial Sequence
  Description of Artificial Sequence Synthetic primer
cctttctgaacacatgcatggccacattgatggcagg

SEQ ID NO: 6 - DNA, 36 bp, Artificial Sequence
  Description of Artificial Sequence Synthetic primer
ccagtgagtctggagagccgcatgggctcacaactg

SEQ ID NO: 7 - DNA, 36 bp, Artificial Sequence
  Description of Artificial Sequence Synthetic primer
cagttgtgagcccatgcggctctccagactcactgg

SEQ ID NO: 8 - DNA, 21 bp, Artificial Sequence
  Description of Artificial Sequence Synthetic primer
gcggccctacgggcaccggtg

SEQ ID NO: 9 - DNA, 21 bp, Artificial Sequence
  Description of Artificial Sequence Synthetic primer
gctcattccttgggattggtg

SEQ ID NO: 10 - DNA, 44 bp, Artificial Sequence
  Description of Artificial Sequence Synthetic primer
acaaaaaagcaggctccaccatgaagattctcctgctgtgtgtg

SEQ ID NO: 11 - DNA, 43 bp, Artificial Sequence
  Description of Artificial Sequence Synthetic primer
ttgtacaagaaagctgggttttccatgcggcttttcctgcggt

SEQ ID NO: 12 - protein, 6 aa, Artificial Sequence
  Description of Artificial Sequence Synthetic 6xHis tag
HHHHHH

(19) United States
(12) Patent Application Publication
(10) Pub. No.: US 2013/0203083 A1
(43) Pub. Date: Aug. 8, 2013

(54) SERUM CLUSTERIN LEVELS IN SYSTEMIC AMYLOIDOSIS FEATURING CARDIOMYOPATHY

(71) Applicants: Lawreen Connors, Woburn, MA (US); Michael J. Greene, Allston, MA (US)

(72) Inventors: Lawreen Connors, Woburn, MA (US); Michael J. Greene, Allston, MA (US)

(73) Assignee: TRUSTEES OF BOSTON UNIVERSITY, Boston, MA (US)

(21) Appl. No.: 13/578,294

(22) PCT Filed: Feb. 10, 2011
(86) PCT No.: PCT/US11/24296

§ 371 Date: Sep. 25, 2012

Related U.S. Application Data
(60) Provisional application No. 61/303,065, filed on Feb. 10, 2010.

(51) Int. Cl. G01N 33/68

(52) U.S. Cl. CPC G01N 33/6893
USPC 435/7.92; 436/501; 435/7.1; 422/69; 435/287.2

(57) ABSTRACT

The present invention relates generally to diagnostic methods, systems, assays and kits for identification of subjects with cardiac amyloid deposits, where a low level of clusterin protein in a peripheral fluid sample, e.g., a serum sample from the subject, indicates the subject likely has cardiac amyloid deposits. Other aspects relate to methods of treatment of diseases or disorders characterized by cardiac amyloid deposits and transthyretin (TTR) amyloidosis, and more particularly to methods of treatment of cardiac-related amyloidosis and cardiac amyloid deposits in subjects with familial transthyretin (TTR), senile systemic amyloidosis (SSA), or familial amyloidodic polyneuropathy (FAP), or immunoglobulin light chain (AL) amyloidosis. Other aspects relate to methods and compositions comprising clusterin (CLU) or a clusterin agent (e.g. an agonist of clusterin activity or a biologically active fragment or derivative thereof), and their use in methods to treat a disease or disorder characterized by transthyretin (TTR) amyloidosis, e.g. senile systemic amyloidosis (SSA) or familial amyloidodic polyneuropathy (FAP), and their use in methods to treat amyloidotic cardiomyopathy associated with transthyretin (TTR) amyloidosis.

### CROSS REFERENCE TO RELATED APPLICATIONS

This application claims priority under 35 U.S.C. 119(e) of U.S. Provisional Patent Application Ser. No. 61/303,065 filed on Feb. 10, 2010, the contents of which are incorporated herein by reference in their entirety.

### GOVERNMENT SUPPORT

The present application was made with Government Support under Grant No: AG031804 awarded by the National Institutes of Health (NIH). The Government of the United States has certain rights thereto.

### FIELD OF THE INVENTION

The present invention relates in the field of diagnostics, in particular to diagnostic methods, system and assays to identify subjects with cardiac amyloid deposits. The present invention also relates to methods of treatment of amyloidotic cardiomyopathy (CMP), as well as the treatment of diseases or disorders characterized by cardiac amyloid deposits, including but not limited to, familial transthyretin (TTR), senile systemic amyloidosis (SSA), or familial amyloidodic polyneuropathy (FAP), or familial amyloidotic cardiomyopathy (FAC), or immunoglobulin light chain (AL) amyloidosis.

### BACKGROUND OF THE INVENTION

Understanding the fundamental mechanisms of protein homeostasis and its disruption are important in determining the etiology of the systemic amyloid diseases. Molecular chaperones, including the extracellular protein clusterin, play a significant role in maintaining proteostasis and have a unique capacity to bind and stabilize non-native protein conformations, prevent aggregation, and keep proteins in a soluble folding-competent state. Clusterin is a plasma protein which shares functional characteristics with small heat shock proteins and is dysregulated in several protein misfolding diseases, cancers, and cardiac-related pathologies. In addition, clusterin is found co-deposited in amyloid-β plaques in brains of patients with Alzheimer's disease and appears to regulate the toxicity and solubility of Aβ.

Various diseases are characterized by the occurrence of misfolded and aggregated proteins. These so called protein aggregation diseases have heterogeneous etiologies, and include disorders such as Parkinson's disease, Huntington's disease, α1-antitrypsin deficiency, localized and systemic amyloidoses, and alcoholic as well as non-alcoholic steatohepatitis (ASH and NASH). Mallory bodies (MBs) are a prototype of cytoplasmic protein aggregates which occur in a variety of chronic toxic and metabolic liver disorders, including ASH and NASH. MBs are formed from abnormally folded keratins, mainly keratin 8, and a variety of stress proteins (Zatloukal et al. 2000, 2002). Biochemical and immunohistochemical analyses have revealed that MBs are highly ubiquitinated and consistently contain p62 (Zatloukal et al., 2002; Stumptner et al., 2002). These data indicate that liver diseases featuring MBs may be due to improperly assembled keratins that are recognized as misfolded proteins and as such, a major target of the stress response.

Protein aggregation diseases are characterized by the occurrence of abnormal proteins, which aggregate intracellularly or in the extracellular space. Misfolding of proteins can be the result of structural modifications such as those that arise from oxidative cell injury or mutation. Oxidative attack on proteins may lead to amino acid modification and/or fragmentation which results in a loss of native protein secondary structure; affected proteins may become unfolded or adopt conformations with exposed non-polar residues. Such structural rearrangements favor aggregation through hydrophobic interactions and cross-linking reactions at these amino acids (Grune et al., 1997).

Currently, there are no lasting cures for diseases caused by misfolded or aggregated proteins and therapeutic strategies are quite varied. Moreover, prophylaxis of such disorders is often impossible due to lack of suitable early stage markers. Moreover, the side effects connected with prophylactic administration of certain treatments for these disorders might be contra-indicated, especially for low or non-high risk patients.

Clusterin (apolipoprotein J) is a sulfated, heterodimeric glycoprotein containing two, 40 kDa chains joined by a unique five disulfide bond motif. Encoded on a 2-kb mRNA, clusterin is transcribed from a single copy gene located on chromosome 14 in mice and chromosome 8 in humans. Clusterin contains several domains including amphipathic helical, heparin-binding, and lipid-binding regions. This protein was initially identified from ram rete testes fluid and named for its ability to elicit clustering of Sertoli cells supporting sperm maturation and development (NCBI/GenBank Accession No. NM_{—}203339, NM_{—}001831) Thereafter, species homologues have been isolated and cloned by a number of groups working in widely divergent areas, resulting in a number of synonyms for clusterin including testosterone repressed prostate message-2 (TRPM-2), sulfated glycoprotein-2 (SGP-2), apolipoprotein J, SP-40, 40, complement cytolysis inhibitor (CLI), dimeric acidic glycoprotein (DAG), gp 80, NA1/NA2, glycoprotein III, etc. Clusterin circulates in blood partially associated with the high density lipoprotein (HDL) fractions; thus, it is considered a component of HDL forming a complex together with apolipoprotein AI and paraoxonase (NCBI Accession No. NM_{—}000446). Paraoxonase is reportedly an enzyme with strong antioxidant properties. Clusterin and these serum-associated proteins are present at high levels in the lesions of patients with atherosclerosis. Clusterin is translated with a hydrophobic signal peptide that is 21 amino acids in length.

Reported functions of clusterin include apoptosis regulation, complement defense, lipid recycling, membrane protection, and maintenance of cell-cell or cell-substratum contacts. Clusterin can effectively bind to lipids including both cholesterol and oxysterols, and has been shown to promote efflux of cholesterol and oxysterols from lipid-laden foam cells. This protein can also inhibit complement-mediated cell death, and promote cell aggregation and adhesion. Clusterin has been found to be an anti-apoptotic protein. It has been reported that clusterin expression is induced and confers resistance to apoptotic cell death induced by heat shock and oxidative stress. High levels of clusterin have been shown in tissues with apoptosis.

Developmental regulation of clusterin expression has been reported in many tissues including the heart, kidney, lung, and brain. In the heart, clusterin is found in both the atria and ventricles of the fetal mouse heart, but in the adult heart, only the atria show positive stains for clusterin. However, marked induction of clusterin can be detectable in the heart with acute infarction, in particular the peri-infarct zone. Induction of clusterin is also observed in the myocardium with inflammation suggesting a possible protective role for the protein in cardiac tissue.

Clusterin-deficient mice appear to be more prone to developing myocarditis compared to age- and sex-matched wild-type controls. In the kidney, clusterin is expressed in the ureteric bud but not in surrounding mesenchyme. When the mesenchyme is induced to differentiate into renal epithelium, clusterin expression takes place and continues in developing tubules. In newborn mice, almost all the tubules express clusterin, but adult tubules rarely express clusterin. Similar to the time course in the heart and kidney, developing fetal tissue but not adult lung contains clusterin.

Clusterin mRNA and protein are expressed at high levels in the heart, a tissue that is completely resistant to the cytotoxic effects of hyperthermia. A similar finding was reported in the developing brain. Clusterin expression occurs in the earliest neurons of the cortical plate on embryonic day (E) 12 and continues to increase in an age-dependent manner, with the greatest intensity of expression in the postnatal mature brain. Clusterin is also frequently found in neuronal degenerative disorders, such as Alzheimer's disease.

Human transthyretin (TTR) is a secreted, thyroid-hormone binding protein produced in the liver and the choroid plexus of the brain. TTR is found in the serum and cerebrospinal fluid as a tetramer composed of four identical non-covalently associated subunits W. In certain individuals, TTR is a human disease-associated amyloidogenic protein that undergoes conformational changes leading to self-aggregation and assembly into highly ordered amyloid fibrils [2,3]. Two types of TTR-associated amyloidosis have been reported: senile systemic amyloidosis (SSA), which affects approximately 25% of the population over 80 years of age, and familial amyloid polyneuropathy (FAP), which affects approximately 1 in 100,000 persons [4]. Wild-type TTR deposition has been linked to the sporadic amyloid disease, SSA. In contrast, hereditary amyloidosis linked to TTR mutations (ATTR) is caused by the aggregation of one of over 100 TTR variants; depending on the clinical phenotype, ATTR is also called familial amyloid polyneuropathy, familial amyloid cardiomyopathy, and familial central nervous system amyloidosis. FAP typically presents in the peripheral tissues, leading to neurodegeneration and/or organ disruption [4]. In familial TTR-associated amyloidosis, V30M was the first variant linked to FAP and described in a Portuguese population. This variant is arguably the most common pathologic TTR (as TTR-V 122I is present in 4% of the African American population and linked to cardiac amyloid disease). In terms of native conformational stability, TTR-V30M has been shown to exhibit intermediate stability compared to the highly unstable TTR-L55P, the most lethal naturally occurring amyloidogenic variant described to date [5].

At the present time, the only effective treatment for familial TTR amyloidosis is orthotopic liver transplantation [6]. Since liver transplantation has a number of limitations, including a shortage of donors and high cost, this strategy does not provide practical means of treating FAP patients. Among many efforts to develop alternative therapies for familial amyloidosis, one possible therapeutic strategy may be to use small molecules that stabilize the circulating TTR tetramer, thereby reducing the amount of monomer available to form amyloid fibrils [7-9]. Diflunisal and Fx-1006A have been used for TTR tetramer stabilization and are currently in Phase II/III clinical trials. However, small molecule-mediated TTR stabilization still has the possibility of interfering with other biological processes in vivo, so other safe and promising therapeutic approaches are being strenuously pursued.

Cardiac amyloidosis is a disorder caused by extracellular deposits of amyloid proteins in the myocardium, e.g., amyloid fibrils composed of TTR (mutant and wild-type forms) or immunoglobulin light chain deposit in the heart tissue causing cardiac dysfunction and eventually heart failure. Cardiac amyloidosis, also referred to as amyloidotic cardiomyopathy (CMP), can be difficult to diagnose because the signs can be related to a number of different conditions. Moreover, determination of the type of cardiac amyloidosis, i.e. identification of the amyloid fibril protein is critical as prognosis and management strategies vary. In TTR-associated cardiac amyloidosis, the disease can be a long-term chronic condition that progresses unabated with worsening manifestations of disrupted organ function. Current diagnosis of amyloidotic cardiomyopathy is complex and relies on expensive scans utilizing specialized imaging equipment in addition to a variety of histopathological, immunological, and biochemical testing procedures; there is no single test that easily identifies a subject with cardiac amyloidosis. Accordingly, there is need in the art for methods to easily diagnose a subject with amyloidotic cardiomyopathy so that the subject can be managed properly and treated with an appropriate regimen to relieve symptoms of amyloidotic cardiomyopathy or to prevent deterioration of the subject's condition.

### SUMMARY OF THE INVENTION

One aspect of the present invention relates to methods, systems and kits for determining if a subject has amyloidotic cardiomyopathy (CMP) or cardiac amyloid deposits, the method comprising measuring the level of clusterin protein in a biological sample from the subject, e.g., a fluid sample from the subject, for example, a blood or serum sample, where a low level of clusterin protein by a statistically significant level as compared to a reference level of clusterin level is indicative of the subject having cardiac amyloidosis.

Another aspect of the present invention provides an additional step of treating a subject identified as having cardiac amyloid deposits, and/or amyloidotic cardiomyopathy (CMP) by the methods and systems as disclosed herein, where the treating can comprise administering to the subject a pharmaceutical composition for treatment of disorders characterized by amyloidosis in the heart and cardiac-related amyloidosis.

Accordingly, one aspect of the present invention relates to a method for assessing a subject at risk of having cardiac amyloid deposits, the method comprising measuring the level of clusterin protein in a biological sample obtained from the subject, wherein a decrease in the level of clusterin protein in the biological sample by a statistically significant amount as compared to a reference level of clusterin protein is indicative of the subject being at risk of having cardiac amyloid deposits. In some embodiments, if the level of the clusterin protein in the biological sample is decreased by more than 40%, or more than 50% or more than 60% as compared to a reference level of clusterin protein, it is indicative of the subject being at risk of having cardiac amyloid deposits.

In some embodiments, a level of the clusterin protein of less than about 0.5 mg/mL, or less than about 0.4 mg/mL, or less than about 0.3 mg/mL, or less than about 0.1 mg/mL, or between about 0.4 mg/mL-0.01 mg/mL in a biological sample, e.g., serum sample, is indicative of the subject being at risk of having cardiac amyloid deposits.

In some embodiments, the subject is a mammalian subject, for example a human subject. In some embodiments, the biological sample can be any peripheral fluid sample obtained from the subject, and can be a blood sample, plasma sample or a serum sample.

Typically, in some embodiments, the level of clusterin protein is measured by a protein binding molecule, for example but not limited to, an antibody, or an antibody fragment or a ligand-binding partner of clusterin, or functional fragments, thereof. In some embodiments, the level of clusterin protein is measured by immunoassay, for example an ELISA; however, other methods to measure clusterin protein levels are encompassed in the methods and systems as disclosed herein.

In some embodiments, a subject who is assessed for having cardiac amyloid deposits is a subject who has, or is at risk of developing an amyloid disease, e.g., a disease or disorder associated with amyloidosis. In some embodiments, a disease or disorder associated with amyloidosis is a disease or disorder associated with transthyretin (TTR) amyloidosis, for example but not limited to senile systemic amyloidosis (SAA), familial amyloid neuropathy (FAP), familial amyloid cardiomyopathy (FAC), familial central nervous system amyloidosis. In alternative embodiments, a disease or disorder associated with amyloidosis is immunoglobulin light chain (LC) amyloidosis.

In some embodiments, a reference level of clusterin, e.g., clusterin protein is the level of clusterin, e.g., clusterin protein present in the same type of biological sample obtained from a healthy subject or a subject not having a disease or disorder associated with amyloidosis, or a subject not having cardiomyopathy.

In some embodiments, a subject identified to have, or likely to have cardiac amyloid deposits using the methods and systems as disclosed herein is at risk of having, or has congestive heart failure, arrhythmias or progressive amyloid heart failure.

In some embodiments, the method also encompasses administering to a subject identified to be at risk of having amyloid deposits using the methods and systems as disclosed herein a composition comprising a clusterin protein or a functional fragment having clusterin activity, or an agonist of clusterin activity.

Accordingly, the present invention relates to use of clusterin levels in a fluid sample from the subject, e.g., from a blood or serum sample, as a biomarker to diagnose cardiac amyloidosis. As such, the present invention provides a non-invasive method to identify a subject with cardiac amyloidosis and amyloidotic cardiomyopathy (CMP). Additionally, the present invention relates to methods, systems and kits which can be used to determine specific cardiac amyloidosis disease types, e.g., to distinguish a subject having AL vs. ATTR vs. SSA cardiac amyloidosis. For example, as demonstrated in Example 3 herein, subjects with SSA-CMP have the lowest serum clusterin levels, followed by AL-CMP subjects which have the next lowest serum clusterin levels, followed by ATTR-CMP, which have the next lowest serum clusterin levels. For example, in one exemplary example, a subject with SSA-CMP can have a serum clusterin level of about 0.2 mg/ml or lower, and a subject with AL-CMP can have a clusterin serum level in the range of about 0.25-0.34 mg/ml, and a subject with ATTR-CMP can have a serum clusterin level of about 0.35-0.5 mg/ml. Accordingly, the present invention can be used to distinguish which type of amyloid disease the subject has.

Other aspects of the present invention relate to use of the methods, systems and kits as disclosed herein to monitor disease progression, for example, where a clusterin level is measured in a subject at a first timepoint and then at subsequent timepoints, and the clusterin level measured at subsequent timepoints can be compared to one or more clusterin levels measured at one or more earlier timepoints. In such embodiments, a decrease in the clusterin level as compared to the earlier timepoint will indicate an increased disease progression, whereas if the clusterin level remains the same it can indicate a halting or attenuation of the disease progression. In some embodiments, if a clusterin level measured at a later timepoint has an increased as compared to a previous measured clusterin level from the subject, it can indicate that the subject has reduced cardiac amyloid deposits and improved prognosis.

Similarly, another aspect of the present invention relate to use of the methods, systems and kits as disclosed herein as a prognostic indicator and to monitor treatment outcome, for example, where a clusterin level is measured in a subject at a first timepoint prior to treatment, and then at subsequent timepoints after or during treatment, and the clusterin level measured at subsequent timepoints (e.g., after treatment) can be compared to one or more clusterin levels measured at an earlier timepoint (prior to treatment). In such embodiments, an increase in the clusterin level as compared to the earlier timepoint will indicate a good prognosis and effective treatment, whereas if the clusterin level remains the same it can indicate a halting or attenuation of the disease progression. In some embodiments, if a clusterin level measured at a later timepoint (e.g., after or during treatment) has decreased as compared to a previous measured clusterin level (e.g., prior to treatment), it can indicate that the treatment is not effective and optionally, a physician can change one or more of the treatment, dose, regimen, administration route etc.

Accordingly, another aspect of the present invention provides pharmaceutical compositions and methods for treatment of disorders characterized by amyloidosis in the heart and cardiac-related amyloidosis.

One aspect of the invention relates to a method for the treatment or prevention of a disease or disorder associated with transthyretin (TTR) amyloidosis in a subject, the method comprising administering to the subject a composition comprising a clusterin protein, or a functional variant having clusterin activity, or an agonist of clusterin activity, wherein the clusterin protein or agonist of clusterin activity (e.g. a clusterin agent) decreases the amount of TTR amyloid fibrils in the subject.

In one embodiment, the clusterin protein is human clusterin protein, for example, a clusterin protein is SEQ ID NO: 1 or a functional variant or functional fragment or functional mutein thereof. In some embodiments, an agonist of clusterin activity, which is also referred herein as a “clusterin agent” is a functional isoform, a functional mutein, a functional fused protein, a functional derivative, a functional variant or a functional fragment of clusterin protein. In some embodiments, a functional variant or functional mutein is a variant or mutein of SEQ ID NO: 1. In some embodiments, a variant or mutein has at least 80%, or at least 85%, or at least 90%, or at least 95% homology with SEQ ID NO: 1.

In some embodiments, a disease or disorder associated with transthyretin (TTR) amyloidosis is selected from the group consisting of: senile systemic amyloidosis (SAA), familial amyloid neuropathy (FAP), familial amyloid cardiomyopathy (FAC), familial central nervous system amyloidosis.

In some embodiments, a composition comprising clusterin is at an acidic pH, for example where the pH is less than pH 6.0, such as where the pH is between pH 6.0 and pH 4.5.

In some embodiments, a subject is a mammalian subject, such as a human subject.

Another aspect of the present invention relates to a method for the diagnosis of a subject with cardiac amyloid deposits, the method comprising measuring the level of clusterin protein in a biological sample obtained from the subject, wherein if the level of clusterin protein in the biological sample from the subject is lower by a statistically significant amount relative to a reference level of clusterin protein, the subject likely is at risk of having cardiac amyloid deposits.

Another aspect of the present invention relates to a method comprising administering to a subject for the treatment of disease or disorder associated with amyloidosis an effective amount of a clusterin protein, or a functional variant having clusterin activity, or an agonist of clusterin activity indicated by the presence of a decreased amount of clusterin protein in a biological sample obtained from the subject by a statistically significant amount relative to the level of clusterin protein present in a reference biological sample. In some embodiments, a subject is has, or is at risk of having a disease or disorder associated with amyloidosis, such as, for example, transthyretin (TTR) amyloidosis, including but not limited to, senile systemic amyloidosis (SAA), familial amyloid neuropathy (FAP), familial amyloid cardiomyopathy (FAC), familial central nervous system amyloidosis. In some embodiments, a disease or disorder associated with amyloidosis is immunoglobulin light chain (LC) amyloidosis.

In some embodiments, a subject is a human subject. In some embodiments, the biological sample is a blood sample, plasma sample or a serum sample. In some embodiments, the level of clusterin protein is measured by a protein binding molecule, such as, for example but not limited to a protein binding molecule is an antibody, or an antibody fragment or a ligand binding partner of clusterin, or functional fragments thereof. In some embodiments, the level of clusterin protein is measured by immunoassay, such as by an ELISA.

In some embodiments, a reference level of clusterin is the level of clusterin present in the same type of biological sample obtained from a healthy subject or a subject not having a disease or disorder associated with amyloidosis.

Another aspect of the present invention relates to a system for assessing if a subject has cardiac amyloid deposits, the system comprising: (a) a determination module configured to receive a biological sample, measure levels of clusterin protein in the biological sample and to output information of the level of clusterin protein in the biological sample; (b) a storage device configured to store clusterin level output information from the determination module; (c) a comparison module adapted to receive input from the storage device and compare the data stored on the storage device with at least one reference clusterin level data, wherein if the reference clusterin level data is 1.5-fold or more higher than the input clusterin protein level information, the comparison module provides information to an output module that the biological sample is associated with a subject that deviates from the reference clusterin protein level; and (d) an output module for displaying the information to the user.

### BRIEF DESCRIPTION OF FIGURES

FIGS. 1A-1B show an illustration as previously reported in Lee et al., that acidic conditions enhance the binding of clusterin to wt-TTR and the variants V30M and L55P TTR. FIG. 1A shows representative immunoblots of clusterin after equal amounts of purified GST, GST-fusion wt-TTR, and GST-fusion TTR variants were incubated with purified recombinant clusterin in PBS or acetate buffer (pH 4.4) for 12 h. FIG. 1B is a similar immunoblot as shown in FIG. 1A, but where samples were incubated in PBS or acetate buffer (pH 4.4) for 24 h. Bar graphs show normalized clusterin abundance observed 24-hr incubation. The density of bands was analyzed with Sigmagel. P, PBS; A, acetate buffer. Error bar=se. *P<0.05.

FIGS. 2A-2B is an illustration as previously reported in Lee et al., showing that clusterin inhibits amyloid fibril formation of the variants V30M and L55P TTR. FIG. 2A shows amyloid fibril formation of wt-TTR and TTR variants. About 0.22 mg/ml or 0.44 mg/ml of wt-TTR and TTR variants was incubated in acetate buffer (pH 4.4) at 37° C. for times indicated. The extent of amyloid fibril formation was monitored by turbidity at 450 nm. FIG. 2B shows that 0.22 mg/ml of TTR proteins which were incubated in the presence or absence of clusterin at 37° C. for 1 day and the extent of amyloid fibril formation was determined by turbidity at 450 nm. DATA points represent means of at least three independent experiments; error bar=se. *P<0.05

FIGS. 3A-3C is an illustration as previously reported in Lee et al., showing that clusterin stabilizes tetrameric and dimeric forms of TTR proteins. FIG. 3A shows 0.22 mg/ml of serum TTR proteins is incubated with 0.15 mg/ml of clusterin at 37° C. in acetate buffer (pH 4.4) for the times indicated, then TTR proteins were cross-linked and analyzed by Western blot. FIG. 3B shows 0.22 mg/ml of wt-TTR and TTR variants purified from E. coli were incubated with 0.15 mg/ml of clusterin at 37° C. for 3 days at, pH 4.4. The proteins were cross-linked and analyzed by Western blot analysis. Uncross-linked samples were used as a quantitative control. Results of two independent experiments (Exp. 1 and Exp. 2) are shown. FIG. 3C is similar to the western blot in FIG. 3B, showing samples that were incubated at pH 5.0 for times indicated. TTR proteins in PBS were used as a control for TTR tetramer. P, PBS; 1D, 1 day, 3D, 3 days, 6D, 6 days.

FIG. 4 is an illustration as previously reported in Lee et al., which shows clusterin suppresses TTR fibril-induced cell toxicity. Fibrils of wt-TTR and the variants V30M and L55P TTR were made by incubating TTR solutions in the presence or absence of clusterin at room temperature for 1 day, and pelleting fibrils at 15,000 rpm for 60 min. The pellet fraction of wt-TTR and the variants V30M and L55P TTR fibrils were dissolved with culture media and then used to treat SH-SY5Y cells for 48 h. Cell viability was measured by MTT assay. Data points are expressed as percentage of soluble forms of TTR-treated groups. Bar graphs represent means of three independent experiments; error bar=se. *P<0.05.

FIG. 5 shows clusterin concentrations in sera of patients ≧65 years diagnosed with senile systemic amyloidosis (SSA, n=35), hereditary transthyretin amyloidosis (ATTR, n=19), immunoglobulin light chain amyloidosis (AL) with renal involvement (n=20) or cardiomyopathy (CMP, n=15), and healthy controls (n=25). P-value ≦0.05 (*, #); 0.005 (**, ##); 0.0005 (***, ###). Significance was determined using a one-way ANOVA (p≦0.0001) and Tukey's multiple comparison test.

FIG. 6A-6C show receiver operating characteristics (ROC) curves and analysis of serum clusterin (CLU) levels in controls vs. SSA-CMP.

FIGS. 7A-7C show ROC analysis of serum CLU levels in controls vs AL-CMP.

FIGS. 8A-8C show ROC analysis of serum CLU levels in SSA-CMP vs AL-no CMP.

FIGS. 9A-9C show ROC analysis of serum CLU levels in AL-CMP vs AL-no CMP.

FIGS. 10A-10M show light microscopic images of immunohistochemical staining for amyloid precursor proteins transthyretin (TTR) and Ig light chain (LC) and for the extracellular protein clusterin (CLU) in serial sections of control (no amyloid), senile systemic amyloidosis (SSA), familial TTR amyloidosis (ATTR), and LC amyloidosis (AL) cardiac tissue specimens. No staining was observed for TTR (FIG. 10A) or LC (FIG. 10E) in the control tissue, though subtle staining for CLU (FIG. 10I) was present. SSA and ATTR sections showed extensive pericellular staining for TTR (FIG. 10B, FIG. 10C) and CLU (FIG. 10J, FIG. 10K); there was no evidence of staining with LC (FIG. 10F, FIG. 10G). In the AL sections, abundant amounts of LC were observed (FIG. 10H) and strong CLU staining (FIG. 10L) was noted; there was no staining for TTR (FIG. 10D). Cardiac tissues from 3 cases in each amyloid group (SSA, ATTR, AL) were analyzed and consistent results were obtained. Original magnification, ×40. Scale bars=50 microns, FIG. 10M shows immunoblot analysis of protein extracts from other portions of the same cardiac samples (control, SSA, ATTR, and AL) analyzed in the immunohistochemical studies.

FIGS. 11A-11H show electron microscopy images of immunogold staining for amyloid precursor proteins, TTR and LC, and CLU in cardiac tissue specimens from a control and cases of SSA, ATTR, and AL amyloidosis. Control (FIG. 11A, FIG. 11B), SSA (FIG. 11C, FIG. 11D), ATTR (FIG. 11E, FIG. 11F), and AL (FIG. 11G, FIG. 11H) cardiac tissues were labeled for TTR or LC (column 1) and CLU (column 2) using antibodies conjugated to 10 nm electron dense gold particles. No amyloid deposits were visible in control sample, no immunogold labeling for TTR, LC or CLU was detected. In SSA, ATTR and AL specimens, amyloid deposits (arrows) were present and located adjacent to cardiomyocytes. Extensive gold particle immunolabeling (arrowheads) for TTR was evident in SSA (FIG. 11C) and ATTR (FIG. 11E) samples, and abundant staining was detected in the AL sample (FIG. 11G). Immunogold staining for CLU (clusterin) demonstrated that clusterin was present are localized to amyloid deposits in SSA (FIG. 11D), ATTR (FIG. 11F), and AL (FIG. 11H). Images are ×80,000 magnification.

FIGS. 12A-12D show serum concentrations of CLU in patients with cardiomyopathy (CMP) and healthy controls. FIG. 12A shows serum levels of CLU quantified by ELISA in normal age-matched controls (white bar), patients with AL or ATTR but no CMP and patients with CMP due to other causes (gray bars), and patients with AL, ATTR, and SSA CMP (black bars). FIG. 12B shows immunoblot analysis of representative sera from each group showing the decreased amounts of CLU in the groups with CMP and amyloidosis. FIG. 12C show retrospective analysis of circulating BNP concentrations from matched serum samples where available. The 100 pg/mL threshold for clinical significance is indicated by a dashed line. FIG. 12D Correlation analysis of all amyloid CMP serum CLU concentrations with echocardiography left ventricular mass (LVM) measurements (Pearson r=−0.2248, one-tailed P<0.05, n=59). *P<0.05, **P<0.01, ***P<0.001.A.

FIG. 13 is a block diagram showing an example of a system for assessing a subject at risk of having cardiac amyloid deposits, and determining the custerin protein level in a biological sample (e.g., serum sample) from a subject and whether the subject has, or is at risk of having cardiac amyloid deposits. The system 10 comprises a determination module 20 which is configured to receive a biological sample, and measures the levels of clusterin protein in a biological sample from the subject, and outputs the information of the level of the clusterin protein, which is received and stored by the storage device 30 (where the storage device can also store reference clusterin level data), and a comparison module which receives the input from the storage device 30 and compares the clusterin level information from the biological sample with at least one reference clusterin level data, and provides information to an output module 50, wherein the output module can transmit the information to an end user, e.g., via a network e.g., world wide web, or produce a report, e.g., printed report, where the output information 60 displayed to the user can include one or more of the following information: (a) absolute clusterin levels (e.g., mg/ml clusterin levels in the biological sample, and optionally reference clusterin level), (b) clusterin level as a % of the reference clusterin level (e.g., 70% of reference clusterin level), (c) decrease (or increase) of clusterin level as compared to reference clusterin level, where the decrease (or increase) can be a % decrease (e.g., 30% decrease (or −30% or 2% increase (or +2%)) or absolute number decrease (e.g., 0.3 mg/mldecrease), or a fold decrease, (e.g., 1.5-fold or 2-fold decrease), (d) a positive signal to indicate cardiac amyloid deposits (e.g., a * or other highlight or + mark) or (e) a negative signal to indicate absence of cardiac amyloid deposits (e.g., absence of mark or other highlight (distinct from the positive indicator) or − mark).

FIG. 14 is a block diagram showing exemplary instructions on a computer readable medium for assessing clusterin protein level in a biological sample and whether a subject has, or is at risk of developing cardiac amyloid deposits (i.e. a positive or negative cardiac amyloid deposit test result). The computer readable medium can comprise instructions 70 which can be read by a computer to perform a method for determining if a subject has, or is at risk of developing cardiac amyloid deposits. The comparison module 40 which receives the input from the storage device 30 compares the clusterin level information from the biological sample (measured by the determination module) with at least one reference clusterin level data, and the comparison module determines if the measured clusterin level is below the reference clusterin level data, and optionally if the measured clusterin level is significantly below the reference clusterin level data (e.g., below a predetermined threshold reference clusterin level, e.g., where the threshold reference clusterin level can be 0.6 mg/ml (for serum levels)). The comparison module can provide data to the output module 50 that the subject from whom the biological sample was obtained has a positive test result (e.g., likely to have cardiac amyloid deposits) or a negative test result (e.g., likely not to have cardiac amyloid deposits). The comparison module outputs data content to the output module 50, which displays information 60 to the user, e.g., end user.

### DETAILED DESCRIPTION OF THE INVENTION

Embodiments of the present invention are based on the discovery that levels of clusterin (CLU) protein in a biological sample from a subject is useful for characterizing and identifying a subject at risk of developing, or having amyloidotic cardiomyopathy (CMP).

One aspect of the present invention is related to methods, systems and kits for detecting amyloidotic cardiomyopathy in a subject, e.g., a human subject. Provided herein are methods, systems and kits for determining if a subject has amyloidotic cardiomyopathy (CMP) or cardiac amyloid deposits, the method comprising measuring the level of clusterin protein in a biological sample from the subject, e.g., a blood or serum sample, where a low level of clusterin protein by a statistically significant level as compared to a reference level of clusterin level is indicative of the subject having cardiac amyloid deposit.

In some embodiments, the methods and systems as disclosed herein comprise an additional step of treating a subject identified as having cardiac amyloid deposits, and/or amyloidotic cardiomyopathy (CMP) by the methods and systems as disclosed herein, where the treating can comprise administering to the subject a pharmaceutical composition for treatment of disorders characterized by amyloidosis in the heart and cardiac-related amyloidosis.

Accordingly, one aspect of the present invention relates to a method for assessing a subject at risk of having cardiac amyloid deposits, the method comprising measuring the level of clusterin protein in a biological sample obtained from the subject, wherein a decrease in the level of clusterin protein in the biological sample a statistically significant amount as compared to a reference level of clusterin protein is indicative of the subject being at risk of having cardiac amyloid deposits. In some embodiments, if the level of the clusterin protein in the biological sample is decrease by more than 40%, or more than 50% or more than 60% as compared to a reference level of clusterin protein, it is indicative of the subject being at risk of having cardiac amyloid deposits.

In some embodiments, a level of the clusterin protein of less than about 0.5 mg/mL, or less than about 0.4 mg/mL, or less than about 0.3 mg/mL, or less than about 0.1 mg/mL, or between about 0.4 mg/mL-0.01 mg/mL in a biological sample, e.g., serum sample, is indicative of the subject being at risk of having cardiac amyloid deposits.

In some embodiments, the subject is a mammalian subject, for example a human subject. In some embodiments, the biological sample can be any peripheral fluid sample obtained from the subject, and can be a blood sample, plasma sample or a serum sample.

Typically, in some embodiments, the level of level of clusterin protein is measured by a protein binding molecule, for example but not limited to, an antibody, or an antibody fragment or a ligand binding partner of clusterin, or functional fragments thereof. In some embodiments, the level of clusterin protein is measured by immunoassay, for example an ELISA, however other methods to measure clusterin protein levels are encompassed in the methods and systems as disclosed herein.

In some embodiments, a subject identified as having amyloidotic cardiomyopathy using the kits, systems and methods as disclosed herein can be optionally administered a pharmaceutical composition for treatment amyloidotic cardiomyopathy. In some embodiments, such a treatment is a composition comprising clusterin, or a clusterin agent (e.g. an agonist of clusterin activity or a biologically active fragment or derivative thereof).

Another aspect of the present invention provides pharmaceutical compositions and methods for prophylactic and therapeutic treatment of disorders characterized by transthyretin (TTR) amyloidosis. More specifically, the present invention provides methods and compositions using clusterin, or a clusterin agent (e.g. an agonist of clusterin activity or a biologically active fragment or derivative thereof), for the treatment and/or prevention of a disease or disorder characterized by transthyretin (TTR) amyloidosis, e.g. senile systemic amyloidosis (SSA) or familial amyloidodic polyneuropathy.

Another aspect of the present invention relates to methods and kits for the diagnosis and/or prognosis of a cardiac-related amyloidosis by measuring clusterin (CLU) in a biological sample from a subject, such as a serum sample.

As previously reported in Lee et al., clusterin protein can decrease TTR amyloidosis and decrease the formation of TTR fibrils in neuronal cells and can be used to decrease TTR fibril formation for the treatment and/or prevention of TTR amyloidosis diseases such as SSA and FAP. Lee et al., also reported that clusterin increases the stability of tetrameric form of TTR, thus decreasing the amount of monomeric TTR available for TTR amyloid formation, and reported that clusterin functions to stabilize both wild-type TTR (e.g. in the case of SSA) and mutant TTR or TTR variants (e.g. in the case of FAP) in acid-conditions, e.g. at pH of below about pH 7.0, or at a pH of below about pH 6.0, or at a pH of below about pH 5.0, or at a pH of below about pH 4.0. However unlike the present invention, Lee et al., did not report the role of clusterin or levels of clusterin in subjects with amyloid associated diseases, or in amyloidotic cardiomyopathy.

Accordingly, one aspect of the present invention is based upon the discovery that clusterin is present at low levels in the serum of subjects with amyloidoitic cardiomyopathy and familial amyloid cardiomyopathy (FAC). In particular, clusterin was discovered to be associated with cardiac amyloid deposits in subjects and that serum levels of clusterin are significantly lower in the serum from subjects with amyloidotic cardiomyopathy, such as SSA subjects, ATTR subjects, AL amyloidosis subjects with amyloidotic cardiomyopathy as compared to age-matched subjects without cardiac amyloid disease.

Accordingly, one aspect of the present invention relates to identification of subjects with amyloidotic cardiomyopathy using the methods, systems and kits as disclosed herein and to provide a suitable prophylactic or therapeutic method for diseases or disorders which are connected or caused by TTR variant proteins, wild-type TTR proteins, immunoglobulin light chain proteins, fragments or aggregated proteins thereof, or at least, to provide temporal amelioration or slowing down of progression of the symptoms being connected with the protein aggregation of TTR or immunoglobulin light chain amyloid diseases and cardiac amyloid disorders, including but not limited to, senile systemic amyloidosis (SSA) and/or familial amyloid cardiomyopathy (FAC) and/or familial amyloid neuropathy (FAP).

Therefore, one aspect of the present invention provides the use of a clusterin protein, or a compound with clusterin activity (e.g., a clusterin analogue, mutant, variant or clusterin fragment) or a compound promoting or enhancing clusterin activity (e.g. a clusterin agonist) for the preparation of a medicament for treatment or prevention of diseases and disorders of cardiac amyloidosis, including TTR-mediated forms, e.g. senile systemic amyloidosis (SSA) and/or familial amyloid cardiomyopathy (FAC) and/or familial amyloid neuropathy (FAP). In some embodiments, a disease or disorders of TTR amyloidosis is familial amyloid neuropathy, familial amyloid cardiomyopathy (FAC) and familial central nervous system amyloidosis. Another aspect of the present invention provides the use of a clusterin protein, or a compound with clusterin activity (e.g., a clusterin analogue, mutant, variant or clusterin fragment) or a compound promoting or enhancing clusterin activity (e.g. a clusterin agonist) for the preparation of a medicament for treatment or prevention of diseases and disorders of immunoglobulin light chain amyloidosis, for example, AL or primary or light chain amyloidosis.

Clusterin has the ability to bind misfolded proteins of varying conformations, including misfolded TTR or TTR variants (e.g. V50M and L55P variants). Although the physiological function of clusterin as a binding protein to abnormal proteins preferably takes place in the extracellular space (and extracellular space is the preferred locus of clusterin action according to the present invention), also intracellular action of clusterin is enabled by the present invention as well as the direction or the targeting to specific cell compartments of cells, e.g. by providing suitable modifications in the signal sequence, such as deletion or truncation of the signal sequence.

### DEFINITION OF TERMS

Unless otherwise indicated, all terms used herein have the same meaning as they would to one skilled in the art of the present invention. Practitioners are particularly directed to Sambrook, et al. (1989) Molecular Cloning: A Laboratory Manual (Second Edition), Cold Spring Harbor Press, Plainview, N.Y. and Ausubel, F. M., et al. (1998) Current Protocols in Molecular Biology, John Wiley Sons, New York, N.Y., for definitions, terms of art and standard methods known in the art of biochemistry and molecular biology. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N.Y. (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. Unless otherwise indicated, nucleic acids are written left to right in 5′ to 3′ orientation; amino acid sequences are written left to right in amino to carboxy orientation, respectively. The headings provided herein are not limitations of the various aspects or embodiments of the invention which can be had by reference to the specification as a whole. Accordingly, the terms defined immediately below are more fully defined by reference to the specification as a whole. It is understood that this invention is not limited to the particular methodology, protocols, and reagents described, as these may be varied to produce the same result.

The term “amyloidosis” as used herein refers to a condition in which abnormal protein deposits in various tissues. These protein deposits damage the tissues and interfere with the function of the involved organ. The abnormal protein deposits are called amyloid, hence the name of this group of diseases. Deposits may be localized to one organ or distributed in a systemic manner to multiple tissues throughout the body. Typically, organs most often affected include, but are not limited to the heart, kidneys, nervous system, gastrointestinal tract, and soft tissues. There are several major types of systemic amyloid diseases including immunoglobulin light chain (AL) amyloidosis, the most common, and other familial forms which feature mutations in the genes of abundant serum proteins such as TTR, fibrinogen, and apolipoproteins. AL amyloidosis is a plasma cell malignancy in which clonally expanded bone marrow cells overexpress a monoclonal light chain that is prone to amyloid fibril formation. The familial forms are autosomal dominant diseases; the most frequently occurring is TTR-associated (ATTR) amyloidosis. Currently, there are more than 100 TTR gene mutations linked to ATTR including V30M, V122I (prevalent in the African-American population), and L55P. These TTR variants become highly destabilized and self-associate into aggregates which precipitate as amyloid deposits. Senile systemic amyloidosis (SSA) is a form a TTR-associated amyloidosis whereby the amyloidogenic protein is the wild-type, non-mutant form of TTR manifesting as cardiac amyloidosis.

The term “amyloid disease” or “amyloidosis” refers to any of a number of disorders which have as a symptom or as part of its pathology the accumulation or formation of plaques or amyloid plaques or accumulation of fibril. An “amyloid plaque” is an extracellular deposit composed mainly of proteinaceous fibrils. Generally, the fibrils are composed of a dominant protein or peptide; however, the plaque may also include additional components that are peptide or non-peptide molecules, as described herein.

The term “TTR amyloidosis” as used herein refers to any disease or disorders which has accumulation of transthyretin (TTR) amyloid fibril deposits. Typically, TTR amyloid deposits are the result of aggregation of mutant variants of TTR protein causing familial amyloidosis cardiomyopathy (FAC) or familial amyloidosis polyneuropathy (FAP), whereas TTR amyloid deposits from accumulation of wild-type TTR protein causes to senile systemic amyloidosis (SSA). TTR amyloidosis can occur in the third or fourth decade, but age of onset and clinical features can vary, and depend on the particular TTR mutation and/or ethnicity. TTR amyloidosis is a disease that has a high prevalence in certain ethnic groups such as African Americans. A common presentation in patients with ATTR amyloidosis is neuropathy which typically manifests as autonomic or sensory motor impairment. Autonomic manifestations include orthostatic hypotension, gastrointestinal abnormalities, carpal tunnel syndrome, inability to sweat, nephropathy, and urinary retention or incontinence. Sensory neuropathy typically begins in the lower extremities with paresthesia (pins and needles sensation) and motor neuropathy eventually ensues within a few years. ATTR amyloidosis can result in central nervous system manifestations such as hydrocephalus, dementia, psychosis, seizures, visual impairment and ataxia. Cardiac involvement is associated with more than half of the amyloidogenic TTR mutants described to date (manifesting as FAC) and it is the predominating type of involvement in patients with senile systemic amyloidosis. TTR amyloid deposits in the myocardium most often cause restrictive cardiomyopathy. The enlarged appearance of the interventricular septum and left ventricular muscle reflects the deposition of TTR in these structures rather than true hypertrophy. The symmetrical pattern of enlargement and histological appearance help differentiate this condition from other forms of hypertrophic cardiomyopathy. Additional cardiac symptoms can include arrhythmias, congestive heart failure and sudden death. TTR amyloidosis is also referred to under the synonyms; Familial Amyloid Cardiomyopathy (FAC), Familial Amyloid Polyneuropathy Type I (Portuguese-Swedish-Japanese Type), Familial Amyloid Polyneuropathy Type II (Indiana/Swiss or Maryland/German Type), Leptomeningeal Amyloidosis, Familial Oculoleptomeningeal Amyloidosis (FOLMA), Dysprealbunemic Euthyroid Hyperthyroxinemia, Dysprealbunemic Hyperthyroxinemia, Dystransthyretinemic Hyperthyroxinemia, Amyloid Polyneuropathy, Senile Systemic Amyloidosis (SSA).

The term “AL amyloidosis” or “immunoglobulin light chain amyloidosis” or “primary amyloidosis” as used herein refers to the disease or disorder from AL amyloid deposits, or the formation of amyloid deposits comprising monoclonal immunoglobulin light chain or fragments thereof.

The term “amyloid cardiomyopathy” or “amyloidotic cardiomyopathy” or “amyloid CMP” are used interchangeably herein and refer to a myocardial disease caused by infiltration of amyloid. Amyloidotic cardiomyopathy can also be referred to in the art as “cardiac amyloidosis”, “amyloidosis cardiac”, “primary cardiac amyloidosis—AL type”, “secondary cardiac amyloidosis—AA type”, “stiff heart syndrome” or “senile amyloidosis” or “senile cardiac amyloidosis” and is a disorder caused by deposits of an abnormal protein (amyloid) in the heart tissue, which make it difficult for the heart to function properly.

An “amyloid component” is any molecular entity that is present in an amyloid plaque including antigenic portions of such molecules. Amyloid components include but are not limited to proteins, peptides, proteoglycans, and carbohydrates. A “specific amyloid component” refers to a molecular entity that is found primarily or exclusively in the amyloid plaque of interest.

A “fibril peptide” or “fibril protein” or “amyloid fibrils” refers to an aggregated form of a protein or peptide that forms fibrils present in plaques or deposits. The fibrils may comprise monomeric, dimeric, or multimeric subunits, as well as fragments of the protein, e.g. TTR amyloid fibrils can comprise monomeric and/or dimeric and/or tetrameric and/or fragmented forms of TTR variant proteins.

The term ‘disorder’ or ‘disease’ used interchangeably herein, refers to any alteration in the state of the body or of some of its organs, interrupting or disturbing the performance of the functions and/or causing symptoms such as discomfort, dysfunction, distress, or even death to the person afflicted or those in contact with the person. A disease or disorder can also relate to distemper, ailing, ailment, malady, disorder, sickness, illness, complaint, indisposition, affection.

As used herein, the terms “homologous” or “homologues” are used interchangeably, and when used to describe a polynucleotide or polypeptide, indicates that two polynucleotides or polypeptides, or designated sequences thereof, when optimally aligned and compared, for example using BLAST, version 2.2.14 with default parameters for an alignment (see herein) are identical, with appropriate nucleotide insertions or deletions or amino-acid insertions or deletions, in at least 70% of the nucleotides or amino acid residues, usually from about 75% to 99%, and more preferably at least about 98 to 99% of the nucleotides or amino acid residues. The term “homolog” or “homologous” as used herein also refers to homology with respect to structure and/or function. With respect to sequence homology, sequences are homologs if they are at least 50%, at least 60%, at least 70%, at least 80%, at least 90%, at least 95% identical, at least 97% identical, or at least 99% identical. Determination of homologs of the genes or peptides of the present invention can be easily ascertained by the skilled artisan. Homologous sequences can be the same functional gene in different species.

The term “analog” as used herein of a molecule refers to a molecule similar in function to either the entire molecule of a fragment thereof. The term “analogue” is indented to include allelic, species and variants. Analogs typically differ from naturally occurring peptides at one or a few positions, often by virtue of conservative substitutions. Analogs typically exhibit at least 80 or 90% sequence identity with the natural peptides or the peptide sequence they are an analogue of. In some embodiments, analogs also include unnatural amino acids or modifications of N or C terminal amino acids. Examples of unnatural amino acids are acedisubstituted amino acids, N-alkyl amino acids, lactic acid, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, δ-N-methylarginine. Fragments and analogs can be screened for prophylactic or therapeutic efficacy in transgenic animal models as described below.

The term “substantial identity” as used herein refers to two peptide sequences, when optimally aligned, such as by the programs GAP or BESTFIT using default gap weights, share at least about 65%, at least about . . . 70%, at least about . . . 80%, at least about . . . 90% sequence identity, at least about . . . 95% sequence identity or more (e.g., 99% sequence identity or higher). In some embodiments, residue positions which are not identical differ by conservative amino acid substitutions.

The term “antibody” is used to include intact antibodies and binding fragments thereof. Typically, fragments compete with the intact antibody from which they were derived for specific binding to an antigen. Optionally, antibodies or binding fragments thereof, can be chemically conjugated to, or expressed as, fusion proteins with other proteins.

The term “epitope” or “antigenic determinant” refers to a site on an antigen to which B and/or T cells respond. The term can also refer to the part of an antigen that binds to the antigen-binding region of an antibody. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., J Inf. Dis. 170, 1110-19 (1994)), by antigen-dependent killing (cytotoxic T lymphocyte assay, Tigges et al., J. Immunol. 156, 3901-3910) or by cytokine secretion.

The term “immunological” or “immune” as used herein with respect to an immunological or immune response, refers to the development of a humoral (antibody mediated) and/or a cellular (mediated by antigen-specific T cells or their secretion products) response directed against an amyloid peptide in a recipient subject. Such a response can be an active response induced by administration of an immunogen to a subject or a passive response induced by administration of antibody or primed T-cells that are directed towards the immunogen. A cellular immune response is elicited by the presentation of polypeptide epitopes in association with Class I or Class II MHC molecules to activate antigen-specific CD4⁺ T helper cells and/or CD8⁺ cytotoxic T cells. Such a response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays (see Burke, supra; Tigges, supra). The relative contributions of humoral and cellular responses to the protective or therapeutic effect of an immunogen can be distinguished by separately isolating IgG and T-cells from an immunized syngeneic animal and measuring protective or therapeutic effect in a second subject, or a cytotoxic T-cell response (CTL). Thus, a peptide or protein which is “immunogenic”, “agent” or an “immunogen” as used herein induces an immunological response against itself on administration to a subject.

An “agent” is a chemical molecule of synthetic or biological origin. In the context of the present invention, an agent is generally a molecule that can be used in a pharmaceutical composition.

The terms “polynucleotide” and “nucleic acid,” as used interchangeably herein refer to a polymeric molecule having a backbone that supports bases capable of hydrogen bonding to typical polynucleotides, where the polymer backbone presents the bases in a manner to permit such hydrogen bonding in a sequence specific fashion between the polymeric molecule and a typical polynucleotide (e.g., single-stranded DNA). Such bases are typically inosine, adenosine, guanosine, cytosine, uracil and thymidine. Polymeric molecules include double and single stranded RNA and DNA, and backbone modifications thereof, for example, methylphosphonate linkages.

The term “polypeptide” or “peptide” or “protein” as used herein refer to a polymer of amino acid residues linked by peptide bonds, and are not limited to a minimum length. The term “protein” may refer to a complex of two or more polypeptides, e.g., dimers, multimers, oligopeptides, and the like, are also composed of linearly arranged amino acids linked by peptide bonds, and whether produced biologically, recombinantly, or synthetically and whether composed of naturally occurring or non-naturally occurring amino acids, are included within this definition. Both full-length proteins and fragments thereof are encompassed by the definition. The terms also include co-translational (e.g., signal peptide cleavage) and post-translational modifications of the polypeptide, such as, for example, disulfide-bond formation, glycosylation, acetylation, phosphorylation, proteolytic cleavage (e.g., cleavage by furins or metalloproteases), and the like. Furthermore, for purposes of the present invention, a “polypeptide” refers to a protein that includes modifications, such as deletions, additions, and substitutions (generally conservative in nature as would be known to a person in the art), to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods. Polypeptides or proteins are composed of linearly arranged amino acids linked by peptide bonds, but in contrast to peptides, has a well-defined conformation. Generally, peptides contain at least two amino acid residues and are less than about 100 amino acids, more preferably less than about 50 amino acids in length. Proteins, as opposed to peptides, generally consist of chains of 100 or more amino acids. The terms apply to amino acid polymers in which one or more amino acid residue is an artificial chemical analog of a corresponding naturally occurring amino acid, as well as to naturally occurring amino acid polymers. For the purposes of the present invention, the term “peptide” or “protein” as used herein typically refers to a sequence of amino acids of made up of a single chain of D- or L-amino acids or a mixture of D- and L-amino acids joined by peptide bonds. As used herein, the term “protein fragment” may also be read to mean a peptide.

The incorporation of non-natural amino acids, including synthetic non-native amino acids, substituted amino acids, or one or more D-amino acids into the peptides (or other components of the composition, with exception for protease recognition sequences) is desirable in certain situations. D-amino acid-containing peptides exhibit increased stability in vitro or in vivo compared to L-amino acid-containing forms. Thus, the construction of peptides incorporating D-amino acids can be particularly useful when greater in vivo or intracellular stability is desired or required. More specifically, D-peptides are resistant to endogenous peptidases and proteases, thereby providing better oral trans-epithelial and transdermal delivery of linked drugs and conjugates, improved bioavailability of membrane-permanent complexes (see below for further discussion), and prolonged intravascular and interstitial lifetimes when such properties are desirable. The use of D-isomer peptides can also enhance transdermal and oral trans-epithelial delivery of linked drugs and other cargo molecules. Additionally, D-peptides cannot be processed efficiently for major histocompatibility complex class II-restricted presentation to T helper cells, and are therefore less likely to induce humoral immune responses in the whole organism. Peptide conjugates can therefore be constructed using, for example, D-isomer forms of cell penetrating peptide sequences, L-isomer forms of cleavage sites, and D-isomer forms of therapeutic peptides. In some embodiments, peptides can be comprised of D- or L-amino acid residues, as use of naturally occurring L-amino acid residues has the advantage that any break-down products should be relatively non-toxic to the cell or organism.

A “glycoprotein” as use herein is protein to which at least one carbohydrate chain (oligopolysaccharide) is covalently attached. A “proteoglycan” as used herein is a glycoprotein where at least one of the carbohydrate chains is a glycosaminoglycan, which is a long linear polymer of repeating disaccharides in which one member of the pair usually is a sugar acid (uronic acid) and the other is an amino sugar.

The term “subject” includes human and other mammalian subjects who can be assessed for cardiac amyloid deposits, and subjects who are amenable to receive treatment, e.g., therapeutic and/or preventative (e.g., prophylactic) treatment. The term “subject” and “individual” are used interchangeably herein, and refer to an animal, for example a human, to whom treatment, including prophylactic treatment, with the cells according to the present invention, is provided. For treatment of conditions or disease states which are specific for a specific animal such as a human patient, the term patient refers to that specific animal. The “non-human animals” of the invention include mammals such as rats, mice, rabbits, sheep, cats, dogs, cows, pigs, and non-human primates.

The term “effective amount” or “therapeutically effective amount” as used herein refers to the amount of therapeutic agent (e.g. clusterin protein or clusterin agents) of pharmaceutical composition to alleviate at least some of the symptoms of the disease or disorder of TTR amyloidosis. Stated another way, “therapeutically effective amount” of clusterin protein or clusterin agent is the amount of clusterin protein or clusterin agent which exerts a beneficial effect on the TTR amyloidic disease. The dosage administered, as single or multiple doses, to an individual will vary depending upon a variety of factors, including clusterin pharmacokinetic properties, the route of administration, patient conditions and characteristics (sex, age, body weight, health, size), extent of symptoms, concurrent treatments, frequency of treatment and the effect desired.

The term “treating” as used herein is intended to encompass curing as well as ameliorating at least one symptom of the condition or disease. For example, in the case of TTR amyloidosis, treatment includes a reduction in the amount or the production of TTR amyloid fibrils. Evidence of treatment may be clinical or sub-clinical. The term “treatment” refers generally to afflicting a subject, tissue or cell to obtain a desired pharmacologic and/or physiologic effect. Stated another way, the terms “treat,” “treating,” and “treatment” refer to the alleviation or measurable lessening of one or more symptoms or measurable markers of a disease or disorder; while not intending to be limited to such, disease or disorders of particular interest include ischemic or ischemia/reperfusion injury and diabetes. Measurable lessening includes any statistically significant decline in a measurable marker or symptom. In some embodiments, a measurable marker is levels of serum clusterin levels, e.g., serum clusterin protein levels, and a decline a symptom is an increase in serum clusterin protein levels to within normal serum clusterin levels, e.g., close to a reference clusterin levels, for example, about 0.6 mg/ml clusterin protein level in the serum.

As used herein, the terms “prevent,” “preventing” and “prevention” refer to the avoidance or delay in manifestation of one or more symptoms or measurable markers of a disease or disorder. A delay in the manifestation of a symptom or marker is a delay relative to the time at which such symptom or marker manifests in a control or untreated subject with a similar likelihood or susceptibility of developing the disease or disorder. For example, in some embodiments, a preventative treatment is a treatment which prevents the formation of cardiac amyloid deposits, and/or prevents a decrease in serum clusterin protein levels in a subject with an amyloid-associated disease, for example, maintains the serum clusterin protein levels at a normal range, for example, a reference serum clusterin level of about 0.6-0.7 mg/ml. Thus, in some embodiments, a treatment may be prophylactic in terms of completely or partially preventing a disease or sign or symptom thereof or preventing cardiac amyloid deposits and/or amyloidotic cardiomyopathy, and/or may be therapeutic in terms of a partial or complete cure of a disease. The terms “prevent” “preventing” and “prevention” include not only the complete avoidance or prevention of symptoms or markers, but also a reduced severity or degree of any one of those symptoms or markers, relative to those symptoms or markers arising in a control or non-treated individual with a similar likelihood or susceptibility of developing the disease or disorder, or relative to symptoms or markers likely to arise based on historical or statistical measures of populations affected by the disease or disorder. By “reduced severity” is meant at least a 10% reduction in the severity or degree of a symptom or measurable disease marker, relative to a control or reference, e.g., at least 15%, 20%, 30%, 40%, 50%, 60%, 70%, 80%, 90%, 95%, 99% or even 100% (i.e., no symptoms or measurable markers).

The term “tissue” is intended to include intact cells, blood, blood preparations such as plasma and serum, bones, joints, muscles, smooth muscles, and organs.

The term “linker peptide” includes reference to a peptide within an antibody binding fragment (e.g., Fv fragment) which serves to indirectly bond the variable heavy chain to the variable light chain.

The term “naked polynucleotide” refers to a polynucleotide not complexed with colloidal materials. Naked polynucleotides are sometimes cloned in a plasmid vector.

The term “encode” as it is applied to polynucleotides refers to a polynucleotide which is said to “encode” a polypeptide or protein if, in its native state or when manipulated by methods well known to those skilled in the art, it can be transcribed to produce the RNA which can be translated into an amino acid sequence to generate the polypeptide and/or a fragment thereof. The antisense strand is the complement of such a nucleic acid, and the encoding sequence can be deduced therefrom.

The term “vector” refers to a nucleic acid molecule capable of transporting another nucleic acid to which it has been linked. Preferred vectors are those capable of autonomous replication and/or expression of nucleic acids to which they are linked Vectors capable of directing the expression of genes to which they are operatively linked are referred to herein as “expression vectors”.

The term “viral vectors” refers to the use as viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g. EPV and EBV vectors.

The term “pharmaceutical composition” refers to a chemical or biological composition suitable for administration to a mammalian subject. Such compositions may be specifically formulated for administration via one or more of a number of routes, including but not limited to, oral, parenteral, intravenous, intraarterial, subcutaneous, intranasal, sublingual, intraspinal, intracerebroventricular, and the like.

The term “pharmaceutically acceptable” is meant to encompass any carrier, which does not interfere with effectiveness of the biological activity of the active ingredient and that is not toxic to the host to which it is administered. For example, for parenteral administration, the active protein (s) may be formulated in a unit dosage form for injection in vehicles such as saline, dextrose solution, serum albumin and Ringer's solution.

The term “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically,” “peripheral administration” and “administered peripherally” as used herein mean the administration of a compound, drug or other material other than directly into the central nervous system, such that it enters the animal's system and, thus, is subject to metabolism and other like processes, for example, subcutaneous administration.

The terms “pharmaceutically acceptable carrier” “pharmaceutical excipient” or a “pharmaceutically acceptable excipient” are used interchangeably herein and refer to a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation. A carrier generally does not provide any pharmacological activity to the formulation, though it may provide chemical and/or biological stability, release characteristics, and the like. Exemplary formulations can be found, for example, in Remington's Pharmaceutical Sciences, 19^{th} Ed., Grennaro, A., Ed., 1999

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip.

The term “software” can refer to prescribed rules to operate a computer. Examples of software include: software; code segments; instructions; computer programs; and programmed logic.

The term a “computer system” may refer to a system having a computer, where the computer comprises a computer-readable medium embodying software to operate the computer.

The term “proteomics” may refer to the study of the expression, structure, and function of proteins within cells, including the way they work and interact with each other, providing different information than genomic analysis of gene expression.

The terms “lower”, “reduced”, “reduction” or “decrease” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. When “decrease” or “inhibition” is used in the context of the level of expression or activity of a gene, it refers to a reduction in protein or nucleic acid level or activity in a cell, a cell extract, or a cell supernatant. For example, such a decrease may be due to reduced RNA stability, transcription, or translation, increased protein degradation, or RNA interference. Preferably, this decrease is at least about 5%, at least about 10%, at least about 25%, at least about 50%, at least about 75%, at least about 80%, or even at least about 90% of the level of expression or activity under control conditions.

The terms “increased”, “increase” or “enhance” or “higher” are all used herein to generally mean an increase by a statically significant amount; for the avoidance of any doubt, the terms “increased”, “increase” or “enhance” or “higher” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. When “increase” is used in the context of the expression or activity of a gene or protein, it refers to a positive change in protein or nucleic acid level or activity in a cell, a cell extract, or a cell supernatant. For example, such an increase may be due to increased RNA stability, transcription, or translation, or decreased protein degradation. Preferably, this increase is at least 5%, at least about 10%, at least about 25%, at least about 50%, at least about 75%, at least about 80%, at least about 100%, at least about 200%, or even about 500% or more over the level of expression or activity under control conditions.

The terms “significantly different than,” “statistically significant,” “significantly lower (or higher) than,” and similar phrases refer to comparisons between data or other measurements, wherein the differences between two compared individuals or groups are evidently or reasonably different to the trained observer, or statistically significant (if the phrase includes the term “statistically” or if there is some indication of statistical test, such as a p-value, or if the data, when analyzed, produce a statistical difference by standard statistical tests known in the art). To avoid any doubt, the term “statistically significant” or “significantly” refers to statistical significance and generally means a two standard deviation (2SD) below normal, or lower, than the normal concentration of a marker, e.g., normal clusterin levels.

The term “substantially” as used herein means a proportion of at least about 60%, or preferably at least about 70% or at least about 80%, or at least about 90%, at least about 95%, at least about 97% or at least about 99% or more, or any integer between 70% and 100%.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and the include plural references unless the context clearly dictates otherwise. Thus for example, references to “the method” includes one or more methods, and/or steps of the type described herein and/or which will become apparent to those persons skilled in the art upon reading this disclosure and so forth.

The articles “a” and an are used herein to refer to one or to more than one (i.e., to at least one) of the grammatical object of the article. By way of example, “an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

The terms “comprising” means “including principally, but not necessary solely”. Furthermore, variation of the word “comprising”, such as “comprise” and “comprises”, have correspondingly varied meanings. The term “consisting essentially” means “including principally, but not necessary solely at least one”, and as such, is intended to mean a “selection of one or more, and in any combination.” Compositions or methods “comprising” one or more recited elements may include other elements not specifically recited. For example, a composition that comprises a fibril component peptide encompasses both the isolated peptide and the peptide as a component of a larger polypeptide sequence. By way of further example, a composition that comprises elements A and B also encompasses a composition consisting of A, B and C.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Other features and advantages of the invention will be apparent from the following Detailed Description, the drawings, and the claims.

### Cardiac Amyloidosis

Cardiac amyloidosis is a disorder caused by deposits of an abnormal protein (amyloid) in the heart tissue, which make it hard for the heart to work properly, and is caused by amyloidosis, which refers to a family of diseases in which there is a buildup of clumps of proteins called amyloids in body tissues and organs. These proteins slowly replace normal tissue, leading to failure of the involved organ. There are many forms of amyloidosis, as discussed herein. However, cardiac amyloidosis frequently occurs during primary amyloidosis (e.g., AL type amyloidosis), which can often occur in people with multiple myeloma cancer.

Cardiac amyloidosis (e.g., “stiff heart syndrome”) occurs when amyloid deposits take the place of normal heart muscle. It is the most typical type of restrictive cardiomyopathy (RCM). Cardiac amyloidosis may affect the way electrical signals move through the heart (conduction system). This can lead to arrhythmias and conduction disturbances (heart block). Cardiac amyloidosis in the age-related form (SSA) is more common in men than in women. The disease is rare in people under age 40.

Amyloidosis refers to the extracellular tissue deposition of fibrils that are composed of low molecular weight subunits (5 to 25 kD) of a variety of serum proteins. These fibrils are organized in a beta-pleated sheet configuration and tissue that is infiltrated by amyloid deposits display characteristic histologic changes. Amyloid deposits can occur in a variety of organs, including the heart, kidney, liver, and autonomic nervous system, causing morbidity and mortality. The identity of the amyloidogenic protein, i.e. the protein that forms fibrils and deposits, varies with the type of amyloidosis.

In AL (primary) amyloidosis, a plasma cell dyscrasia, the amyloid protein is a monoclonal immunoglobulin light chain which is expressed by clonal plasma cells in the bone marrow. The amyloid deposits in AL amyloidosis are composed of monomeric light chain and fragmented portions of the protein. In secondary or AA amyloidosis, the amyloid protein in the deposits is derived from serum amyloid A (SAA), an acute phase reactant which circulates mainly in association with HDL. Senile systemic or senile cardiac amyloidosis is caused by transthyretin (formerly known as prealbumin) protein. Moreover, senile systemic amyloidosis (SSA) is caused by destabilization of a TTR protein, and is thought to be greatly unrecognized as an advanced-age disorder, as the incidence of SSA may increase in prevalence as the average age of the population increases. Cardiac involvement is common in primary amyloidosis, senile systemic amyloidosis (SSA), and inherited forms of amyloidosis, but clinically significant cardiac involvement is uncommon in secondary amyloidosis. Cardiac involvement may be the cause of heart failure in the senile form.

Cardiac amyloidosis can occur, albeit rarely, in secondary or reactive amyloidosis (e.g., the AA type) and familial apolipoprotein AI amyloidosis. Fragments of serum amyloid A protein, an acute phase reactant, are responsible for AA (secondary) amyloidosis, which is associated with a variety of chronic inflammatory disorders.

Mutations in the gene for transthyretin (previously called prealbumin) are often associated with heart disease and, with some mutations, organ involvement is primarily cardiac-related. Wild-type transthyretin is responsible for senile systemic amyloidosis, the predominant feature of which is an infiltrative cardiomyopathy.

A subject with cardiac amyloidosis may experience one or more of the following symptoms, including but not limited to, excessive urination at night, fatigue, reduced activity tolerance, palpitations (sensation of feeling heart beat), shortness of breath with activity, swelling of legs, ankles, or other part of the body (e.g., abdominal swelling), trouble breathing while lying down, however, some patients may have no symptoms. Other symptoms of a subject with cardiac amyloidosis include one or more of the following signals: abnormal sounds in the lung (lung crackles) or a heart murmur, blood pressure that is low or drops when you stand up, enlarged neck veins, swollen liver, dizziness when you change position, excessive weight (fluid) gain, excessive weight loss, fainting spells, and severe breathing difficulty.

Subjects diagnosed with cardiac amyloidosis may suffer from other complications and diseases, including but not limited to, atrial fibrillation or ventricular arrhythmias, congestive heart failure, fluid buildup in the abdomen (ascites), increased sensitivity to digoxin, low blood pressure and dizziness from excessive urination, sick sinus syndrome, symptomatic cardiac conduction system disease (arrhythmias related to abnormal conduction of impulses through the heart muscle).

Treatment of amyloidotic cardiomyopathy. A physician will recommend a change in life style and alteration in the subjects diet, for example, to reduce salt and fluid intake. A physician may proscribe water pills (diuretics) to help your body remove excess fluid. A physician may also administer any one of the following treatments, digoxin, calcium channel blockers, and beta blockers, chemotherapy, implantable cardioverter-defibrillator (AICD), pacemaker, if there are problems with heart signals, prednisone, an anti-inflammatory medicine, and in some cases, a heart transplant may be considered for some patients with very poor heart function, unless the subject has AL type amyloidosis, as this weakens many other organs. People with hereditary amyloidosis can undergo a liver transplant.

Cardiac amyloidosis can be difficult to diagnose because the signs can be related to a number of different conditions. Moreover, determination of the type of cardiac amyloidosis, i.e. identification of the amyloid fibril protein is critical as prognosis and management strategies vary. In TTR-associated cardiac amyloidosis, the disease can be a long-term chronic condition that progresses unabated with worsening manifestations of disrupted organ function. Current diagnosis of amyloidotic cardiomyopathy is complex and relies on expensive scans utilizing specialized imaging equipment in addition to a variety of invasive techniques using histopathological, immunological, and biochemical testing procedures; there is no single test that easily identifies a subject with cardiac amyloidosis. Current diagnosis of cardiac amyloidosis include, one or a combination of, a chest or abdomen CT scan, coronary angiography, echocardiogram, magnetic resonance imaging (MRI), nuclear heart scans (MUGA, RNV), or an ECG, which may show problems with the heart beat or heart signals (conduction disturbance). In some instances, a cardiac biopsy is used to confirm the diagnosis, as well as a biopsy of another area, such as the abdomen, kidney, or bone marrow, is often done to confirm the diagnosis, and the biopsy tissue is stained with congo red. Cardiac amyloidosis is a long-term (chronic) condition that slowly gets worse, and a subject identified with cardiac amyloidosis, on average, live less than 1 year.

The most common manifestation of cardiac amyloidosis is restrictive cardiomyopathy (RCM). Systolic dysfunction dominates the cardiac manifestation less frequently. Other abnormalities include orthostatic hypotension, arrhythmias, conduction abnormalities and low ECG voltage. Plain radiography shows pulmonary venous hypertension or oedema with little or no cardiomegaly in the restrictive form. Cardiomegaly may be present in the form with primarily systolic dysfunction. Echocardiography demonstrates thickened atrial walls, especially on the right side; thickened ventricular walls; small left ventricular volumes (LV); decreased ejection fraction; and diffuse hypokinesis. Echocardiography may reveal a distinctive “speckled” texture of the myocardium. Doppler echocardiography and blood pool LV scintigraphy show decreased left ventricular (LV) diastolic indices. Technetium 99m pyrophosphate scans may show intense myocardial uptake. MRI has become especially effective for demonstrating the thickening of the right atrial wall, interatrial septum and LV myocardium. A characteristic feature is enlarged atria in the presence of small ventricular cavities. Because of the diastolic dysfunction causing slow emptying of the atria, prominent intracavitary signal in the atria is evident on spin echo images. Cine MRI demonstrate diffuse hypokinesis of the LV and velocity-encoded cine MRI documents the depressed diastolic filling indices.

Accordingly, one aspect of the invention as disclosed herein relates to systems, methods and kits to diagnose a subject with cardiac amyloidosis, or the onset of cardiac amyloidosis so that the subject can be administered effective treatment to prevent, or preferably, reverse the cardiac amyloidosis. In particular, one aspect of the present invention provides a method for the diagnosis of a subject with cardiac amyloid deposits, the method comprising measuring the level of clusterin protein in a biological sample obtained from the subject, wherein if the level of clusterin protein in the biological sample from the subject is lower by a statistically significant amount relative to a reference level of clusterin protein, the subject likely is at risk of having cardiac amyloid deposits.

Accordingly, one aspects of the present invention relates to a method for assessing a subject at risk of having cardiac amyloid deposits, for example, for risk of amyloidotic cardiomyopathy (CMP), the method comprising measuring the level of clusterin protein in a biological sample obtained from the subject, wherein a decrease in the level of clusterin protein in the biological sample a statistically significant amount as compared to a reference level of clusterin protein is indicative of the subject being at risk of having cardiac amyloid deposits. In some embodiments, a decrease in the level of the clusterin protein in the biological sample by more than about 40%, or more than about 50%, or more than about 60%, as compared to a reference level of clusterin protein is indicative of the subject being at risk of having cardiac amyloid deposits.

### Clusterin and Clusterin Agents

Clusterin (which is also known by aliases as pADHC-9, SGP-2, TRPM-2, SP-40,40, CLI, ApoJ (apolipoprotein J), T64, GP III, GPS0, XIP8) is a multifunctional protein, being constitutively expressed in almost all mammalian tissues and found in most human fluids (plasma, milk, urine, cerebrospinal fluid and semen) (Jones et al., 2002). The high degree of conservation (˜70-85% in mammalian species), the absence of polymorphism and the wide tissue distribution suggests a fundamental biological function of clusterin (Trougakos et al., 2002). It has been described as a lipid-transporter and cell adhesion molecule, and reported to bind bacteria, to be involved in apoptosis (pro- as well as anti-apoptotic function) and in numerous other processes, including stress response (Jones et al., 2002; Trougakos et al., 2002).

For the purposes of this disclosure, the term “clusterin” refers to the apolipoprotein-J originally derived from ram rete testes (NCBI Acc. No. NM_{—}203339, NM_{—}001831), and to homologous proteins derived from other mammalian species, including human, whether denominated as clusterin or not. The sequences of numerous clusterin species are known and have been assigned NCBI accession numbers (NCBI Acc. No. NM_{—}013492, NM_{—}053021, NM_{—}012679).

Without wishing to be bound to theory, a non-glycosylated splicing variant of clusterin appears in the nucleus (nClu), and this inactive precursor nClu exists in the cytoplasm and translocates to the nucleus following ionizing radiation, where the mature nClu interacts with the DNA-binding protein Ku70 and induces apoptosis (Leskov et al., 2003; Yang et al., 2000). These results are supported by the fact, that clusterin contains at least two nuclear localization signals.

A cytoplasmic localization (cClu) has been described (Debure et al., 2003; Trougakos et al., 2002), and the chicken clusterin homologue is not secreted, i.e., it is retained intracellularly (Mahon et al., 1999). An ubiquitinated form of clusterin forms juxtanuclear aggregates in the cytoplasm, similar to aggresomes (Debure et al., 2003). Clusterin could eventually escape the secretory pathway, particularly in pathologic situations with damage of the endoplasmic reticulum or modification of the chemical composition of the protein, and accumulate in the cytoplasmic compartment. Moreover, intracellular clusterin could also result through an uptake of extracellular protein.

The predominant form of clusterin is a secreted disulfide-linked heterodimeric glycoprotein (sClu) of 75-80 kDa (Jones et al., 2002). The single-copy gene with the coding sequence is located on human chromosome 8. The primary translation product is a polypeptide of 449 amino acids, including a hydrophobic 22-mer signal peptide. Secreted clusterin is translated on membrane-bound ribosomes directly in the endoplasmic reticulum with cleavage of the signal peptide. In the endoplasmic reticulum, the mannose-rich polypeptide is glycosylated and forms five disulfide bridges. After transport to the Golgi apparatus, clusterin is proteolytically cleaved, generating α and β subunits. After final glycosylation steps, the sClu (secreted clusterin) is secreted into the extracellular space (Jones et al., 2002; Trougakos et al., 2002; Wilson et al., 2000). Once secreted, clusterin can be bound by the megalin/gp330 receptor and taken up by the cell (Debure et al., 2003; Kounnas et al., 1995; Mahon et al., 1999).

Clusterin is a highly conserved (˜70-85% in mammalian species), multifunctional glycoprotein (Jones et al., 2002; Trougakos et al., 2002). sClu has chaperone-like activity. Secreted clusterin forms oligomers in the extracellular space. Active heterodimers dissociate from the inactive oligomers by acidic pH. sClu is able to bind to exposed hydrophobic regions of misfolded proteins in an ATP-independent manner and to stabilize them in a folding-competent state by forming soluble “high molecular weight complexes.” Similar to soluble heat shock proteins (sHsps), clusterin is unable to refold proteins. Cluster-bound proteins have been described to be competent for refolding by heat shock protein 70 (Hsp70), but an extracellular folding-competent chaperone has not been identified, yet. As opposed to Hsps, the chaperone activity of clusterin is not enhanced by increasing temperatures, but is by acidic pH. Thus, clusterin is the first identified extracellular chaperone (Humphreys et al., 1999; Poon et al., 2000; Poon et al., 2002; Wilson et al., 2000).

### Proteomic Screening and Measuring of Clusterin Protein Levels

One aspect of the present invention provides a method for the diagnosis of a subject with cardiac amyloid deposits, the method comprising measuring the level of clusterin protein in a biological sample obtained from the subject, wherein if the level of clusterin protein in the biological sample from the subject is lower by a statistically significant amount relative to a reference level of clusterin protein, the subject likely is at risk of having cardiac amyloid deposits.

Accordingly, one aspect of the present invention relates to a method for assessing a subject at risk of having cardiac amyloid deposits, for example, at risk for amyloidotic cardiomyopathy (CMP), the method comprising measuring the level of clusterin protein in a biological sample obtained from the subject, wherein a decrease in the level of clusterin protein in the biological sample by a statistically significant amount as compared to a reference level of clusterin protein is indicative of the subject being at risk of having cardiac amyloid deposits. In some embodiments, a decrease in the level of the clusterin protein in the biological sample by more than about 40%, or more than about 50%, or more than about 60%, as compared to a reference level of clusterin protein is indicative of the subject being at risk of having cardiac amyloid deposits.

In some embodiments, the amount of clusterin protein measured in a biological sample is compared to a reference level, or a reference biological sample, such as biological sample obtained from an age-matched normal control (e.g. an age-matched subject not having an amyloid disease), or a healthy subject, e.g., a healthy individual. In some embodiments, a reference value of clusterin protein in a serum sample is about 0.6 mg/mL. Thus, if there is a statistically significant decrease in clusterin protein level in a serum sample from a subject that is below 0.6 mg/mL, for example at a level of less than about 0.4 mg/mL, or less than about 0.3 mg/mL, or less than about 0.2 mg/mL, then the subject there is an indication that the subject is at risk of having cardiac amyloid deposits. Thus, if a test subject has at least a 70% less, or at least about a 60% less, or at least a 50% less, or at least about a 40% less or at least about 30% less or at least about 20% less or less than 20% of the level of the reference level of clusterin, then the subject likely to be at risk of having, or has cardiac amyloid deposits.

Stated another way, if the measured level of clusterin protein in the biological sample from the subject is lower (e.g., decreased) by a statistically significant amount than the reference amount of clusterin protein, e.g. by a decrease of about 20%, or a decrease of about 30%, or a decrease of about 40%, or a decrease of about 50%, or a decrease of about 60%, or a decrease of more than about 60% from the reference level of clusterin protein, then it is indicative of the subject being at risk of having cardiac amyloid deposits.

In some embodiments, the methods, systems and kits as disclosed herein also are useful for monitoring a course of treatment being administered to a subject. For example, one can measure the level of clusterin protein in a biological sample in the subject at a first timepoint (e.g., t1) and compare with a reference level, and if the measured level is significantly lower than the reference level, the subject can be administered an appropriate therapeutic treatment for treatment of amyloidosis cardiomyopathy (CMP), e.g., for example, clusterin and/or clusterin agonists as disclosed in the methods herein, and then the level of clusterin protein can be measured at a second (e.g., t2) and subsequent timepoints (e.g., t3, t4, t5, t5 . . . etc), and compared to levels of clusterin protein at one or more time points (e.g., at t1 or any subsequent timepoint) or the reference clusterin level to determine if a therapeutic treatment or medical treatment or regimen for the treatment of amyloidotic cardiomyopathy is effective. In some embodiments, the methods, systems and kits as disclosed herein can be used to monitor a therapeutic treatment in symptomatic subject (e.g., a subject showing at least one symptom of amyloidotic cardiomyopathy) where an effective treatment can be an increase clusterin serum levels and/or a decrease cardiac amyloid deposits in the subject, or alternatively the methods, systems and kits as disclosed herein can be used to monitor the effect of prophylactic treatment in asymptomatic subject (e.g., to prevent the formation of cardiac amyloid deposits in a subject and/or prevent a decrease in clusterin levels in a subject), for example, where the subject has a predisposition to develop amyloidotic cardiomyopathy.

In some embodiments, a biological sample for use in the methods and systems as disclosed herein is a peripheral biological fluid sample, for example, any one of the samples selected from: blood, plasma, serum, urine, mucus or cerebral spinal fluid obtained from the subject. A biological sample can be taken from any biological sample, e.g. a valid body tissue, especially body fluid, of a (human) subject, but preferably blood, plasma or serum. Other usable body fluids include cerebrospinal fluid (CSF), urine and tears.

According to another embodiment of the invention, the method, systems and diagnosis can be carried out post mortem on a biological sample from a deceased subject. In some embodiments, such biological sample can be pre-treated to extract proteins therefrom, including those that would be present in the blood of the deceased, so as to ensure that the relevant clusterin proteins specified above will be present in a positive sample. For the purposes of this patent specification, such an extract is equivalent to a body fluid.

Biological fluid samples, particularly peripheral biological fluid samples may be tested without prior processing of the sample as allowed by some assay formats. Alternatively, many peripheral biological fluid samples will be processed prior to testing. Processing generally takes the form of elimination of cells (nucleated and non-nucleated), such as erythrocytes, leukocytes, and platelets in blood samples, and may also include the elimination of certain proteins, such as certain clotting cascade proteins from blood. In some examples, the peripheral biological fluid sample is collected in a container comprising EDTA.

Subjects may be a mammal, such as a human, or a non-human subject. In some embodiments, a biological sample is analyzed for the level of clusterin protein, e.g. a protein of amino acids of SEQ ID NO: 1, or a homologue thereof.

One can use any proteomic approach commonly known to persons of ordinary skill in the art of measuring the level of clusterin protein in a biological sample.

As described herein, the level of clusterin protein is measured in a biological sample from a subject. The level of clusterin protein may be measured using any available measurement technology that is capable of specifically determining the level of clusterin protein in a biological sample. The measurement may be either quantitative or qualitative, so long as the measurement is capable of indicating whether the level of clusterin protein in the biological fluid sample is above or below the reference value.

The measured level of clusterin protein may be a primary measurement of the level of clusterin protein measuring the quantity of clusterin protein itself, such as by detecting the number of clusterin protein molecules in the sample) or it may be a secondary measurement of clusterin (a measurement from which the quantity of the clusterin protein can be but not necessarily deduced, such as a measure of enzymatic activity or a measure of nucleic acid, such as mRNA, encoding the clusterin protein). Qualitative data may also be derived or obtained from primary measurements.

Commonly, clusterin protein levels may be measured using an affinity-based measurement technology. “Affinity” as relates to an antibody is a term well understood in the art and means the extent, or strength, of binding of antibody to the binding partner, such as a biomarker as described herein (or epitope thereof). Affinity may be measured and/or expressed in a number of ways known in the art, including, but not limited to, equilibrium dissociation constant (K_{D} or K_{d}), apparent equilibrium dissociation constant (K_{D}′ or K_{d}′), and IC₅₀ (amount needed to effect 50% inhibition in a competition assay; used interchangeably herein with “I₅₀”). It is understood that, for purposes of this invention, an affinity is an average affinity for a given population of antibodies which bind to an epitope.

Affinity-based measurement technology utilizes a molecule that specifically binds to the clusterin protein being measured (an “affinity reagent,” such as an antibody or aptamer), although other technologies, such as spectroscopy-based technologies (e.g., matrix-assisted laser desorption ionization-time of flight, MALDI-TOF spectroscopy) or assays measuring bioactivity (e.g., assays measuring mitogenicity of growth factors) may be used. Affinity-based technologies may include antibody-based assays (immunoassays) and assays utilizing aptamers (nucleic acid molecules which specifically bind to other molecules), such as ELONA. Additionally, assays utilizing both antibodies and aptamers are also contemplated (e.g., a sandwich format assay utilizing an antibody for capture and an aptamer for detection).

Immunoassay technology may include any immunoassay technology which can quantitatively or qualitatively measure the level of the clusterin protein in a biological sample. Suitable immunoassay technology includes, but is not limited to radioimmunoassay, immunofluorescent assay, enzyme immunoassay, chemiluminescent assay, ELISA, immuno-PCR, and western blot assay. Likewise, aptamer-based assays which can quantitatively or qualitatively measure the level of a biomarker in a biological sample may be used in the methods of the invention. Generally, aptamers may be substituted for antibodies in nearly all formats of immunoassay, although aptamers allow additional assay formats (such as amplification of bound aptamers using nucleic acid amplification technology such as PCR (U.S. Pat. No. 4,683,202) or isothermal amplification with composite primers (U.S. Pat. Nos. 6,251,639 and 6,692,918).

Any immunoassay techniques commonly known in the art can be used in the systems and methods as disclosed herein, and include, for example, radioimmunoassay, ELISA (enzyme-linked immunosorbant assay), “sandwich” immunoassays, immunoradiometric assays, immunodiffusion assays, in situ immunoassays (using colloidal gold, enzyme or radioisotope labels, for example), western blot analysis, immunoprecipitations, immunofluorescence assays, immunoelectrophoresis assays, fluoroimmunoassay (FiA), immunoradiometric assay (IRMA), immunoenzymometric assay (IEMA), immunoluminescence assay and immunofluorescence assay (Madersbacher S, Berger P. Antibodies and immunoassays. Methods 2000; 21:41-50).

A wide variety of affinity-based assays are also known in the art. Affinity-based assays will utilize at least one epitope derived from the clusterin protein, and many affinity-based assay formats utilize more than one epitope (e.g., two or more epitopes are involved in “sandwich” format assays; at least one epitope is used to capture the clusterin protein, and at least one different epitope is used to detect the marker).

Affinity-based assays may be in competition or direct reaction formats, utilize sandwich-type formats, and may further be heterogeneous (e.g., utilize solid supports) or homogenous (e.g., take place in a single phase) and/or utilize immunoprecipitation. Many assays involve the use of labeled affinity reagent (e.g., antibody, polypeptide, or aptamer); the labels may be, for example, enzymatic, fluorescent, chemiluminescent, radioactive, or dye molecules. Assays which amplify the signals from the probe are also known; examples of which are assays which utilize biotin and avidin, and enzyme-labeled and mediated immunoassays, such as ELISA and ELONA assays. For example, the biomarker concentrations from biological fluid samples may be measured by LUMINEX® assay or ELISA, as described in Example 2 and 3. Either of the biomarker or reagent specific for the biomarker can be attached to a surface and levels can be measured directly or indirectly.

In some embodiments, one can use an immunoassay to measure the level of clusterin protein in a biological sample, for example, an ELISA method to measure clusterin protein levels using methods commonly known in the art and are encompassed for use in the present invention.

In some embodiments, a method of determining the presence and/or amount of clusterin protein in a biological sample from a subject comprises performing a binding assay. Any reasonably specific binding partner can be used. Preferably the binding partner is labeled. Preferably the assay is an immunoassay, especially between clusterin and an antibody that recognizes clusterin protein, especially a labeled antibody. It can be an antibody raised against part or all of it, most preferably a monoclonal antibody or a polyclonal anti-human antiserum of high specificity for human clusterin protein.

In some embodiments, an immunoassay is carried out by measuring the extent of the protein/antibody interaction of the clusterin/antibody interaction. Any known method of immunoassay may be used. A sandwich assay or ELISA is preferred. In this method, a first antibody to the marker protein is bound to the solid phase such as a well of a plastics microtitre plate, and incubated with the sample and with a labeled second antibody specific to the protein to be assayed. Alternatively, an antibody capture assay could be used. In some embodiments, a biological test sample is allowed to bind to a solid phase, and the anti-clusterin protein antibody is then added and allowed to bind. After washing away unbound material, the amount of antibody bound to the solid phase is determined using a labeled second antibody, anti- to the first.

In some embodiments, a label is preferably an enzyme. The substrate for the enzyme may be, for example, color-forming, fluorescent or chemiluminescent.

In some embodiments, a binding partner, e.g. an antibody or a ligand binding to clusterin in the binding assay is preferably a labeled specific binding partner, but not necessarily an antibody. The binding partner will usually be labeled itself, but alternatively it may be detected by a secondary reaction in which a signal is generated, e.g. from another labeled substance.

Thus, the any anti-clusterin antibody can be used in the method to determine the presence and/or amount of clusterin in a biological sample, which can be used to detect the increased or decreased concentration of clusterin proteins present in a diagnostic sample. Such antibodies can be raised by any of the methods well known in the immunodiagnostics field.

The antibodies may be anti-clusterin antibodies to any biologically relevant state of the protein. Thus, for example, they could be raised against the unglycosylated form of a clusterin protein which exists in the body in a glycosylated form, against a more mature form of a precursor protein, e.g. minus its signal sequence, or against a peptide carrying a relevant epitope of the marker protein.

In some embodiments, one can use an amplified form of assay, whereby an enhanced “signal” is produced from a relatively low level of protein to be detected. One particular form of amplified immunoassay is enhanced chemiluminescent assay. Conveniently, the antibody is labeled with horseradish peroxidase, which participates in a chemiluminescent reaction with luminol, a peroxide substrate and a compound which enhances the intensity and duration of the emitted light, typically 4-iodophenol or 4-hydroxycinnamic acid.

In another embodiment, an amplified immunoassay can be used which is immuno-PCR. In this technique, the antibody is covalently linked to a molecule of arbitrary DNA comprising PCR primers, whereby the DNA with the antibody attached to it is amplified by the polymerase chain reaction. See E. R. Hendrickson et al., Nucleic Acids Research 23: 522-529 (1995). The signal is read out as before.

Accordingly, in all aspects of the present invention, the level of clusterin protein can be determined using a protein-binding agent, also referred to herein as “protein-binding entity” or an “affinity reagent” can be used, in particular, antibodies. For instance, the affinity reagents, in particular, antibodies such as anti-clusterin antibodies can be used in an immunoassay, particularly in an ELISA (Enzyme Linked Immunosorbent Assay). In embodiments where the level of clusterin protein can be measured in a biological sample using methods commonly known in the art, and including, for example but not limited to isoform-specific chemical or enzymatic cleavage of isoform proteins, immunoblotting, immunohistochemical analysis, ELISA, and mass spectrometry.

As mentioned above, level of clusterin protein can be detected by immunoassays, such as enzyme linked immunoabsorbent assay (ELISA), radioimmunoassay (RIA), Immunoradiometric assay (IRMA), Western blotting, immunocytochemistry or immunohistochemistry, each of which are described in more detail below. Immunoassays such as ELISA or RIA, which can be extremely rapid, are more generally preferred. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

One of the most common enzyme immunoassay is the “Enzyme-Linked Immunosorbent Assay (ELISA).” ELISA is a technique for detecting and measuring the concentration of an antigen using a labeled (e.g. enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

In a “sandwich ELISA”, an antibody (e.g. anti-enzyme) is linked to a solid phase (i.e. a microtiter plate) and exposed to a biological sample containing antigen (e.g. enzyme). The solid phase is then washed to remove unbound antigen. A labeled antibody (e.g. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured.

In a “competitive ELISA”, antibody is incubated with a sample containing antigen (i.e. enzyme). The antigen-antibody mixture is then contacted with a solid phase (e.g. a microtiter plate) that is coated with antigen (i.e., enzyme). The more antigen present in the sample, the less free antibody that will be available to bind to the solid phase. A labeled (e.g., enzyme linked) secondary antibody is then added to the solid phase to determine the amount of primary antibody bound to the solid phase.

In an “immunohistochemistry assay” a section of tissue is tested for specific proteins by exposing the tissue to antibodies that are specific for the protein that is being assayed. The antibodies are then visualized by any of a number of methods to determine the presence and amount of the protein present. Examples of methods used to visualize antibodies are, for example, through enzymes linked to the antibodies (e.g., luciferase, alkaline phosphatase, horseradish peroxidase, or beta-galactosidase), or chemical methods (e.g., DAB/Substrate chromagen). The sample is then analyzed microscopically, most preferably by light microscopy of a sample stained with a stain that is detected in the visible spectrum, using any of a variety of such staining methods and reagents known to those skilled in the art.

Alternatively, “radioimmunoassays” can be employed. A radioimmunoassay is a technique for detecting and measuring the concentration of an antigen using a labeled (e.g. radioactively or fluorescently labeled) form of the antigen. Examples of radioactive labels for antigens include 3H, 14C, and 125I. The concentration of antigen enzyme in a biological sample is measured by having the antigen in the biological sample compete with the labeled (e.g. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (e.g., covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

An “immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

Other techniques can be used to detect the level of clusterin protein in a biological sample can be performed according to a practitioner's preference, and based upon the present disclosure and the type of biological sample (i.e. plasma, urine, tissue sample etc). One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Detectably labeled anti-clusterin antibodies or protein binding molecules can then be used to assess the level of Clusterin protein, where the intensity of the signal from the detectable label corresponds to the amount of Clusterin protein. Levels of the amount of the clusterin protein present can also be quantified, for example by densitometry.

In one embodiment, the level of clusterin protein in a biological sample can be determined by mass spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

In particular embodiments, these methodologies can be combined with the machines, computer systems and media to produce an automated system for determining the level of clusterin protein in a biological sample and analysis to produce a printable report which identifies, for example, the level of clusterin protein in a biological sample.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. See, e.g., U.S. Pat. No. 5,118,937 (Hillenkamp et al.), and U.S. Pat. No. 5,045,694 (Beavis & Chait) which are incorporated herein by reference.

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361 which are incorporated herein by reference. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition, Skoog, Saunders College Publishing, Philadelphia, 1985; and Kirk-Othmer Encyclopedia of Chemical Technology, 4^{th} ed. Vol. 15 (John Wiley & Sons, New York 1995), pp. 1071-1094.

Detection of the level of clusterin protein will typically depend on the detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

In some embodiment of this aspect and all aspects disclosed herein, a biological sample can be monitored using radioactive labeling, in particular, to an inverse radioactive labeling, preferably with iodine isotopes. Preferably, an inverse radioactive labeling is performed using ¹²⁵I and ¹³¹I isotopes. In another embodiment, a subject, for example a human subject can be subjected to a radioactive labeling, in particular, to an inverse radioactive labeling, preferably with iodine isotopes, such as but not limited to ¹²⁵I and ¹³¹I isotopes.

In all aspects of the present invention, level of clusterin protein can be determined based on gel electrophoresis techniques, in particular SDS-PAGE (Sodium Dodecylsulfate Polyacrylamide Gel Elektrophoresis), especially two dimensional PAGE (2D-PAGE), preferably two dimensional SDS-PAGE (2D-SDS-PAGE). According to a particular example, the assay is based on 2D-PAGE, in particular, using immobilized pH gradients (IPGs) with a pH range preferably over pH 4-9.

In all aspects of the present invention, the level of clusterin protein can be determined can be using gel electrophoresis techniques, in particular, the above mentioned techniques may be combined with other protein separation methods, particularly methods known to those skilled in the art, in particular, chromatography and/or size exclusion. In all aspects of the present invention, the level of clusterin protein can be determined, if appropriate, using a combination of any of the above mentioned methods with a combination of detection methods which are well known to those skilled in the art, such as, but not limited to antibody detection and/or mass spectrometry.

In a further embodiment of all aspects of the present invention, the level of clusterin protein can be determined can be using mass spectrometry as disclose herein in the Examples, and in particular, MALDI (Matrix Assisted Laser Desorption/Ionization) and/or SELDI (Surface enhanced Laser Desorption/Ionization). In an alternative embodiment, resonance techniques, in particular, plasma surface resonance, can be used.

In some cases, it may be advantageous to achieve a separation of clusterin proteins from a heterogeneous population of proteins in a biological sample for example using a means of one of the above outlined methods before cleaving the proteins. Such a cleavage step can be performed by applying enzymes, chemicals or other suitable reagents which are known to those skilled in the art. In an alternative embodiment, one may perform a cleavage step and subsequent separation of the cleaved clusterin protein fragments, in particular, followed by, for example, measurements of the level clusterin protein using any one of the methods, kits, machines, computer systems or media as disclosed herein. In some embodiments of this aspect of the invention, the cleaved clusterin protein fragments can be labeled and, optionally separated where the protein spots which correspond to cleaved Clusterin protein fragments can be visualized by imaging techniques, for instance using the PROTEP TOPO® imaging technique.

In some embodiments, a protein-binding agents or antibodies or useful in the methods as disclosed herein bind or have affinity for clusterin protein.

In some embodiments, protein-binding moieties such as antibodies can be utilized to detect the level of clusterin protein by itself (i.e. individually), or when the clusterin exists in complex with other polypeptides, for example when it is complexed with a amyloid protein, for example, but not limited to transthyretin (TTR). Additionally, in other embodiments, protein-binding moieties such as antibodies can be utilized to detect the presence of clusterin protein when it is post-translationally modified, for example when clusterin protein is ubiquitinated. In some embodiments, protein binding moieties such as antibodies can bind to a clusterin protein individually or in a complex (e.g., complexed with one or more amyloid proteins, e.g., transthyretin (TTR) or AL), and in some embodiments a protein-binding moiety such as an antibody can be labeled with a detectable label.

In some embodiments, antibodies and protein-binding molecules are labeled. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin.

In all aspects of the present invention, the level of a clusterin protein can be determined by using immunological techniques using an anti-clusterin antibody, using common methods known by a person of ordinary skill in the art, e.g., antibody techniques such as immunohistochemistry, immunocytochemistry, FACS scanning, immunoblotting, radioimmunoassays, western blotting, immunoprecipitation, enzyme-linked immunosorbant assays (ELISA), and derivative techniques that make use of antibodies directed against the clusterin protein, or variants or derivatives thereof.

Any method to detect a clusterin protein known by a person of ordinary skill in the art are useful in the methods, kits, machines and computer systems and media as disclosed herein to detect the level of clusterin protein. For example, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of a specific antibody, wherein antibodies are used to specifically target molecules inside or on the surface of cells. The antibody typically contains a marker that will undergo a biochemical reaction, and thereby experience a change color, upon encountering the targeted molecules. In some instances, signal amplification may be integrated into the particular protocol, wherein a secondary antibody, that includes the marker stain, follows the application of a primary specific antibody. Immunohistochemical assays are well known to those of skill in the art (e.g., see Jalkanen, et al., J. Cell. Biol. 101:976-985 (1985); Jalkanen, et al., J. Cell. Biol. 105:3087-3096 (1987).

In some embodiments, antibodies, polyclonal, monoclonal and chimeric antibodies useful in the methods as disclosed herein can be purchased from a variety of commercial suppliers, or may be manufactured using well-known methods, e.g., as described in Harlow et al., Antibodies: A Laboratory Manual, 2nd Ed; Cold. Spring Harbor Laboratory Press, Cold Spring Harbor, N.Y. (1988). In general, examples of antibodies useful in the present invention include anti-serine antibodies. Such antibodies can be purchased, for example, from Sigma-Aldrich, CalBiochem, Abcam, Santa-Cruz Biotechnology, novus Bio, U.S. biologicals, Millipore, LifeSpan, Abnova, CellSignalling etc.

In some embodiments, direct labeling techniques can be used, where a labeled antibody is utilized. For indirect labeling techniques, the sample is further reacted with a labeled substance.

In some embodiments, immunocytochemistry may be utilized where, in general, tissue or cells are obtained from a subject are fixed by a suitable fixing agent such as alcohol, acetone, and paraformaldehyde, to which is reacted an antibody. Methods of immunocytological staining of human samples is known to those of skill in the art and described, for example, in Brauer et al., 2001 (FASEB J, 15, 2689-2701), Smith Swintosky et al., 1997.

Immunological methods are particularly useful in the methods as disclosed herein, because they require only small quantities of biological material, and are easily performed and at multiple different locations. In some embodiments, such an immunological method useful in the methods as disclosed herein uses a “lab-on-a-chip” device, involving a single device to run a single or multiple biological samples and requires minimal reagents and apparatus and is easily performed, making the “lab-on-a-chip” devices which detect the clusterin protein levels is ideal for rapid, on-site diagnostic tests to identify if the subject from whom the biological sample was obtained from is likely to have cardiac amyloid deposits and/or amyloidotic cardiomyopathy. In some embodiments, the immunological methods can be done at the cellular level and thereby necessitate a minimum of one cell. Preferably, several cells are obtained from a subject affected at risk for cardiac amyloid deposits, e.g., a subject with an amyloid disease, e.g., a subject with a TTR-associated disease or AL amyloidosis or the like using the methods, computer systems and computer readable media as disclosed herein

Alternatively, in some embodiments, one method to determine the amount of clusterin in a biological sample is to use a two dimensional gel electrophoresis to yield a stained gel and the increased or decreased concentration of the protein detected by an increased an increased or decreased intensity of a protein-containing spot on the stained gel, compared with a corresponding control or comparative gel.

In some embodiments, methods to determine the amount of clusterin in a biological sample does not necessarily require a step of comparison of the concentration of clusterin protein with a control sample, but it can be carried out with reference either to a control or a comparative sample. Thus, in relation to TTR amyloidosis or amyloidotic cardiomyopathy, measuring the amount of clusterin in a biological sample can be used to determine the stage of progression, if desired with reference to results obtained earlier from the same subject or by reference to standard values that are considered typical of the stage of the disease. In this way, the invention can be used to determine whether, for example after treatment of the subject with clusterin protein or clusterin agent, the disease has progressed or not. The result can lead to a prognosis of the outcome of the disease.

In a heterogeneous format, the assay utilizes two phases (typically aqueous liquid and solid). Typically a biomarker-specific affinity reagent is bound to a solid support to facilitate separation of the biomarker from the bulk of the biological sample. After reaction for a time sufficient to allow for formation of affinity reagent/biomarker complexes, the solid support or surface containing the antibody is typically washed prior to detection of bound polypeptides. The affinity reagent in the assay for measurement of biomarkers may be provided on a support (e.g., solid or semi-solid); alternatively, the polypeptides in the sample can be immobilized on a support or surface. Examples of supports that can be used are nitrocellulose (e.g., in membrane or microtiter well form), polyvinyl chloride (e.g., in sheets or microtiter wells), polystyrene latex (e.g., in beads or microtiter plates), polyvinylidine fluoride, diazotized paper, nylon membranes, activated beads, glass and Protein A beads. Both standard and competitive formats for these assays are known in the art. Accordingly, provided herein are complexes comprising clusterin bound to a reagent specific for the biomarker, wherein said reagent is attached to a surface. Also provided herein are complexes comprising at least one biomarker bound to a reagent specific for the biomarker, wherein said biomarker is attached to a surface.

Array-type heterogeneous assays are suitable for measuring the level of clusterin protein when the methods of the invention are practiced in utilizing multiple samples or levels of clusterin protein are measured with levels of other biomarker proteins. Array-type assays used in the practice of the methods of the invention will commonly utilize a solid substrate with two or more capture reagents specific for clusterin and different biomarkers bound to the substrate a predetermined pattern (e.g., a grid). A biological fluid sample is applied to the substrate and biomarkers in the sample are bound by the capture reagents. After removal of the sample (and appropriate washing), the bound biomarkers are detected using a mixture of appropriate detection reagents that specifically bind the various biomarkers. Binding of the detection reagent is commonly accomplished using a visual system, such as a fluorescent dye-based system. Because the capture reagents are arranged on the substrate in a predetermined pattern, array-type assays provide the advantage of detection of multiple biomarkers without the need for a multiplexed detection system.

In a homogeneous format the assay takes place in single phase (e.g., aqueous liquid phase). Typically, the biological sample is incubated with an affinity reagent specific for the clusterin protein in solution. For example, it may be under conditions that will precipitate any affinity reagent/antibody complexes which are formed. Both standard and competitive formats for these assays are known in the art.

In a standard (direct reaction) format, the level of clusterin/affinity reagent complex is directly monitored. This may be accomplished by, for example, determining the amount of a labeled detection reagent that forms is bound to clusterin/affinity reagent complexes. In a competitive format, the amount of clusterin protein in the sample is deduced by monitoring the competitive effect on the binding of a known amount of labeled clusterin (or other competing ligand) in the complex. Amounts of binding or complex formation can be determined either qualitatively or quantitatively.

The methods described in this patent may be implemented using any device capable of implementing the methods. Examples of devices that may be used include but are not limited to electronic computational devices, including computers of all types. When the methods described in the present invention are implemented in a computer, the computer program that may be used to configure the computer to carry out the steps of the methods may be contained in any computer readable medium capable of containing the computer program. Examples of computer readable medium that may be used include but are not limited to diskettes, CDROMs, DVDs, ROM, RAM, and other memory and computer storage devices. The computer program that may be used to configure the computer to carry out the steps of the methods may also be provided over an electronic network, for example, over the internet, world-wide web, an intranet, or other network.

In one example, the methods described in the present invention may be implemented in a system comprising a processor and a computer readable medium that includes program code means for causing the system to carry out the steps of the methods described in the present invention. The processor may be any processor capable of carrying out the operations needed for implementation of the methods. The program code means may be any code that when implemented in the system can cause the system to carry out the steps of the methods described in the present invention. Examples of program code means include but are not limited to instructions to carry out the methods described in this patent written in a high level computer language such as C++, Java, or Fortran; instructions to carry out the methods described in the present invention written in a low level computer language such as assembly language; or instructions to carry out the methods described in the present invention in a computer executable form such as compiled and linked machine language.

Complexes comprising clusterin and an affinity reagent can be detected by any of a number of known techniques known in the art, depending on the format of the assay and the preference of the user. For example, unlabelled affinity reagents may be detected with DNA amplification technology (e.g., for aptamers and DNA-labeled antibodies) or labeled “secondary” antibodies which bind the affinity reagent. Alternately, the affinity reagent may be labeled, and the amount of complex may be determined directly (as for dye-(fluorescent or visible), bead-, or enzyme-labeled affinity reagent) or indirectly (as for affinity reagents “tagged” with biotin, expression tags, and the like).

As will be understood by those of skill in the art, the mode of detection of the signal will depend on the detection system utilized in the assay. For example, if a radiolabeled detection reagent is utilized, the signal will be measured using a technology capable of quantitation of the signal from the biological sample or of comparing the signal from the biological sample with the signal from a reference sample, such as scintillation counting, autoradiography (typically combined with scanning densitometry), and the like. If a chemiluminescent detection system is used, then the signal will typically be detected using a luminometer. Methods for detecting signal from detection systems are well known in the art and need not be further described here.

When clusterin levels are to be measured multiple times, or at different intervals, a biological sample may be divided into a number of aliquots, with separate aliquots used to clusterin levels at different concentrations and/or times (although division of the biological sample into multiple aliquots to allow multiple determinations of clusterin levels in a particular sample are also contemplated). Alternately the biological sample (or an aliquot therefrom) may be tested to determine the levels of clusterin protein in a single reaction using an assay capable of measuring the individual levels of clusterin in a single assay, such as an array-type assay or assay utilizing multiplexed detection technology (e.g., an assay utilizing detection reagents labeled with different fluorescent dye markers).

It is common in the art to perform “replicate” measurements when measuring clusterin levels. Replicate measurements are ordinarily obtained by splitting a sample into multiple aliquots, and separately measuring the clusterin protein levels in separate reactions of the same assay system. Replicate measurements are not necessary to the methods of the invention, but many embodiments of the invention will utilize replicate testing, particularly duplicate and triplicate testing.

### Reference Values and Control Subject

The reference values of clusterin levels used for comparison with the level of clusterin measured from a subject may vary, depending on the aspect of the invention being practiced, as will be understood throughout this specification, and below. A reference value, e.g., reference clusterin level, can be based on an individual sample value, such as for example, a value obtained from a biological sample from the subject being tested, but at an earlier point in time (e.g., at a first timepoint (t1), e.g., a first clusterin level measured, or at a second timepoint (t2), e.g.,). A reference value, e.g., reference clusterin level, can also be based on a pool of samples, for example, value(s) obtained from samples from a pool of subjects being tested. Reference value(s) can also be based on a pool of samples including or excluding the sample(s) to be tested. The reference value can be based on a large number of samples, such as from population of healthy subjects of the chronological age-matched group, or from subjects such as TTR subjects who do not have amyloidotic cardiomyopathy (CMP).

For assessing the risk of amyloidotic cardiomyopathy (CMP) in a subject by the methods and systems as disclosed herein, a “reference value” is typically a predetermined reference level, such as an average or median of levels of clusterin protein obtained from a population of healthy subjects that are in the chronological age group matched with the chronological age of the tested subject. As indicated earlier, in some situations, the reference samples may also be gender matched.

For assessing the risk of amyloidotic cardiomyopathy (CMP) in a subject by the methods and systems as disclosed herein, the reference clusterin level may be a predetermined level, such as an average or median of levels obtained from a population of healthy subjects that are in the chronological age group matched with the chronological age of the tested subject. In some embodiments, such a predetermined level of a reference clusterin level is about 0.6 mg/mL, or between about 0.5 mg/mL and about 0.7 mg/mL. Alternately, the reference clusterin level may be a historical reference level for the particular subject (e.g., a clusterin protein level that was obtained from a sample derived from the same subject, but at an earlier point in time, and/or when the subject did not have cardiac amyloid deposits). In some instances, the reference clusterin level may be a historical reference level of clusterin protein for the particular groups of subjects (e.g., clusterin protein levels that were obtained from samples derived from the same group of subjects, but at an earlier point in time).

In some embodiments, control subjects are ischemic and non-ischemic CMP patients who do not have amyloidosis (e.g., amyloid negative controls) or amyloidosis patients with no cardiac involvement.

In some embodiments, healthy subjects are selected as the control subjects. In some embodiments, controls are age-matched controls. Healthy subject may be used to obtain a reference level of clusterin protein, e.g., clusterin protein in a serum sample. A “healthy” subject or sample from a “healthy” subject or individual as used herein is the same as those commonly understood to one skilled in the art. For example, one may use methods commonly known to evaluate cardiac function, and/or amyloidosis to select control subjects as healthy subjects for diagnosis and treatment methods related to amyloidic cardiomyopathy. In some embodiments, subjects in good health with no signs or symptom suggesting cardiac dysfunction or an amyloid disease are recruited as healthy control subjects. The subjects are evaluated based on extensive evaluations consisted of medical history, family history, physical and cardiac examinations by clinicians who cardiology and/or amyloid diseases, laboratory tests. Examples of analysis of cardiac function and cardiac amyloid disease include, but are not limited to (i) electrocardiogram (ECG or EKG) which is a graphic recordation of cardiac activity, either on paper or a computer monitor. An ECG can be beneficial in detecting disease and/or damage; (ii) echocardiogram (heart ultrasound) used to investigate congenital heart disease and assessing abnormalities of the heart wall, including functional abnormalities of the heart wall, valves and blood vessels; (iiii) Doppler ultrasound (or Doppler imaging (TDI) and strain imaging (SI)) can be used to measure blood flow across a heart valve; (iv) nuclear medicine imaging (also referred to as radionuclide scanning in the art) allows visualization of the anatomy and function of an organ, and (v) magnetic resonance imaging (MRI) can be used to detect presence of amyloid deposits on organs, including the heart. In some embodiments, a control subject can be selected by lack of congo red staining or lack of anti-mycin staining of endomyocardia biopsy samples. Other methods to identify lack of cardiac amyloid deposits are known, for example, traditional echocardiographic techniques as well as new echocardiographic imaging modalities such as tissue Doppler, Doppler-based strain, speckle tracking imaging, and three-dimensional imaging in the assessment of cardiac amyloid (as disclosed in Tsang et al., Echocardiographic Evaluation of Cardiac Amyloid, Curr Cardiology Reports, 2010, 12(3), 272-276).

Age-matched populations (from which reference values may be obtained) are ideally the same chronological age as the subject or individual being tested, but approximately age-matched populations are also acceptable. Approximately age-matched populations may be within 1, 2, 3, 4, or 5 years of the chronological age of the individual tested, or may be groups of different chronological ages which encompass the chronological age of the individual being tested.

A subject that is compared to its “chronological age matched group” is generally referring to comparing the subject with a chronological age-matched within a range of 5 to 20 years. Approximately age-matched populations may be in 2, 3, 4, 5, 6, 7, 8, 9, 10 or 15, or 20 year increments (e.g. a “5 year increment” group may serve as the source for reference values for a 62 year old subject might include 58-62 year old individuals, 59-63 year old individuals, 60-64 year old individuals, 61-65 year old individuals, or 62-66 year old individuals). In a broader definition, where there are larger gaps between different chronological age groups, for example, when there are few different chronological age groups available for reference values, and the gaps between different chronological age groups exceed the 2, 3, 4, 5, 6, 7, 8, 9, 10 or 15, or 20 year increments described herein, then the “chronological age matched group” may refer to the age group that is in closer match to the chronological age of the subject (e.g. when references values available for an older age group (e.g., 80-90 years) and a younger age group (e.g., 20-30 years), a chronological age matched group for a 51 year old may use the younger age group (20-30 years), which is closer to the chronological age of the test subject, as the reference level.

Other factors to be considered while selecting control subjects include, but not limited to, species, gender, ethnicity, and so on. Moreover, clusterin levels may be different within different age groups, and/or may be gender specific. Hence in one embodiment, a reference level may be a predetermined reference level, such as an average or median of levels obtained from a population of healthy control subjects that are gender-matched with the gender of the tested subject, e.g., where such a predetermined level of a reference clusterin level is about 0.6 mg/mL, or between about 0.5 mg/mL-0.7 mg/mL. In some embodiments, a reference level may be a predetermined reference level, such as an average or median of levels obtained from a population of healthy control subjects that are ethnicity-matched with the ethnicity of the tested subject. In another embodiment, both chronological age and gender of the population of healthy subjects are matched with the chronological age and gender of the tested subject, respectively. In another embodiment, both chronological age and ethnicity of the population of healthy subjects are matched with the chronological age and ethnicity of the tested subject, respectively. In a further embodiment, chronological age, gender, and ethnicity of the population of healthy control subjects are all matched with the chronological age, gender, and ethnicity of the tested subject, respectively.

### Comparing Levels of Biomarkers

The process of comparing a level of clusterin protein in a biological sample from a subject and a reference clusterin level can be carried out in any convenient manner appropriate. Generally, values of clusterin protein levels used in the methods of the invention may be quantitative values (e.g., quantitative values of concentration, such as milligrams of clusterin per milliliter (e.g., mg/mL) of sample, or an absolute amount). Alternatively, values of clusterin protein level can be qualitative depending on the measurement techniques, and thus the mode of comparing a value from a subject and a reference value can vary depending on the measurement technology employed. For example, the comparison can be made by inspecting the numerical data, by inspecting representations of the data (e.g., inspecting graphical representations such as bar or line graphs). In one example, when a qualitative calorimetric assay is used to measure clusterin levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device).

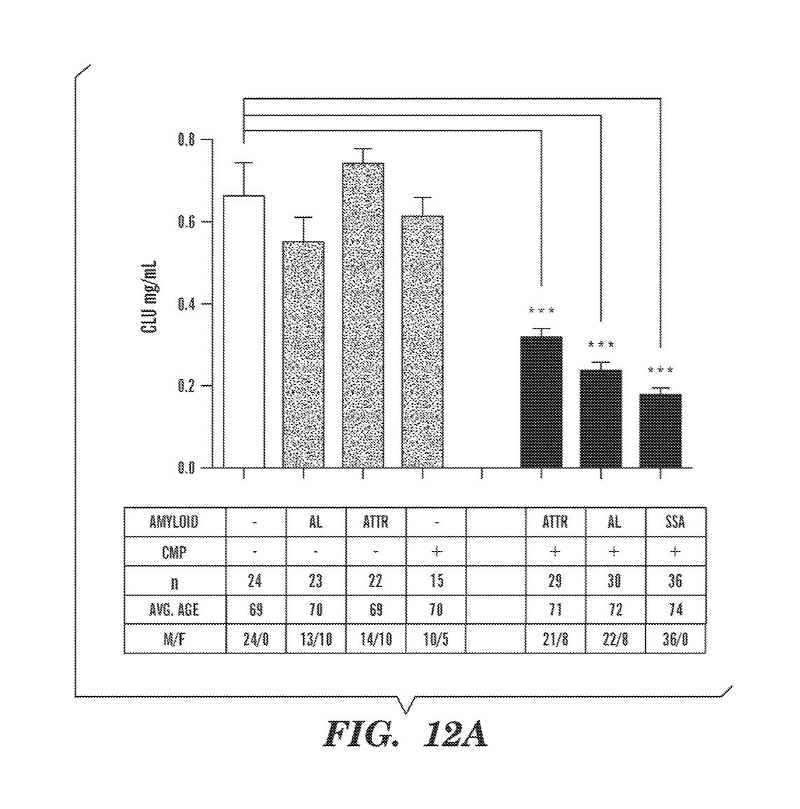

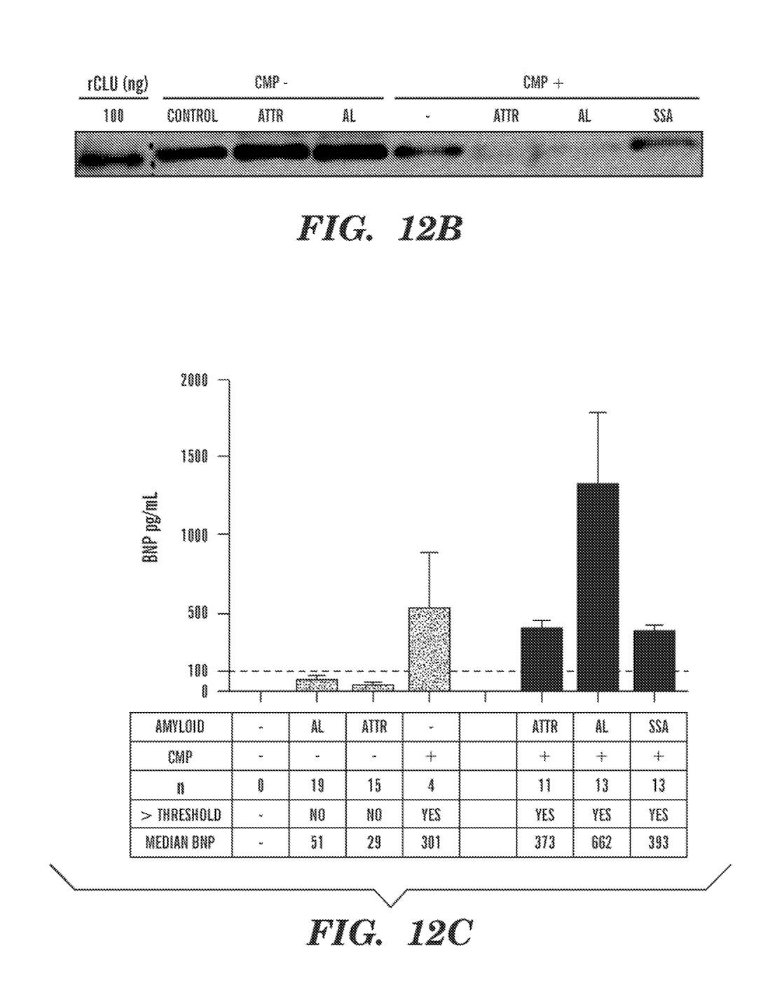

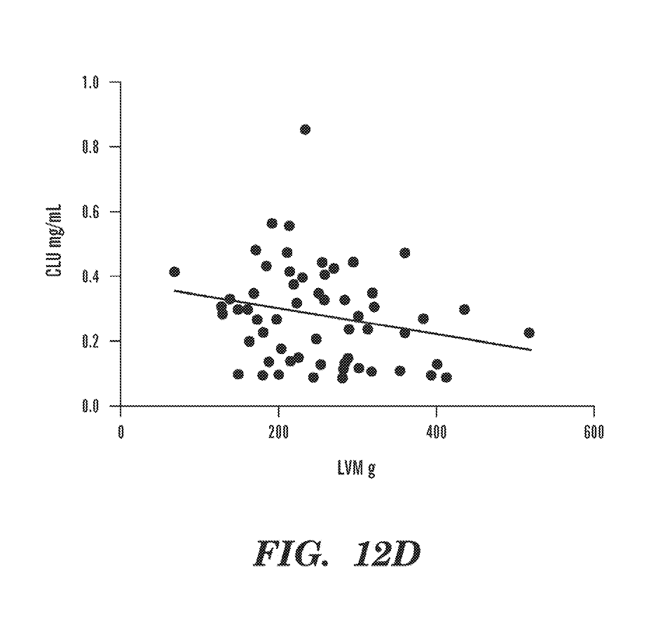

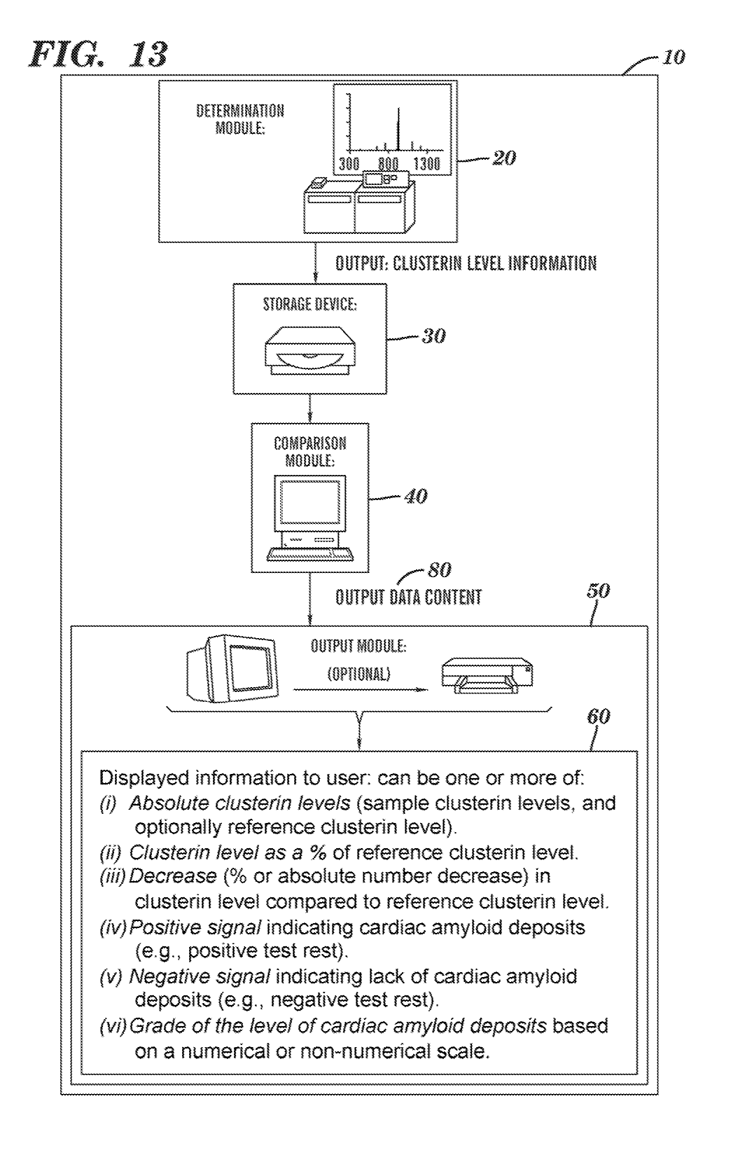

As described herein, biological fluid samples may be measured quantitatively (absolute values) or qualitatively (relative values). In some embodiments, quantitative values of clusterin levels in the biological fluid samples may indicate a given level (or grade) of cardiac amyloid deposits and/or amyloidotic cardiomyopathy (CMP). For example, quantitative values of biomarkers in the biological fluid samples may indicate a given level of cardiac amyloid deposits or left ventricle mass (LVM). As shown in Example 4 and FIG. 12D, there is an inverse correlation with the low levels of clusterin protein in serum and higher LVM, demonstrating that lower levels of clusterin protein is strongly indicative of high left ventricle mass (LVM). Hence quantitative values, such as concentrations of clusterin protein, can be used to compare the concentration of clusterin from a subject to a reference clusterin protein concentration of the biomarker to diagnosis and/or monitor the progress of cardiac amyloid deposits and/or amyloidotic cardiomyopathy (CMP).

In certain embodiments, the comparison is performed to determine the magnitude of the difference between the values from a subject and reference values (e.g., comparing the “fold” or percentage difference between the clusterin protein value from a subject and the reference clusterin value). A decrease in the fold of about one third (e.g., about a 0.333-fold decrease, e.g., about a 33% lower value below) of the clusterin levels as compared to the reference clusterin level indicates a diagnosis of cardiac amyloid deposits and/or amyloidotic cardiomyopathy (CMP). A fold difference can be determined by measuring the absolute concentration of a clusterin protein and comparing that to the absolute value to the reference clusterin level, or a fold difference can be measured by the relative difference between a reference value and a sample value, where neither value is a measure of absolute concentration, and/or where both values are measured simultaneously. For example, an ELISA measures the absolute content or concentration of a protein from which a fold change is determined in comparison to the absolute concentration of the same protein in the reference. As another example, an antibody array measures the relative concentration from which a fold change is determined. Accordingly, the magnitude of the difference between the measured value and the reference value that suggests or indicates a particular diagnosis will depend on the particular biomarker being measured to produce the measured value and the reference value used (which in turn depends on the method being practiced).

As will be apparent to those of skill in the art, when replicate measurements are taken for measurement of clusterin protein levels, the value from a subjected measured that is compared with the reference clusterin protein value is a value that takes into account the replicate measurements. The replicate measurements may be taken into account by using either the mean or median of the measured values.

In some embodiments, the process of comparing may be manual (such as visual inspection by the practitioner of the method) or it may be automated. For example, an assay device (such as a luminometer for measuring chemiluminescent signals) may include circuitry and software enabling it to compare a value from a subject with a reference value for a biomarker. Alternately, a separate device (e.g., a digital computer) may be used to compare the clusterin level value(s) from subject(s) and the reference clusterin level value(s). Automated devices for comparison may include stored reference values for the clusterin protein being measured, or they may compare the clusterin level value(s) from subject(s) with reference clusterin values that are derived from contemporaneously measured reference samples.

Methods of Diagnosing, Treating and Monitoring Cardiac Amyloid Deposits and/or Amyloidotic Cardiomyopathy (CMP)

One aspect of the present invention relates to methods and systems for diagnosing a subject with cardiac amyloid deposits and/or amyloidotic cardiomyopathy in a biological sample from a subject, the method comprising measuring the level of clusterin protein in a biological sample obtained from the subject, wherein a decrease in the level of clusterin protein in the biological sample a statistically significant amount as compared to a reference level of clusterin protein is indicative of the subject being at risk of having cardiac amyloid deposits. In some embodiments, the cardiac amyloid deposits and/or amyloidotic cardiomyopathy is associated with a TTR amyloidosis, including but not limited to familial amyloidosis, e.g. senile systemic amyloidosis (SSA), familial amyloid polyneuropathy (FAP), or familial amyloidotic cardiomyopathy (FAC). The subject may be human or the subject can be non-human.

Another aspect of the present invention provides a method to treat a subject identified to have amyloidotic cardiomyopathy using the methods, systems and kits as disclosed herein, the method comprising administering to a subject for the treatment of disease or disorder associated with amyloidosis an effective amount of a clusterin protein or an agonist of clusterin activity.

### Systems and Computer Readable Media

One aspect of the present invention relates to a system for assessing if a subject has cardiac amyloid deposits, the system 10 as shown as an exemplary example in FIG. 13 comprises: (a) a determination module 20 configured to receive a biological sample, measure levels of clusterin protein in the biological sample and to output information of the level of clusterin protein in the biological sample; (b) a storage device 30 configured to store clusterin level output information from the determination module; (c) a comparison module 40 adapted to receive input from the storage device and compare the data stored on the storage device with at least one reference clusterin level data, wherein if the reference clusterin level data is 1.5-fold or more higher than the input clusterin protein level information, the comparison module provides information to an output module that the biological sample is associated with a subject that deviates from the reference clusterin protein level; and (d) an output module 50 for displaying the information 60 to the user. In some embodiments, a 1.5-fold increase of the reference clusterin level as compared to the measured clusterin level corresponds to about a 25% decrease in the measured clusterin level as compared to the reference clusterin level. Accordingly, if the reference clusterin level is about 2-fold higher than the measured clusterin level, it represents about a 50% decrease in the clusterin level as compared to the reference clusterin level.

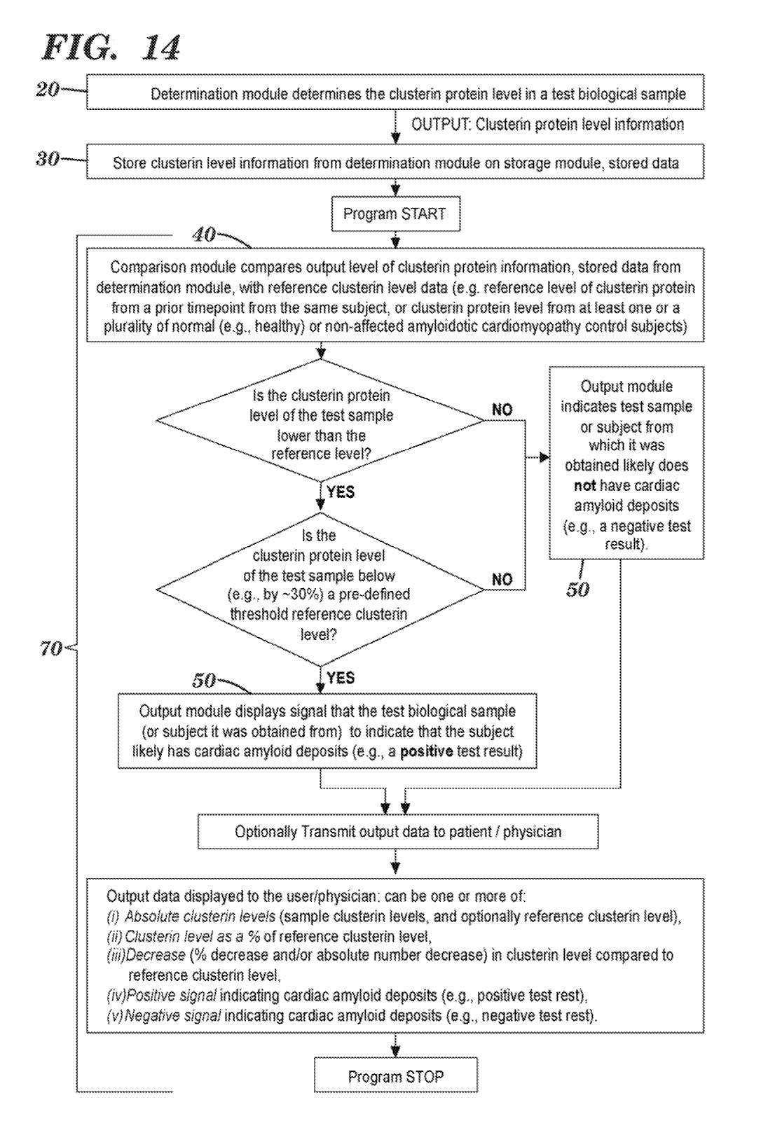

Another aspect relates to a computer readable media, or computer readable physical storage media having instructions 70 recorded thereon sufficient to implement the system 10 as disclosed herein on a computer processor. In some embodiments, as shown in FIG. 14, the computer readable media instructs the computer to carry out a process, where the instructions comprising: a) instructions for receiving data regarding the clusterin protein expression in a biological sample, b) instructions for comparing the level of said clusterin protein level in said biological sample with a reference clusterin protein level, and c) instructions for transmitting to a user interface the clusterin levels from the biological sample, and optionally the reference clusterin levels. In some embodiments, the computer readable media provides instructions to optionally transmit a result of said comparison, e.g., the instructions can direct the transmission to provide the results of the comparison, wherein a decrease in the level of clusterin protein in said biological sample from the subject as compared to a reference clusterin protein level indicates a subject is likely to have a cardiac amyloid deposits.

Computer-readable physical storage media useful in various embodiments include any physical computer-readable storage medium, e.g., magnetic and optical computer-readable storage media, among others. Carrier waves and other signal-based storage or transmission media are not included within the scope of physical computer-readable storage media encompassed by the term and useful according to the invention.

A user interface useful in various embodiments includes, for example, a display screen or a printer or other means for providing a readout of the result of a computer-mediated process. A user interface can also include, for example, an address in a network or on the world wide web to which the results of a process are transmitted and made accessible to one or more users. For example, the user interface can include a graphical user interface comprising an access element that permits entry of data regarding clusterin levels in a biological sample, as well as an access element that provides a graphical read out of the results of a comparison transmitted to or made available by a processor following execution of the instructions encoded on a computer-readable medium.

Embodiments of the invention also provide for systems 10 (and computer readable medium with instructions 70 for causing computer systems) to perform a method for determining the levels of clusterin in a biological sample from a subject, or alternatively if a subject has, or has increased risk of having cardiac amyloid deposits, and/or amyloidotic cardiomyopathy.

Embodiments of the invention have been described through functional modules, which are defined by computer executable instructions 70 recorded on computer readable media and which cause a computer to perform method steps when executed. The modules have been segregated by function for the sake of clarity. However, it should be understood that the modules need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable media can be any available tangible media that can be accessed by a computer. Computer readable media includes volatile and nonvolatile, removable and non-removable tangible media implemented in any method or technology for storage of information such as computer readable instructions, data structures, program modules or other data. Computer readable media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media, or computer readable medium, may define instructions, for example, as part of one or more programs, that, as a result of being executed by a computer, instruct the computer to perform one or more of the functions described herein and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of system, or computer readable medium described herein, may be distributed across one or more of such components, and may be in transition there between.

The computer-readable media may be transportable such that the instructions 70 stored thereon can be loaded onto any computer resource to implement the aspects of the present invention discussed herein. In addition, it should be appreciated that the instructions 70 stored on the computer readable media, or the computer-readable medium are not limited to instructions embodied as part of an application program running on a host computer. Rather, the instructions may be embodied as any type of computer code (e.g., software or microcode) that can be employed to program a computer to implement aspects of the present invention. The computer executable instructions 70 may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The functional modules of certain embodiments of the invention include a determination module 20, a storage device 30, a comparison module 40 and an output module 50. In some embodiments, the output module 40 is a display module. The functional modules can be executed on one, or multiple, computers, or by using one, or multiple, computer networks. The determination module can have computer executable instructions to measure clusterin protein level information in computer readable form. As used herein, “clusterin protein level information” refers to the level of clusterin protein, preferably clusterin protein level of the polypeptide of SEQ ID NO: 1 and fragments and variants thereof. The term “clusterin protein level information” is intended to include the presence or absence of post-translational modifications (e.g. phosphorylation, glycosylation, summylation, farnesylation, and the like) of the measured clusterin protein, as well as clusterin complexed with other proteins, e.g., amyloid proteins such as TTR.

As an example, a determination module 20 for determining level of clusterin protein information may include known systems for automated protein expression level determination, including for example, but not limited to, mass spectrometry systems including Matrix Assisted Laser Desorption Ionization-Time of Flight (MALDI-TOF) systems and SELDI-TOF-MS ProteinChip array profiling systems; systems for analyzing gene expression data (see, for example, published U.S. Patent Application, Pub. No. U.S. 2003/0194711, which is incorporated herein in its entirety by reference); systems for array based expression analysis: e.g., HT array systems and cartridge array systems such as GENECHIP® AUTOLOADER, COMPLETE GENECHIP® Instrument System, GENECHIP® Fluidics Station 450, GENECHIP® Hybridization Oven 645, GENECHIP® QC Toolbox Software Kit, GENECHIP® Scanner 3000 7G plus Targeted Genotyping System, GENECHIP® Scanner 3000 7G Whole-Genome Association System, GENETITAN™ Instrument, and GENECHIP® Array Station (each available from Affymetrix, Santa Clara, Calif.); automated ELISA systems (e.g., DSX® or DS2® (available from Dynax, Chantilly, Va.) or the TRITURUS® (available from Grifols USA, Los Angeles, Calif.), The MAGO® Plus (available from Diamedix Corporation, Miami, Fla.); Densitometers (e.g. X-Rite-508-SPECTRO DENSITOMETER® (available from RP IMAGING™, Tucson, Ariz.), The HYRYS™ 2 HIT densitometer (available from Sebia Electrophoresis, Norcross, Ga.); automated Fluorescence insitu hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, (available from Becton Dickinson, Franklin Lakes, N.J.); and radio isotope analyzers (e.g. scintillation counters).

In some embodiments, the determination module 20 has computer executable instructions to provide information in computer readable form. As an example, a determination module 20 for determining the level of clusterin protein by binding of a protein-binding molecule to a protein, for example but not limited to the binding of an anti-clusterin antibody to a clusterin protein include for example but are not limited to automated immunohistochemistry apparatus, for example, robotically automated immunohistochemistry apparatus which in an automated system section the tissue or biological sample specimen, prepare slides, perform immunhistochemistry procedure and detect intensity of immunostaining, such as intensity of anti-clusterin antibody staining in the biological sample or tissue and produce output data. Examples of such automated immunohistochemistry apparatus are commercially available, for example such Autostainers 360, 480, 720 and Labvision PT module machines from LabVision Corporation, which are disclosed in U.S. Pat. Nos. 7,435,383; 6,998,270; 6,746,851, 6,735,531; 6,349,264; and 5,839; 091 which are incorporated herein in their entirety by reference. Other commercially available automated immunohistochemistry instruments are also encompassed for use in the present invention, for example, but not are limited BOND™ Automated Immunohistochemistry & In Situ Hybridization System, Automate slide loader from GTI vision. Automated analysis of immunohistochemistry can be performed by commercially available systems such as, for example, IHC Scorer and Path EX, which can be combined with the Applied spectral Images (ASI) CytoLab view, also available from GTI vision or Applied Spectral Imaging (ASI) which can all be integrated into data sharing systems such as, for example, Laboratory Information System (LIS), which incorporates Picture Archive Communication System (PACS), also available from Applied Spectral Imaging (ASI) (see world-wide-web: spectral-imaging.com). Other a determination module can be an automated immunohistochemistry systems such as NexES® automated immunohistochemistry (IHC) slide staining system or BenchMark®LT automated IHC instrument from Ventana Discovery SA, which can be combined with VIAS™ image analysis system also available Ventana Discovery. BioGenex Super Sensitive MultiLink® Detection Systems, in either manual or automated protocols can also be used as the detection module, preferably using the BioGenex Automated Staining Systems. Such systems can be combined with a BioGenex automated staining systems, the i6000™ (and its predecessor, the OptiMax® Plus), which is geared for the Clinical Diagnostics lab, and the GenoMx 6000™, for Drug Discovery labs. Both systems BioGenex systems perform “All-in-One, All-at-Once” functions for cell and tissue testing, such as Immunohistochemistry (IHC) and In Situ Hybridization (ISH).

As an example, a determination module 20 for determining (e.g., measuring) the level of clusterin protein may include known systems for automated protein expression analysis including but not limited Mass Spectrometry systems including MALDI-TOF, or Matrix Assisted Laser Desorption Ionization-Time of Flight systems; SELDI-TOF-MS ProteinChip array profiling systems, e.g. Machines with Ciphergen Protein Biology System II™ software; systems for analyzing gene expression data (see for example U.S. 2003/0194711); systems for array based expression analysis, for example HT array systems and cartridge array systems available from Affymetrix (Santa Clara, Calif. 95051) AutoLoader, Complete GeneChip® Instrument System, Fluidics Station 450, Hybridization Oven 645, QC Toolbox Software Kit, Scanner 3000 7G, Scanner 3000 7G plus Targeted Genotyping System, Scanner 3000 7G Whole-Genome Association System, GeneTitan™ Instrument, GeneChip® Array Station, HT Array; an automated ELISA system (e.g. DSX® or DS2® form Dynax, Chantilly, Va. or the ENEASYSTEM III®, Triturus®, The Mago® Plus); Densitometers (e.g. X-Rite-508-Spectro Densitometer®, The HYRYS™ 2 densitometer); automated Fluorescence insitu hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, Becton Dickinson); radio isotope analyzers (e.g. scintillation counters).

Algorithms for identifying protein expression levels and profiles, such as the total amount of clusterin available in a biological sample can include the use of optimization algorithms such as the mean variance algorithm, e.g. JMP Genomics algorithm available from JMP Software.

In some embodiments of this aspect and all other aspects of the present invention a variety of software programs and formats can be used to store the clusterin protein level information on the storage device. Any number of data processor structuring formats (e.g., text file or database) can be employed to obtain or create a medium having recorded thereon the sequence information or expression level information.

The level of clusterin protein information determined in the determination module 20 can be read by the storage device 30. As used herein the “storage device” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media.

Storage devices 30 are also commonly referred to in the art as “computer-readable physical storage media” which is useful in various embodiments, and can include any physical computer-readable storage medium, e.g., magnetic and optical computer-readable storage media, among others. Carrier waves and other signal-based storage or transmission media are not included within the scope of storage devices or physical computer-readable storage media encompassed by the term and useful according to the invention. The storage device is adapted or configured for having recorded thereon cytokine level information. Such information may be provided in digital form that can be transmitted and read electronically, e.g., via the Internet, on diskette, via USB (universal serial bus) or via any other suitable mode of communication.

As used herein, “level of clusterin protein” or “clusterin protein level information” refers to any amino acid expression level information of at least one clusterin isoform, including but not limited to full-length amino acid sequence of clusterin such as SEQ ID NO: 1, as well as partial amino acid sequences, or mutated sequences.

As used herein, “stored” refers to a process for encoding information on the storage device. Those skilled in the art can readily adopt any of the presently known methods for recording information on known media to generate manufactures comprising information of the level of clusterin.

A variety of software programs and formats can be used to store the information of level of clusterin protein on the storage device. Any number of data processor structuring formats (e.g., text file or database) can be employed to obtain or create a medium having recorded thereon the information comprising the level of clusterin protein.

By providing the level of clusterin protein in computer-readable form, one can use the information comprising the level of clusterin protein on in readable form in a comparison module 40 to compare a specific clusterin protein level from the biological sample with the reference clusterin level data, e.g., where the reference level can also be stored within the storage device 30. For example, search programs can be used to identify which reference clusterin level data (for example, age-matched, gender matched, and comparable control population) should be used to compare with the clusterin level measured from the biological sample, or alternatively, a direct comparison of the determined clusterin level from the biological sample measured by the determination module can be compared to a reference clusterin data obtained from the same subject at one or more earlier timepoints (e.g., clusterin level information was obtained from the subject to be used as a reference clusterin level data). Typically, a comparison can be made with an electronic computer in computer-readable form, where the computer can follow instructions 70 and provides a computer readable comparison result which can be processed by a variety of means. Content based on the comparison result can be retrieved from the comparison module 40 by the output module 50 to indicate, for example, at least one of the following; (i) the presence or absence of cardiac amyloid deposits, (ii) the level of clusterin protein in the biological sample obtained from the subject, (iii) and in some embodiments, the level of clusterin protein in the biological sample obtained from the subject and the reference clusterin level, or optionally, in some embodiments a signal to indicate the subject has, or is likely to have cardiac amyloid deposits.

In some embodiments, the comparison module 40 provides information to an output module 50, wherein the output module can transmit the information to an end user, e.g., via a network e.g., world wide web, or produce a report, e.g., printed report, where the output information 60 displayed to the user can include one or more of the following information: (a) absolute clusterin levels (e.g., mg/ml clusterin levels in the biological sample, and optionally reference clusterin level), (b) clusterin level as a % of the reference clusterin level (e.g., 70% of reference clusterin level), (c) decrease (or increase) of clusterin level as compared to reference clusterin level, where the decrease (or increase) can be a % decrease (e.g., 30% decrease (or −30% or 2% increase (or +2%)) or absolute number decrease (e.g., 0.3 mg/mldecrease), or a fold decrease, (e.g., 1.5-fold or 2-fold decrease), (d) a positive signal to indicate cardiac amyloid deposits (e.g., a * or other highlight or + mark) or (e) a negative signal to indicate absence of cardiac amyloid deposits (e.g., absence of mark or other highlight (distinct from the positive indicator) or − mark).

In one embodiment the reference clusterin level data stored in the storage device 30 to be read by the comparison module 40 is the clusterin protein level obtained from a control biological sample of the same type as the biological sample to be tested. Alternatively, the reference clusterin level data is a database, e.g., clusterin protein level in normal pathology unaffected subjects, as disclosed herein. In one embodiment the reference clusterin level data is a clusterin level which is indicative of a subject not having cardiac amyloid deposits, as disclosed herein. In one embodiment the reference clusterin level data is clusterin level obtained from the same type of biological sample from the same subject at an earlier or prior timepoint. In some embodiments, the reference clusterin level data is a clusterin level of the same type of biological sample from a plurality of subjects of the same or a similar demographic background (i.e. age, weight, ethnicity, gender, socio-economic situation and the like), who do not have cardiac amyloid deposits, or do not have an amyloid disease or disorder.

In one embodiment, the reference clusterin data are electronically or digitally recorded and annotated from databases including, but not limited to protein expression databases commonly known in the art, such as Yale Protein Expression Database (YPED), as well as GenBank (NCBI) protein and DNA databases such as genome, ESTs, SNPS, Traces, Celara, Ventor Reads, Watson reads, HGTS, and the like; Swiss Institute of Bioinformatics databases, such as ENZYME, PROSITE, SWISS-2DPAGE, Swiss-Prot and TrEMBL databases; the Melanie software package or the ExPASy WWW server, and the like; the SWISS-MODEL, Swiss-Shop and other network-based computational tools; the Comprehensive Microbial Resource database (available from The Institute of Genomic Research). The resulting reference clusterin level data can be stored in a relational database that may be employed to determine differences in clusterin levels between the reference data or within and among genomes and different populations of individuals.

The “comparison module” 40 can use a variety of available software programs and formats comprising the instructions 70 to compare clusterin level information determined in the determination module to reference clusterin level data. In one embodiment, the comparison module 40 can be configured to use pattern recognition techniques to compare clusterin level information from one or more entries to one or more reference data patterns. The comparison module 40 may be configured using existing commercially-available or freely-available software for comparing protein expression patterns, and may be optimized for particular data comparisons that are conducted.

The comparison module 40, or any other module of the invention such as the output device 50, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. A configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

In one embodiment, the comparison module 40 performs comparisons with mass-spectrometry spectra, for example comparisons of peptide fragment sequence information can be carried out using spectra processed in MATLB with script called “Qcealign” (see for example WO2007/022248, herein incorporated by reference) and “Qpeaks” (Spectrum Square Associates, Ithaca, N.Y.), or Ciphergen Peaks 2.1™ software. The processed spectra can then be aligned using alignment algorithms that align sample data to the control data using minimum entropy algorithm by taking baseline corrected data (see for example WIPO Publication WO2007/022248, herein incorporated by reference). The comparison result can be further processed by calculating ratios. Clusterin proteon level profiles can be discerned.

In one embodiment, the comparison module 40 compares protein expression profiles. Any available comparison software can be used, including but not limited to, the Ciphergen Express (CE) and Biomarker Patterns Software (BPS) package (available from Ciphergen Biosystems, Inc., Freemont, Calif.). Comparative analysis can be done with protein chip system software (e.g., The Proteinchip Suite (available from Bio-Rad Laboratories, Hercules, Calif.). Algorithms for identifying expression profiles can include the use of optimization algorithms such as the mean variance algorithm (e.g. JMP Genomics algorithm available from JMP Software Cary, N.C.).

In one embodiment of the invention, pattern comparison software is used to determine whether the clusterin level data obtained from the biological sample is below a predefined threshold level (i.e. below a threshold level by at least about 30%, or at least about 40% or at least about 50%). In some embodiments, the threshold level is the reference clusterin level data, and in some embodiments the predefined threshold level is about 0.6 mg/ml clusterin protein in a serum biological sample. In some embodiments, where a measured clusterin level is below the predefined threshold level, it is indicative of a subject having, or likely to develop cardiac amyloid deposits and/or amyliodotic cardiomyopathy.

By providing clusterin protein level information in computer-readable form, one can use the clusterin protein level information in readable form in the comparison module 40 to compare with a reference clusterin level data stored on a storage device 30. For example, search programs can be used to identify relevant reference data for comparison with the clusterin level measured by the determination module, for example, to select reference clusterin level data of a subject with the same amyloid disease but without cardiac amyloid deposits and/or amyloidotic cardiomyopathy, or to select reference clusterin data that is specific or matches the particular subject, e.g., reference clusterin data from the same subject (e.g., reference clusterin level data obtained from the same subject, for example at an earlier timepoint, i.e. t₁, t₂, t₃ when comparing against a later timepoint, e.g., t₄ or above). The comparison made in computer-readable form provides computer readable content which can be processed by a variety of means. The content can be retrieved from the comparison module, the retrieved content.

In some embodiments of this aspect and all other aspects of the present invention, the “comparison module” 40 can use a variety of available software programs and formats for the comparison operative to compare protein level information and/or protein sequence information determined in the determination module to reference clusterin protein data. In one embodiment, the comparison module is configured to use pattern recognition techniques to compare sequence information from one or more entries to one or more reference data patterns. The comparison module may be configured using existing commercially-available or freely-available software for comparing patterns, and may be optimized for particular data comparisons that are conducted.

In some embodiments, the comparison module 40 provides computer readable comparison result that can be processed in computer readable form by predefined criteria, or criteria defined by a user, to provide a content (e.g, output data content 80) based on the comparison result that may be stored in the storage device 30, or alternatively, transmitted to an output module 50. In some embodiments, an output device 50 can provide the output information 60 as requested by a user using a display module.

A display module enables display of a content based in part on the comparison result for the user, wherein the displayed content can display output information 60, such as a signal indicative of a subject having or at risk of having cardiac amyloid deposits and/or amyliodotic cardiomyopathy. In some embodiments, a signal can be for example, a display of content indicative of a subject having or at risk of having cardiac amyloid deposits and/or amyliodotic cardiomyopathy on a computer monitor, a printed page of content indicating a subject having or at risk of having cardiac amyloid deposits and/or amyliodotic cardiomyopathy, or a light or sound indicative a subject having or at risk of having cardiac amyloid deposits and/or amyliodotic cardiomyopathy.

In some embodiments, the display module displays just the level of clusterin protein (e.g., clusterin amount in mg/ml). In some embodiments, the display optionally also includes the reference clusterin level (e.g., in mg/ml). Optionally, in some embodiments, the display also places a signal, e.g., a * (asterix) or some other highlighting signal to identify that the measured clusterin level in the biological sample is below the reference clusterin level data. In some embodiments, where the measured clusterin level from the biological sample is significantly below the reference clusterin level data, the display module places a different signal to identify that the measured clusterin level in the biological sample is below by a statistically significant amount the reference clusterin level data. In some embodiments, the p-value is also indicated.

In some embodiments, the output module 50 is a display module, which can transmit the information to an end user, e.g., via a network e.g., world wide web, or produce a report, e.g., printed report, where the output information 60 displayed to the user can include one or more of the following information: (a) absolute clusterin levels (e.g., mg/ml clusterin levels in the biological sample, and optionally reference clusterin level), (b) clusterin level as a % of the reference clusterin level (e.g., 70% of reference clusterin level), (c) decrease (or increase) of clusterin level as compared to reference clusterin level, where the decrease (or increase) can be a % decrease (e.g., 30% decrease (or −30% or 2% increase (or +2%)) or absolute number decrease (e.g., 0.3 mg/mldecrease), or a fold decrease, (e.g., 1.5-fold or 2-fold decrease), (d) a positive signal to indicate cardiac amyloid deposits (e.g., a * or other highlight or + mark) or (e) a negative signal to indicate absence of cardiac amyloid deposits (e.g., absence of mark or other highlight (distinct from the positive indicator) or − mark).

In some embodiments, the output module can transmit information to the user in the form of a scale, for example, a scale of the severity, or amount cardiac amyloid deposits in a subject, or alternatively a scale of a level of low clusterin protein. For example, a scale can be any arbitrary range, for example, numerical scale such as a 1-10 point scale, or a 1-5 point scale, or a 1-4 point scale or a 1-3 point scale. In some embodiments where a numerical scale is used, a lower number indicates a small difference (e.g., less discrepancy) of measured clusterin level as compared to the reference clusterin level, and a higher number indicates a large difference (e.g., large discrepancy) of measured clusterin level as compared to the reference clusterin level. There is no limit to the number range in a scale, and in some embodiments, the scale can be reversed, where the higher number indicates a small difference (e.g., less discrepancy) of measured clusterin level as compared to the reference clusterin level, and the lower number indicates a large difference (e.g., large discrepancy) of measured clusterin level as compared to the reference clusterin level.

In some embodiments, the scale can be similar to the grading system of cancers, where a grade 1 indicates low level of cardiac amyloid deposits, and a grade 5 is indicative of high levels of cardiac amyloid deposits. In some embodiments, the scale can be based on a % decrease of clusterin as compared to a reference clusterin level. For example, a 0 can indicate normal clusterin levels (e.g., no statistical significant difference in the measured clusterin level as compared to the reference clusterin level), and 1, 2, 3, and 4 on a 4-point scale can represent the following: 1 can indicate a 10-25% decrease, 2 can indicate a 26-40% decrease, 3 can indicate a 41-50% decrease and 4 can indicate a 50-100% decrease in clusterin levels as compared to the reference clusterin level used for comparison. Alternatively, an exemplary 4-point scale can also be based absolute clusterin levels, for example, where 0 indicates normal levels (e.g., no statistically significant difference in clusterin levels as compared to about 0.65 mg/ml or other reference clusterin level), and 1, 2, 3, and 4 can represent the following: 1 represents a 0.6-0.4 mg/ml, 2 represents a 0.399-0.3 mg/ml, 3 represents a 0.299-0.2 mg and 4 represents a clusterin level below 0.2 mg/ml.

In some embodiments a scale can be non-numerical, for example for a three point scale, such a non-numerical scale can comprise “mild, moderate or severe”, or alternatively, comprise “low, medium or high” to indicate the level of risk of the subject having cardiac amyloid deposit. Such a non-numerical scale can optionally indicate “normal” where the measured clusterin level is not statistically significant as compared to the reference clusterin level. There is no limit to the number of distinct indicators of such as non-numerical scale.

In some embodiments, a scale (numerical or non-numerical) can be configured to be a comparison to self (e.g., where the reference value is from an earlier timepoint from the same subject), or can be configured to be a comparison based on specific amyloid disease (e.g., a scale specific to TTR-associated diseases or AL-associated diseases), as well as specific types of TTR-diseases, e.g., ATTR, FAP, SSA etc, as well as configured to be a specific comparison to different patient cohorts, for example, to specific patient populations, genotype, age, and gender.

In some embodiments, where the clusterin protein level measured in a biological sample is compared to a reference clusterin level data from the same subject taken at an earlier timepoint, the content can be a signal indicative that the subject from which the biological sample was obtained has, has an increase in clusterin levels, or a decrease in the cardiac amyloid deposits and/or amyliodotic cardiomyopathy.

In one embodiment of the invention, the content based on the comparison result is displayed a on a computer monitor. In one embodiment of the invention, the content based on the comparison result is displayed through printable media. In one embodiment of the invention, the content based on the comparison result is displayed as an indicator light or sound. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for output module and to display of the output data content and displayed information 60 based on the comparison result. It should be understood that other modules as disclosed herein can also be adapted to have a web browser interface. A user interface useful in various embodiments includes, for example, a display screen or a printer or other means for providing a readout of the result of a computer-mediated process. A user interface can also include, for example, an address in a network or on the world wide web to which the results of a process are transmitted and made accessible to one or more users. For example, the user interface can include a graphical user interface comprising an access element that permits entry of data regarding clusterin protein level in a biological sample, as well as an access element that provides a graphical read out of the results of a comparison transmitted to or made available by a processor following execution of the instructions encoded on a computer-readable medium. Through the Web browser, a user may construct requests for retrieving data from the comparison module. Thus, the user will typically point and click to user interface elements such as buttons, pull down menus, scroll bars and the like conventionally employed in graphical user interfaces. The requests so formulated with the user's Web browser are transmitted to a Web application which formats them to produce a query that can be employed to extract the pertinent information related to the clusterin level information, e.g., the display of clusterin levels, or an indication of a subject having or at risk of having cardiac amyloid deposits (e.g., a positive or negative amyloidotic cardiomyopathy test result); display of level of clusterin protein; display of measured clusterin protein level as compared to reference clusterin levels, display of clusterin level for each biological sample measured from a variety of different biological samples (e.g., taken a different timepoints from the same subject or taken from different tissues from the same subject), or display of clusterin protein levels based thereon. In one embodiment, the reference clusterin protein level information is also displayed.

In one embodiment, the output module 50 displays the comparison result and whether the comparison result is indicative a subject having or at risk of having cardiac amyloid deposits.

The present invention therefore provides for systems 10 (and computer readable medium comprising instructions 70 for causing computer systems) to perform methods for determining the presence of cardiac amyloid deposits and/or amyliodotic cardiomyopathy (i.e. a positive or negative c cardiac amyloid deposits and/or amyliodotic cardiomyopathy).

Systems 10 and computer readable medium comprising instructions 70, are merely an illustrative embodiments of the invention for performing methods of determining the presence of, or risk of a subject developing cardiac amyloid deposits and/or amyliodotic cardiomyopathy. Variations of system and computer readable medium, are possible and are intended to fall within the scope of the invention.

The modules of the system 10 or used in the computer readable medium comprising instructions 70 for comparison of the clusterin levels may assume numerous configurations. For example, function may be provided on a single machine or distributed over multiple machines.

A system 10 for performing the comparison processing of the invention may be a general purpose computer used alone or in connection with a specialized processing computer. Such processing may be performed by a single platform or by a distributed processing platform. In addition, such processing and functionality can be implemented in the form of special purpose hardware or in the form of software being run by a general purpose computer. Any data handled in such processing or created as a result of such processing can be stored in a temporary memory, such as in the RAM of a given computer system or subsystem. In addition, or in the alternative, such data may be stored in longer-term storage devices, for example, magnetic disks, rewritable optical disks and so on.

The system 10 may include an operating system (e.g., UNIX) on which runs a relational database management system, a World Wide Web application, and a World Wide Web server. The software on the computer system may assume numerous configurations. For example, it may be provided on a single machine or distributed over multiple machines.

A World Wide Web browser may be used for providing a user interface. Through the Web browser, a user may construct search requests for retrieving data from a sequence database and/or a genomic database. Thus, the user will typically point and click to user interface elements such as buttons, pull down menus, scroll bars, etc. conventionally employed in graphical user interfaces. The requests so formulated with the user's Web browser are transmitted to a Web application which formats them to produce a query that can be employed to extract the pertinent information from relevant databases, e.g. reference level databases. When network employs a World Wide Web server, it supports a TCP/IP protocol. Local networks such as this are sometimes referred to as “Intranets.” An advantage of such Intranets is that they allow easy communication with public domain databases residing on the World Wide Web (e.g., the GenBank World Wide Web site). Thus, in a particular preferred embodiment of the present invention, users can directly access data (via Hypertext links for example) residing on Internet databases using a HTML interface provided by Web browsers and Web servers.

Typically the methods, compositions, computer systems 10 and computable readable media comprising instructions 70 as disclosed herein are useful in the diagnosis and/or determining cardiac amyloid deposits and/or amyliodotic cardiomyopathy in a subject, such as a mammalian subject including a human subject, however, the methods and compositions are equally applicable to non-human subjects, including livestock, domestic and companion animals, and other veterinary and wild-life subjects.

In another embodiment, the methods, systems, compositions and computer readable media as disclosed herein are useful for predicting prognosis of amyliodotic cardiomyopathy in the subject, for example, where a subject which has serum clusterin protein levels lower (i.e. at least about 30%, or at least about 35%, 40%, 45%, 50% lower) as compared to a reference clusterin protein level taken from the same subject at an earlier timepoint is identified as likely to have a poorer prognosis as compared to a subject who is identified to have the same or higher clusterin protein level as compared to the reference clusterin protein level taken from the same subject at an earlier timepoint. Alternatively, where a subject which has serum clusterin protein levels higher (i.e. at least about 30%, or at least about 35%, 40%, 45%, 50% lower) as compared to a reference clusterin protein level taken from the same subject at an earlier timepoint is identified as likely to have a better prognosis as compared to a subject who is identified to have the same or higher clusterin protein level as compared to the reference clusterin protein level taken from the same subject at an earlier timepoint.

Treatment of Subjects Identified to have Amyloidotic Cardiomyopathy (CMP) or Subjects with Diseases and Disorders Related to TTR Amyloidosis

One aspect of the present invention relate to methods for the treatment or prevention of TTR amyloidosis in subjects with, or at risk of developing, TTR amyloidosis, e.g. SSA or FAP. In some embodiments, the disease or disorders associated with TTR amyloidosis is familial amyloidosis, e.g. senile systemic amyloidosis (SSA) or Familial amyloid polyneuropathy (FAP).

It is a general discovery of the present invention that TTR amyloidosis can be treated by administering clusterin protein or clusterin agents (e.g. analogues of clusterin activity, such as but not limited to fragments, variants, isoforms, muteins, functional derivatives etc.) as disclosed herein to prevent the aggregation of TTR variants or wild-type TTR and the formation of TTR comprising fibrils in a subject. The examples of TTR amyloidosis which can be treated with the methods and compositions as disclosed herein are discussed below and serve only to exemplify major forms of TTR amyloidosis and to identify subjects amenable to treatment by the methods using the clusterin protein or clusterin agents as disclosed herein and are not in any way intended to limit the invention.

Without wishing to be bound by theory, protein misfolding and aggregation are now recognized as critical processes in the pathogenesis of a wide range of human diseases. In particular, the deposition of aberrantly folded and self-associated proteins as highly organized β-sheet structured fibrils is the hallmark of the amyloidoses. Amyloid fibrils bind Congo red dye, producing a characteristic apple-green birefringence. In vivo, amyloid deposits have been shown to include a variety of accessory proteins and molecules including serum amyloid P component and glycosaminoglycans (GAGs). Previously, the inventors have proteomics to study the complexity of amyloid deposits in fat (Lavatelli F, et al., Amyloidogenic and Associated Proteins in Systemic Amyloidosis Proteome of Adipose Tissue, Mol. Cell Proteomics 2008, 7:1570-1583).

The two most frequent proteins which can misfold, aggregate, and form amyloid deposits in tissues and organs in systemic amyloidosis are transthyretin (TTR) and immunoglobulin light chain (LC). After albumin, these are the most abundant serum proteins, in fact, TTR is also known as prealbumin from its protein electrophoretic pattern. Inherited mutations in TTR that promote dissociation of the normal tetramer lead to familial TTR (ATTR) amyloidosis while wild type TTR can cause senile systemic (SSA) amyloidosis in older individuals. Clonal LC monomers and fragments are components of the fibrils found in AL or primary amyloidosis, which occurs in association with bone marrow plasma cell disorders. ATTR, SSA, and AL amyloidoses are multiorgan diseases but their most pronounced phenotype is a restrictive cardiomyopathy (CMP), which may present clinically as congestive heart failure, arrhythmias and sudden death (Falk R H, et al., Amyloid Heart Disease, Progress in Cardiovascular Diseases 2010, 52:347-361, Shah et al., Amyloidosis and the Heart: A Comprehensive Review, Arch Intern Med. 2006, 166:1805-1813; Sawyer et al., Cardiac Amyloidosis: Shifting Our Impressions to Hopeful, Curr Heart Fail Rep. 2006, 3:64-71; Hassan et al., Amyloid Heart Disease. New Frontiers and Insights in Pathophysiology, Diagnosis, and Managment, Texas Heart Institute Journal 2005, 32:178-184).

It has been suggested that proteins can aggregate and form fibrils under conditions that promote denaturation including heat, low pH, and with agitation (Stefani et al., Protein aggregation and aggregate toxicity: new insights into protein folding, misfolding diseases and biological evolution, J Mol Med. 2003, 81:678-699.) In vivo, the intra- and extracellular maintenance of proper protein folding and the prevention of aggregation is facilitated by chaperones. Clusterin (CLU), also called apolipoprotein J, is a ubiquitous protein that reportedly functions as an extracellular chaperone and may play a role in the pathologic mechanism of amyloid precursor protein misfolding. CLU has a remarkable conformational adaptability that is typical of molecular chaperones and is attributed to three large molten globule domains, three amphipathic regions, and two coiled-coil α-helices. This molecular structure is responsible for the unique high affinity, low specificity binding property of the protein. Bailey et al., Clusterin, a Binding Protein with a Molten Globule-like Region, Biochemistry 2001, 40:11828-11840, Humphreys et al., Custerin Has Chaperone-like Activity Similar to That of Small Heat Shock Proteins, J Biol Chem. 1999, 274:6875-6881, Nuutinen et al., Clusterin: A forgotten player in Alzheimer's disease, Brain Res. Rev. 2009, 61:89-104.

CLU has also been reported to have been identified in cerebrospinal and seminal fluids, breast milk, urine, and plasma. (Aronow et al., Apolipoprotein J expression at fluid-tissue interfaces: potential role in barrier cytoprotection, PNAS 1993, 90:725-729). CLU gene expression occurs in testis, liver, stomach, brain, kidney and heart. (Rosenberg M E, et al., Clusterin: Physiologic and pathophysiologic considerations, Int. J. Biochem. Cell Biol. 1995, 27:633-645). Studies suggest that the regulation of CLU is directly linked to the heat shock response through HSF1-HSF2 heterocomplex binding to the CLU promoter; thus, modulation of CLU transcription occurs in stress- or disease-induced states (Loison F et al., Up-regulation of the clusterin gene after proteotoxic stress: implication of HSF1-HSF2 heterocomplexes, Biochem J. 2006, 395:223-231). CLU overexpression is observed in Alzheimer's disease (AD) where CLU is found complexed to soluble amyloid beta protein (Aβ) and as a component of amyloid plaques. CLU has been linked to cardiovascular diseases, as it is a constituent of human atherosclerotic plaques, is upregulated at mRNA and protein levels in myocarditis models, and is localized to damaged myocardium in myocardial infarction. (Rosenberg et al., Clusterin: Physiologic and pathophysiologic considerations, Int. J. Biochem. Cell Biol. 1995, 27:633-645; Swertfeger et al., Apolipoprotein J/clusterin induction in myocarditis: A localized response gene to myocardial injury. (Am J Pathol. 1996, 148:1971-1983).

Prompted by these observations, we investigated the role of CLU in amyloidosis. We examined the presence of CLU in amyloid deposits from cardiac tissue specimens in cases of SSA, ATTR, and AL amyloidoses and developed an assay to quantify serum concentrations of CLU and correlate them with cardiac amyloid disease.

### Senile Systemic Amyloidosis (SSA)

Senile systemic amyloidosis (SSA) affects around 25% of the population over 80 years of age, and is a sporadic amyloid disease which is due to the deposit of wild-type TTR to form TTR amyloid fibrils.

### Familial Amyloid Polyneuropathy (FAP)

Familial amyloid polyneuropathy (FAP) affects approximately 1 in 100,000 persons [4]. Familial amyloidoses caused by the aggregation of the misfolding of one of over 100 TTR variants. FAP has been associated with familial amyloid cardiomyopathy, and familial central nervous system amyloidosis. FAP typically presents early and severe impairment in the brain and/or in the peripheral tissues, leading to neurodegeneration and/or organ disruption [4]. Among familial TTR amyloidoses, V30M is the most common FAP variant and has intermediate stability, whereas L55P is the most pathogenic and highly unstable FAP mutant [5].

Normally, TTR circulates in the blood as an active “tetramer” made up of four separate copies, or protein subunits, that interact with each other. These tetramers, normally composed of identical protein subunits, come from two different genes. When one of the genes has a heritable defect, hybrid tetramers form that are composed of mutant and normal subunits. The inclusion of mutated subunits makes the tetramer less stable and causes the four subunits to more easily dissociate. Once the subunits are free, they misfold and reassemble into the rod-like amyloid fibrils. The process of fibril formation causes the disease FAP by compromising peripheral nerve and muscle tissue, disrupting their function and leading to numbness, muscle weakness, and, in advanced cases, failure of the autonomic nervous system, including the gastrointestinal tract. The current treatment for FAP is a liver transplant, which replaces the mutant gene with a normal copy.

An analogous disease to FAP is called familial amyloid cardiomyopathy (FAC), which is caused by deposition of a few variants of TTR in the heart, leads to cardiac dysfunction and ultimately congestive heart failure. It is estimated that approximately 4% of African Americans carry the gene mutation for TTR-V122I, an mutation that predisposes them to FAC.

### AL Amyloidosis

AL amyloid deposition is generally associated with almost any dyscrasia of the B lymphocyte lineage, ranging from malignancy of plasma cells (multiple myeloma) to benign monoclonal gammopathy. At times, the presence of amyloid deposits may be a primary indicator of the underlying dyscrasia.

Without being bound by theory, in AL-amyloidosis, fibrils of AL amyloid deposits are composed of monoclonal immunoglobulin light chains or fragments thereof. More specifically, the fragments are a region of the N-terminal region of the light chain (kappa or lambda), or derivatives thereof, and contain all or part of the variable (V_{L}) domain thereof. More specifically, the fragments do not contain a region of the heavy chain of the variable region (V_{H}). Deposits generally occur in the mesenchymal tissues, causing peripheral and autonomic neuropathy, carpal tunnel syndrome, macroglossia, restrictive cardiomyopathy, arthropathy of large joints, immune dyscrasias, multiple myelomas, as well as ocular dyscrasias. However, it should be noted that almost any tissue, particularly visceral organs such as the heart, may be involved.

In light chain amyloidosis (AL-amyloidosis) a monoclonal immunoglobulin light chain forms the amyloid deposits. See Glenner et al., Amyloid Fibril Proteins: Proof of Homology with Immunoglobulin Light Chains by Sequence Analyses, Science 172:1150-1151, 1971. Amyloid fibrils from patients suffering AL-amyloidosis occasionally contain only intact light chains, but more often they are formed by proteolytic fragments of the light chains which contain the VL domain and varying amounts of the constant domain, or by a mixture of fragments and full-length light chains. Not all light chains from plasma cell dyscrasias form protein deposits; some circulate throughout the body at high concentrations and are excreted with the subject's urine without pathological deposition of the protein in vivo. See Solomon, Clinical Implications of Monoclonal Light Chains, Semin. OncoL 13:341-349, 1986; Buxbaum, Mechanisms of Disease: Monoclonal Immunoglobulin Deposition, Amyloidosis, Light Chain Deposition Disease, and Light and Heavy Chain Deposition Disease, Hematol/Oncol. Clinics of North America 6:323-346, 1992; and Eulitz, Amyloid Formation from Immunoglobulin Chains, Biol. Chef Hoppe-Seyler 373:629-633, 1992. Subjects suffering from AL amyloidosis can be recognized from methods known by a physician of ordinary skill, for example, typical symptoms of amyloidosis depend on the organ affected and include a wide range of symptoms, for example but are not limited to at least one of the following or combinations of; swelling of your ankles and legs, weakness, weight loss, shortness of breath, numbness or tingling in your hands or feet, diarrhea, severe fatigue, an enlarged tongue (macroglossia), skin changes, an irregular heartbeat, and difficulty swallowing. In some instances, the subject may not experience any of the symptoms listed but still has amyloidosis. In addition, a number of diagnostic tests are available for identifying subjects at risk of, or having AL amyloidosis which are commonly known by person skilled in the art, and are encompassed for use in the present invention. These include measurement of including blood and urine tests, though blood or urine tests may detect an abnormal protein, which could indicate amyloidosis, the only definitive test for amyloidosis is a tissue biopsy, in which the physical analyses a small sample of tissue. The tissue sample may be taken from one or more parts of the subject's body, for example abdominal fat, bone marrow or rectum, which is then examined under a microscope in a laboratory to check for signs of amyloid. Occasionally, tissue samples may be taken from other parts of your body, such as your liver or kidney, to help diagnose the specific organ affected by amyloidosis.

In some embodiments, the methods and compositions comprising clusterin and clusterin agents as disclosed herein are useful to treat the main forms of amyloidosis and AL amyloidosis, for example primary amyloidosis, secondary amyloidosis and hereditary amyloidosis.

Primary Amyloidosis.

This most common form of amyloidosis primarily affects your heart, kidneys, tongue, nerves and intestines. Primary amyloidosis isn't associated with other diseases except for multiple myeloma, in a minority of cases. The cause of primary amyloidosis is unknown, but doctors do know that the disease begins in your bone marrow. In addition to producing red and white blood cells and platelets, your bone marrow makes antibodies, the proteins that protect you against infection and disease. After antibodies serve their function, your body breaks them down and recycles them. Amyloidosis occurs when cells in the bone marrow produce antibodies that can't be broken down. These antibodies then build up in your bloodstream. Ultimately, they leave your bloodstream and can deposit in your tissues as amyloid, interfering with normal function.

Secondary Amyloidosis.

This form occurs in association with chronic infectious or inflammatory diseases, such as tuberculosis, rheumatoid arthritis or osteomyelitis, a bone infection. It primarily affects your kidneys, spleen, liver and lymph nodes, though other organs may be involved. Treatment of the underlying disease may help stop this form of amyloidosis.

Hereditary Amyloidosis.

As the name implies, this form of amyloidosis is inherited. This type often affects the nerves, heart and kidneys.

### Amyloidogenic Diseases.

In some embodiments, the present invention provides methods and compositions comprising clusterin and clusterin agents can also be used to treat a disease and/or disorder associated with an amyloidogenic disease. Amyloidogenic diseases are diseases are diseases from the secretion of a protein and/or peptide that aggregates and forms a deposit and is characterized by amyloid deposits or fibril formation. The methods of the present invention provide methods to design and use of clusterin protein or clusterin agents for the treatment of such amyloidogenic diseases or amyloid-related diseases. Such amyloidogenic diseases, or amyloid-related diseases include, for example but is not limited to, Alzheimer's disease, Down's syndrome, vascular dementia or cognitive impairment, type II diabetes mellitus, amyloid A (reactive), secondary amyloidosis, familial mediterranean fever, familial nephrology with urtcaria and deafness (Muckle-wells Syndrome), amyloid lambda L-chain or amyloid kappa L-chain (idiopathic, multiple myeloma or macroglobulinemia-associated) A beta 2M (chronic hemodialysis), ATTR (familial amyloid polyneuropathy (Portuguese, Japanese, Swedish), familial amyloid cardiomyopathy (Danish), isolated cardiac amyloid, (systemic senile amyloidosis) AIAPP or amylin insulinoma, atrial naturetic factor (isolated atrial amyloid), procalcitonin (medullary carcinoma of the thyroid), gelsolin (familial amyloidosis (Finnish), cyctatin C (heritiaty cerebral hemorrhage with amyloidosis (Icelandic), AApo-A-I (familial amyloidotic polyneuropathy—Iowa), AApo-A-II (accelerated senescence in mice), fibrinogen-associated amyloid; and Asor or Pr P-27 (scrapie, Creutzfeld jacob disease, Gertsmann-Straussler-Scheinker syndrome, bovine spongiform encephalitis) and person who are homozygous for the apolipoprotein E4 allele.

Methods of Treatment of Subjects Identified with Low Clusterin Levels

Clusterin has been reported to be used for the treatment or prevention of peripheral neurological diseases because clusterin was found to have a beneficial effect in an animal model of peripheral neuropathy (as disclosed in WO 2004/084932 and US Patent Application US2007/0134260, which are incorporated herein in their entirety by reference). As examples for such peripheral neurological diseases, traumatic nerve injury, nerve trauma, peripheral nerve system (PNS) infections, demyelating diseases of the PNS, neuropathies of the PNS, or carpal tunnel syndrome are mentioned. It was also mentioned in WO 02/22635 that the level of clusterin is increased in the hippocampus and frontal cortex of the brains of Alzheimer's disease patients and that clusterin acts to link the progression of this disease to the complement system.

It has been previously reported in International Patent application WO 2004/084932 and US Patent Application US2007/0134260 the use of clusterin for the treatment and/or prevention of peripheral neurological diseases. The US2007/0134260 application discusses clusterin in the treatment for peripheral neurological diseases such as, inflammatory polyneuropathy, and polyneuropathy as a result toxic agents, from acute febrile diseases from a toxin (e.g. in diphtheria), autoimmune reactions (e.g. in Guillain-Barré syndrome), metabolic disorders (e.g. diabetes mellitus), nutritional polyneuropathy and hereditary neuropathies Types I, II and III. However, in contrast to the present invention, the US2007/0134260 application does not teach, discuss or mention the use of clusterin for any amyloid related diseases or disorders, nor does it discuss or mention clusterin for treatment of TTR amyloidosis, or that clusterin decreases fibril formation, e.g. decreases TTR amyloid fibrils, nor does it discuss or mention clusterin used at low pH to decrease fibril formation or to decrease the presence of amyloid fibrils, e.g. TTR amyloid fibrils.

Further, the international patent application, WO2006/089586 discusses use of a compound with clusterin activity for the treatment of diseases associated with the deposition of abnormal proteins, e.g. misfolded keratins and components of the elastic fibers. While the WO2006/089586 application reports that clusterin as been found in association with β-amyloid in Alzheimer's disease, the WO2006/089586 application does not disclose, teach or discuss the association of clusterin with other amyloid diseases, such as TTR amyloidosis, nor that clusterin decreases TTR amyloid fibrils, nor does it discuss or mention clusterin used at low pH to decrease fibril formation or to decrease the presence of TTR amyloid fibrils. Furthermore, U.S. Patent application 2009/0156525 discusses clusterin (referred to as ApoJ in the '525 application) requires its receptor LRP2 to facilitate the clearance of amyloidogenic Aβ peptide from the interstitial fluid (ISF) and cerebrospinal fluid (CSF). Thus, in contract to the present application, the '525 application indicates clusterin can not function on its own to eliminate amyloid fibrils, but rather requires its ligand LRP2. Thus, the '525 application does not disclose, teach or discuss the association of clusterin with amyloid diseases other than Alzheimer's disease, nor with TTR amyloidosis, nor does it discuss that clusterin decreases TTR amyloid fibrils. The '525 application also does not discuss or mention clusterin used at low pH to decrease fibril formation or to decrease the presence of TTR amyloid fibrils.

It has also been reported in US Patent application 2009/021956 reports the use of cells genetically modified ex vivo to express clusterin for transplantation into a subject to inhibit apoptosis and inflammation and the treatment or inhibition of atherosclerotic lesions and to stabilize existing atherosclerotic plaques. However, unlike the present invention, US Patent application 2009/021956 does not teach, or discuss or mention the use of clusterin for the treatment of any amyloid related diseases or disorders, nor does it discuss or mention clusterin for treatment of TTR amyloidosis, or that clusterin decreases fibril formation, e.g. decreases TTR amyloid fibrils, nor does it discuss or mention clusterin used at low pH to decrease fibril formation or to decrease the presence of amyloid fibrils, e.g. TTR amyloid fibrils. Further, US Patent application 2009/021956 does not teach or suggest or mention direct administration of clusterin or a clusterin agent for the treatment of any amyloid related diseases or disorders, e.g. TTR amyloidosis.

Further, U.S. Pat. No. 5,744,368 discusses an in vitro method to detect the ability of TTR to specifically bind to amyloid β protein, e.g. in CSF. However, the U.S. Pat. No. 5,744,368 does not report the association of clusterin with β-amyloid in Alzheimer's disease, nor does it disclose, teach or discuss or suggest the association of clusterin with other amyloid diseases, such as TTR amyloidosis, nor does it discuss anywhere that clusterin decreases TTR amyloid fibrils or that clusterin used at low pH to decrease fibril formation e.g. to decrease the presence of TTR amyloid fibrils. Additionally, unlike the present invention, the '368 patent does not teach or discuss or disclose that clusterin levels in serum, in particular low levels of clusterin in the serum are indicative of a subject with amyloidotic cardiomyopathy.

In direct contract to the present application which discloses administering clusterin or increasing clusterin activity, patent applications WO 02/22635, US 2002/0128220 and WO 2004/018675 only discuss the reduction of clusterin level by antisense modulation for the treatment of hypercholesterolemia, cardiovascular disorders, hyperproliferative disorders, hyperlipidemic disorders, prostate or renal cell cancer, and melanoma.

As used herein, the term “clusterin agents” is used interchangeably herein with “agonists of clusterin activity” and refer to compounds with clusterin activity, and can be any functionally active or activatable molecule with clusterin activity. In some embodiments, exemplary examples of such molecules which are agonists of clusterin activity are described in WO 2004/084932 and US Patent Application US2007/0134260, which are incorporated herein by reference with respect to all forms of clusterin molecules and clusterin activating compounds. In some embodiments, a clusterin agonist can be any physiologically occurring forms of clusterin, such as human clusterin. The sequence of clusterin is known, e.g. from WO 2004/084932 and US Patent Application US2007/0134260 (disclosed therein as ID NO: 1).

According to the present invention, any clusterin derived from animals, especially mammals, such as human, primate, murine, bovine, porcine, feline or ovine clusterin, are suitable molecules with clusterin activity as long as there is sufficient similarity in order to maintain clusterin activity in e.g. humans, and as long as the resulting molecule will not be immunogenic, or have limited immunogenicity in humans. Further compounds with clusterin activity include biologically active muteins and fragments of clusterin, such as the naturally occurring alpha and beta subunit of clusterin, isoforms, fused proteins, functional derivatives, active fractions or fragments, or circularly permutated derivatives, or salts thereof, as disclosed in WO 2004/084932 and US Patent Application US2007/0134260.

Preferably, the clusterin used in the methods and compositions as disclosed herein is selected from a peptide, a polypeptide or a protein selected from the group consisting of: a) A polypeptide comprising SEQ ID NO: 1; b) A polypeptide comprising amino acids (aa) 23 to 449 of SEQ ID NO: 1; c) A polypeptide comprising amino acids 35 to 449 of SEQ ID NO: 1; d) A polypeptide comprising aa 23 to 227 of SEQ ID NO: 1; e) A polypeptide comprising aa 35 to 227 of SEQ ID NO: 1; f) A polypeptide comprising aa 228 to 449 of SEQ ID NO: 1; g) A mutein of any of (a) to (f), wherein the amino acid sequence has at least 40% or 50% or 60% or 70% or 80% or 90% identity to at least one of the sequences in (a) to (f), h) A mutein of any of (a) to (f) which is encoded by a DNA sequence which hybridizes to the complement of the native DNA sequence encoding any of (a) to (f) under moderately stringent conditions or under highly stringent conditions, i) A mutein of any of (a) to (f) wherein any changes in the amino acid sequence are conservative amino acid substitutions to the amino acid sequences in (a) to (f); j) a salt or an isoform, fused protein, functional derivative, active fraction or circularly permutated derivative of any of (a) to (f).

### Clusterin Agents and Clusterin Variants, Fragments and Variants

Active fractions or fragments may comprise any portion or domain of clusterin, such as the alpha chain or the beta chain separated, or linked to each other e.g. via disulfide bridges, directly fused, or fused via an appropriate linker Active fractions also comprise differentially glycosylated or sialylated forms of clusterin.

The person skilled in the art will appreciate that even smaller portions of clusterin or its two subunits may be enough to exert its function, such as an active peptide comprising the essential amino acid residues required for clusterin function. For example, in some embodiments, fragments of clusterin can be used in the methods and compositions as disclosed herein, e.g. fragments of a clusterin protein can be at least 6, 10, 20, 50, 100, 250, 400 amino acids. Exemplary fragments of clusterin protein can include C-terminal truncations, N-terminal truncations, or truncations of both C- and N-terminals (e.g., deletions of 1, 2, 3, 4, 5, 8, 10, 15, 20, 25, 40, 50, 75, 100 or more amino acids deleted from the N-termini, the C-termini, or both).

A person skilled in the art will appreciate that even smaller portions of clusterin or its two subunits may be enough to exert its biological function, such as an active peptide comprising the essential amino acid residues required for clusterin function. For example, in some embodiments, fragments of clusterin can be used in the methods and compositions as disclosed herein, e.g. fragments of clusterin protein can be, for example, any portion of at least 5 consecutive amino acids of SEQ ID NO: 1, or for example, a polypeptide comprising amino acids 35 to 449 of SEQ ID NO: 1, or a polypeptide comprising amino acids 23 to 449 of SEQ ID NO: 1, or a polypeptide comprising amino acids 23 to 227 of SEQ ID NO: 1, or a polypeptide comprising amino acids 35 to 227 of SEQ ID NO: 1, or a polypeptide comprising amino acids 228 to 449 of SEQ ID NO: 1, or any fragment or portion of those fragments, which have further C-terminal truncations, or N-terminal truncations, or truncations of both C- and N-terminals (e.g., deletions of 1, 2, 3, 4, 5, 8, 10, 15, 20, 25, 40, 50, 75, 100 or more amino acids deleted from the N-termini, the C-termini, or both).

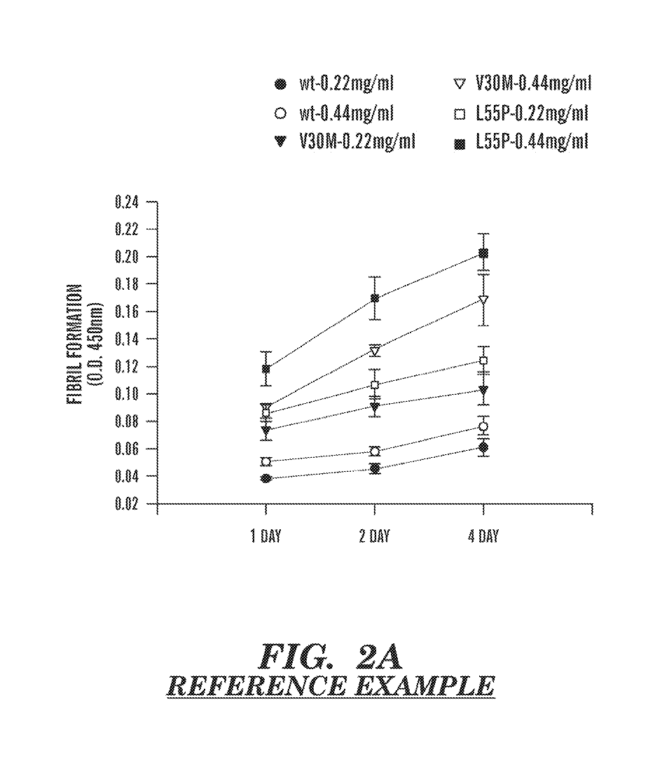

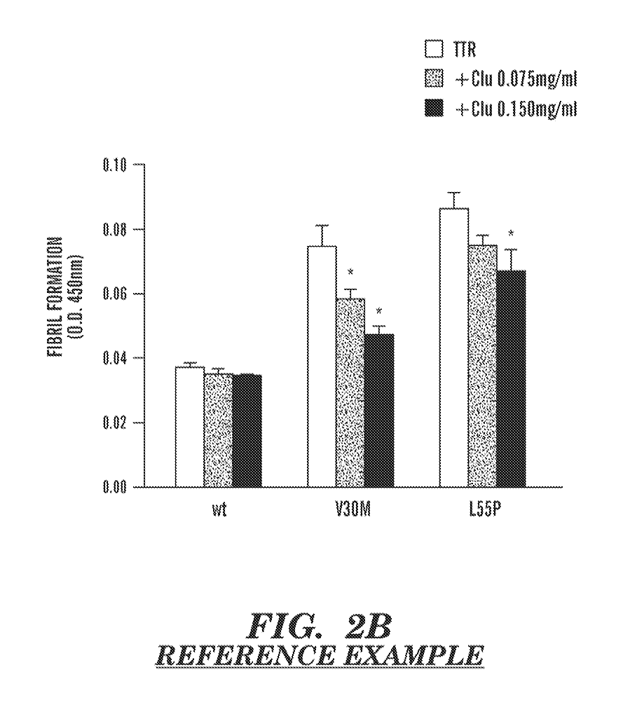

One of ordinary skill in the art will further appreciate that muteins, salts, isoforms, fused proteins, functional derivatives of clusterin, active fractions or circularly permutated derivatives of clusterin, will retain a similar, or even better, biological activity of clusterin. The biological activity of clusterin and muteins, isoforms, fused proteins or functional derivatives, active fractions or fragments, circularly permutated derivatives, or salts thereof, may be measured in the in vitro TTR fibril assay as disclosed herein in the Examples and in FIG. 2. An agent which “retains the biological activity of clusterin” also refer to fragments or portions of clusterin protein thereof that have one or more anti-fibril formation activities, therapeutic or preventive activities of full clusterin protein, as well as to mutants or derivatives of the clusterin polypeptide that retain one or more such activities. By “retain . . . such activity” is meant that a variant or derivative or fragment has at least 50% of the subject activity relative to full length, wild-type clusterin polypeptide, preferably at least 60%, 70%, 80%, 90%, 95%, 96%, 97%, 98%, 99% or 100%, or even greater than 100% of an activity relative to the full length wild-type polypeptide, as for example, as determined using the in vitro fibril formation assay as disclosed in the Examples.

In some embodiment, an active fractions or active fragment of clusterin has an activity which is equal to or better than the activity of full-length clusterin, or which have further advantages, such as a better stability or a lower toxicity or immunogenicity, or they are easier to produce in large quantities, or easier to purify. The person skilled in the art will appreciate that muteins, active fragments and functional derivatives can be generated by cloning the corresponding cDNA in appropriate plasmids and testing them in assay for TTR amyloid fibril formation, as discussed in the Examples section, e.g. as shown in FIG. 2.

The proteins according to the present invention may be glycosylated or non-glycosylated; they may be derived from natural sources, such as body fluids, or it may preferable to produce them recombinantly. Recombinant expression may be carried out in prokaryotic expression systems such as E. coli, or in eukaryotic, such as insect cells, and preferably in mammalian expression systems, such as CHO cells or HEK cells.

As used herein the term “muteins” refers to analogs of clusterin, in which one or more of the amino acid residues of a natural clusterin are replaced by different amino acid residues, or are deleted, or one or more amino acid residues are added to the natural sequence of clusterin, without changing considerably the activity of the resulting products as compared with the wild-type clusterin. These muteins are prepared by known synthesis and/or by site-directed mutagenesis techniques, or any other known technique suitable therefore. Muteins of clusterin. which can be used in accordance with the present invention, or nucleic acid coding thereof, include a finite set of substantially corresponding sequences as substitution peptides or polynucleotides which can be routinely obtained by one of ordinary skill in the art, without undue experimentation, based on the teachings and guidance presented herein. Muteins in accordance with the present invention include proteins encoded by a nucleic acid, such as DNA or RNA, which hybridizes to DNA or RNA and encodes clusterin, in accordance with the present invention, under moderately or highly stringent conditions.

The term “mutant” refers to any change in the genetic material of an organism, in particular a change (i.e., deletion, substitution, addition, or alteration) in a wild-type polynucleotide sequence or any change in a wild-type protein sequence. The term “variant” is used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent).

The term “stringent conditions” refers to hybridization and subsequent washing conditions, which those of ordinary skill in the art conventionally refer to as “stringent”. See Ausubel et al., Current Protocols in Molecular Biology, supra, Interscience, N.Y. 6.3 and 6.4 (1987, 1992), and Sambrook et al. (Sambrook, J. C, Fritsch, E. F., and Maniatis, T. (1989) Molecular Cloning: A Laboratory Manual, Cold Spring Harbor Laboratory Press, Cold Spring Harbor, N.Y.). Without limitation, examples of stringent conditions include washing conditions 12-20° C. below the calculated Tin of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If nixed probes are used, it is preferable to use tetramethyl ammonium chloride (TMAC) instead of SSC (see Ausubel, supra).

As used herein, “variant” with reference to a polynucleotide or polypeptide, refers to a polynucleotide or polypeptide that may vary in primary, secondary, or tertiary structure, as compared to a reference polynucleotide or polypeptide, respectively (e.g., as compared to a wild-type polynucleotide or polypeptide). For example, the amino acid or nucleic acid sequence may contain a mutation or modification that differs from a reference amino acid or nucleic acid sequence. In some embodiments, an peptide immunogen polynucleotide variant may be a different isoform or polymorphism. Peptide immunogen variants can be naturally-occurring, synthetic, recombinant, or chemically modified polynucleotides or polypeptides isolated or generated using methods well known in the art. Changes in the polynucleotide sequence of the variant may be silent. That is, they may not alter the amino acids encoded by the polynucleotide or polypeptide. Where alterations are limited to silent changes of this type, a variant will encode a polypeptide with the same amino acid sequence as the reference. Alternatively, such changes in the polynucleotide sequence of the variant may alter the amino acid sequence of a polypeptide encoded by the reference polynucleotide, resulting in conservative or non-conservative amino acid changes, as described below. Such polynucleotide changes may result in amino acid substitutions, additions, deletions, fusions and truncations in the polypeptide encoded by the reference sequence. Various; codon substitutions, such as the silent changes that produce various restriction sites, may be introduced to optimize cloning into a plasmid or viral vector or expression in a particular prokaryotic or eukaryotic system. A “variant” of clusterin, for example a variant of SEQ ID NO:1 is meant to refer to a molecule substantially similar in structure and function to either the entire molecule, or to a fragment thereof, and includes any agent which retains the biological activity of clusterin at least 50% of the subject activity relative to full length, wild-type clusterin polypeptide, preferably at least 60%, 70%, 80%, 90%, 95%, 96%, 97%, 98%, 99% or 100%, or even greater than 100% of an activity relative to the full length wild-type polypeptide, as for example, as determined using the in vitro fibril formation assay as disclosed in the Examples.

A molecule is said to be “substantially similar” to another molecule if both molecules have substantially similar structures or if both molecules possess a similar biological activity. Thus, provided that two molecules possess a similar activity, they are considered variants as that term is used herein even if the structure of one of the molecules not found in the other, or if the sequence of amino acid residues is not identical. The term “substantial similarity” or “substantially similar” as used in the context of polypeptide sequences, indicates that the polypeptide comprises a sequence with at least 60% sequence identity to a reference sequence, or 70%, or 80%, or 85% sequence identity to the reference sequence, or most preferably 90% identity over a comparison window of about 10-20 amino acid residues. In the context of amino acid sequences, “substantial similarity” further includes conservative substitutions of amino acids. Thus, a polypeptide is substantially similar to a second polypeptide, for example, where the two peptides differ by one or more conservative substitutions.

In a preferred embodiment, any such mutein has at least 40% identity or homology with the sequence of SEQ ID NO: 1 of the annexed sequence listing. More preferably, it has at least 50%, at least 60%, at least 70%, at least 80% or, most preferably, at least 90% identity or homology thereto. Identity reflects a relationship between two or more polypeptide sequences or two or more polynucleotide sequences, determined by comparing the sequences.

In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A %, identity may be determined over the whole length of each of the sequences being compared (so-called global alignment), that is particularly suitable for sequences of the same or very similar length, or over shorter, defined lengths (so-called local alignment), that is more suitable for sequences of unequal length.

As used herein, the term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity.

The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence. The term “similarity”, when used to describe a polypeptide, is determined by comparing the amino acid sequence and the conserved amino acid substitutes of one polypeptide to the sequence of a second polypeptide.

Determination of homologs of the genes or peptides of the present invention may be easily ascertained by the skilled artisan. The terms “homology” or “identity” or “similarity” are used interchangeably herein and refers to sequence similarity between two peptides or between two nucleic acid molecules. Homology and identity can each be determined by comparing a position in each sequence which can be aligned for purposes of comparison. For example, it is based upon using a standard homology software in the default position, such as BLAST, version 2.2.14. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences, respectfully. A sequence which is “unrelated” or “non-homologous” shares less than 40% identity, though preferably less than 25% identity with a sequence of the present application.

In one embodiment, the term “clusterin protein homolog” to a protein identified as associated with the reference clusterin protein amino acid sequence refers to an amino acid sequence that has 40% homology to the full length amino acid sequence of the protein identified as associated with the reference clusterin protein, for example but not limited to at least 40% homology corresponding to SEQ ID NO: 1 of the present invention, or at least about 50%, still more preferably, at least about 60% homology, still more preferably, at least about 70% homology, even more preferably, at least about 75% homology, yet more preferably, at least about 80% homology, even more preferably at least about 85% homology, still more preferably, at least about 90% homology, and more preferably, at least about 95% homology. As discussed above, the homology is at least about 50% to 100% and all intervals in between (i.e., 55%, 60%, 70%, 75%, 80%, 85%, 90%, 95%, 98%, etc.). Also included in the term clusterin protein homologue are clusterin protein variants which comprise a clusterin protein fragment and additional amino acids added to one or both C- or N-terminal ends of the clusterin protein fragment.

The term “substitution” when referring to a clusterin protein, refers to a change in an amino acid for a different entity, for example another amino acid or amino-acid moiety. Substitutions can be conservative or non-conservative substitutions.

Preferred changes for muteins in accordance with the present invention are what are known as “conservative” substitutions. Conservative amino acid substitutions of clusterin polypeptides, may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e, g. fewer than thirty, and preferably under ten, and do not remove or displace amino acids which are critical to a functional conformation, e.g. cysteine residues. Proteins and muteins produced by such deletions and/or insertions come within the purview of the present invention.

The term “conservative substitution,” when describing an clusterin protein, refers to a change in the amino acid composition of the polypeptide that does not substantially alter the polypeptide's activity, fore examples, a conservative substitution refers to substituting an amino acid residue for a different amino acid residue that has similar chemical properties. Conservative amino acid substitutions include replacement of a leucine with an isoleucine or valine, an aspartate with a glutamate, or a threonine with a serine. “Conservative amino acid substitutions” result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not substantially alter activity. Conservative substitution tables providing functionally similar amino acids are well known in the art. For example, the synonymous amino acid groups are the following (amino acid synonymous group in brackets): Ser (Ser, Thr, Gly, Asn), Arg (Arg, Gln, Lys, Glu, His), Leu (Iie, Phe, Tyr, Met, Val, Leu), Pro (Gly, Ala, Thr, Pro), Thr (Pro, Ser, Ala, Gly, His, Gln, Thr), Ala (Gly, Thr, Pro, Ala), Val (Met, Tyr, Phe, Iie, Leu, Val), Gly (Ala, Thr, Pro, Ser, Gly), Iie (Met, Tyr, Phe, Val, Leu, Iie), Phe (Trp, Met, Tyr, Iie, Val, Leu, Phe), Tyr (Trp, Met, Phe, Iie, Val, Leu, Tyr), Cys (Ser, Thr, Cys), His (Glu, Lys, Gln, Thr, Arg, His), Gln (Glu, Lys, Asn, His, Thr, Arg, Gln), Asn (Gln, Asp, Ser, Asn), Lys (Glu, Gln, His, Arg, Lys), Asp (Glu, Asn, Asp), Glu (Asp, Lys, Asn, Gln, His, Arg, Glu), Met (Phe, Iie, Val, Leu, Met) and Trp (Trp). More preferably, the synonymous amino acid groups are the following: Ser (Ser), Arg (Arg, Lys, His), Leu (He, Phe, Met, Leu), Pro (Ala, Pro), Thr (Thr), Ala (Pro, Ala), Val (Met, He, Val), Gly (Gly), He (Met, Phe, Val, Leu, Iie), Phe (Met, He, Tyr, Leu, Phe), Tyr (Phe, Tyr), Cys (Ser, Cys), His (Gln, Arg, His), Gln (Glu, His, Gln), Asn (Asp, Asn), Lys (Arg, Lys), Asp (Asn, Asp), Glu (Gln, Glu), Met (Phe, He, Leu, Met) and Trp (Trp), in some embodiments, the synonymous amino acid groups are the following: Ser (Ser), Arg (Arg), Leu (He, Met, Leu), Pro (Pro), Thr (Thr), Ala (Ala), Val (Val), Gly (Gly), He (Met, Leu, He), Phe (Phe), Tyr (Tyr), Cys (Ser, Cys), His (His), Gln (Gln), Asn (Asn), Lys (Lys), Asp (Asp), Glu (Glu), Met (He, Leu, Met) and Trp (Trp).

Examples of production of amino acid substitutions in proteins which can be used for obtaining muteins of clusterin, polypeptides or proteins, for use in the present invention include any known method steps, such as presented in U.S. Pat. Nos. 4,959,314, 4,588,585 and 4,737,462, to Mark et al.; 5,116,943 to Koths et al., 4,965,195 to Namen et al.; 4,879,111 to Chong et al., and 5,017,691 to Lee et al.); And lysine substituted proteins presented in U.S. Pat. No. 4,904,584 (Shaw et al.).

In addition, individual substitutions, deletions or additions that alter, add or delete a single amino acid or a small percentage of amino acids in an encoded sequence are also “conservative substitutions.” Insertions or deletions are typically in the range of about 1 to 5 amino acids.

As used herein, the term “non-conservative” refers to substituting an amino acid residue for a different amino acid residue that has different chemical properties. The nonconservative substitutions include, but are not limited to aspartic acid (D) being replaced with glycine (G); asparagine (N) being replaced with Iysine (K); or alanine (A) being replaced with arginine (R).

For purposes of classifying amino acids substitutions as conservative or nonconservative, amino acids are grouped as follows: Group I (hydrophobic sidechains): norleucine, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

As used herein, “insertions” or “deletions” are typically in the range of about 1 to 5 amino acids. The variation allowed may be experimentally determined by producing the peptide synthetically while systematically making insertions, deletions, or substitutions of nucleotides in the sequence using recombinant DNA techniques.

As used herein, a “fragment” of a peptide or molecule as used herein refers to any contiguous polypeptide subset of the molecule, that is a peptide that comprises a subset of amino acids in the initial peptide molecule. Fragments of a clusterin protein, for example a fragment of SEQ ID NO:1 is meant to refer to a peptide that is at least one peptide shorter than SEQ ID NO:1 which has the activity as SEQ ID NO:1 are also encompassed for use in the present invention. A fragment of a clusterin protein as disclosed herein, for example a functional fragment of SEQ ID NO: 1 useful in the methods and compositions as disclosed herein have at least 30% of agonist activity as that of SEQ ID NO: 1. Stated another way, a fragment of a clusterin protein is a fragment of SEQ ID NO: 1 which can result in at least 30% of the same activity as compared to SEQ ID NO: 1 to reduce TTR amyloid fibrils in an in vitro or in vivo assay, as disclosed in the Examples 1 and FIG. 2). A fragment of clusterin can also include fragments that have a decreased activity as compared to the wild type clusterin activity by at least 30%. Fragments as used herein are soluble (i.e. not membrane bound). A “fragment” can be at least about 5, at least about 6, least about 7, at least about 8, at least about 9, at least about 10, at least about 11 amino acids, at least about . . . 15 amino acids or more and all integers in between 5 and 15 amino acids. Exemplary fragments include C-terminal truncations, N-terminal truncations, or truncations of both C- and N-terminals (e.g., deletions of, for example, at least 1, at least 2, at least 3, at least 4, at least 5, at least 6, at least 7, at least 8, at least 9, at least 10, at least 15 or more amino acids deleted from the N-termini, the C-termini, or both). One of ordinary skill in the art can create such fragments by simple deletion analysis. Such a fragment of SEQ ID NO: 1 can be, for example, 1, 2, 3, 4, 5, 6, 7, 8, 9 or 10 amino acids or more than 10 amino acids deleted from the N-terminal and/or C-terminal of SEQ ID NO: 1. Persons of ordinary skill in the art can easily identify the minimal peptide fragment of SEQ ID NO: 1 useful in the methods and compositions as disclosed herein, by sequentially deleting N- and/or C-terminal amino acids from SEQ ID NO: 1 and assessing the function of the resulting peptide immunogen fragment. One can create functional fragments with multiple smaller fragments. These can be attached by bridging peptide linkers. One can readily select linkers to maintain wild type conformation.

The term “functional derivative” or “mimetic” are used interchangeably, and refers to a compound which possess a biological activity (either functional or structural) that is substantially similar to a biological activity of the entity or molecule its is a functional derivative of. The term functional derivative is intended to include the muteins, fragments, variants, analogues, fused proteins or chemical derivatives of a molecule, such as functional fragments, functional variants, functional analogues or functional chemical derivative of the peptide immunogens as disclosed herein, for example a protein of SEQ ID NO: 1.

The term “functional derivatives” as used herein, cover derivatives of clusterin, and their muteins and fused proteins, which may be prepared from the functional groups which occur as side chains on the residues or the N- or C-terminal groups, by means known in the art, and are included in the invention as long as they remain pharmaceutically acceptable, i.e. they do not destroy the activity of the protein which is substantially similar to the activity of clusterin, and do not confer toxic properties on compositions containing it.

The term “fused protein” refers to a polypeptide comprising clusterin, or a mutein or fragment thereof, fused with another protein, which e.g. has an extended residence time in body fluids. Clusterin may thus be fused to another protein, polypeptide or the like, e.g. an immunoglobulin or a fragment thereof. Immunoglobulin Fc portions are particularly suitable for production of di- or mulitmeric Ig fusion proteins. The alpha- and beta-chain of clusterin may e.g. be linked to portions of an immunoglobulin in such a way as to produce the alpha- and beta-chain of clusterin dimerized by the Ig Fc portion.

In some embodiments, clusterin derivatives for use in the methods and compositions as disclosed herein can, for example, include polyethylene glycol side-chains, which may mask antigenic sites and extend the residence of clusterin in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanol or carbocyclic aroyl groups) or O-acyl derivatives of free hydroxyl groups (for example that of seryl or threonyl residues) formed with acyl moieties.

As “active fractions” of clusterin, muteins and fused proteins, the present invention covers any fragment or precursors of the polypeptide chain of the protein molecule alone or together with associated molecules or residues linked thereto, e.g. sugar or phosphate residues, or aggregates of the protein molecule or the sugar residues by themselves, provided said fraction has substantially similar activity to clusterin.

The term “salts” herein refers to both salts of carboxyl groups and to acid addition salts of amino groups of clusterin molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of clusterin relevant to the present invention.

Functional derivatives of clusterin may be conjugated to polymers in order to improve the properties of the protein, such as the stability, half-life, bioavailability, tolerance by the human body, or immunogenicity. To achieve this goal, clusterin may be linked e.g. to polyethlyenglycol (PEG). PEGylation may be carried out by known methods, described in WO 92/13095, for example. Therefore, in a preferred embodiment of the present invention, clusterin is PEGylated.

In one embodiment of the invention, a fused clusterin protein can comprise an immunoglobulin (Ig) fusion. The fusion may be direct, or via a short linker peptide which can be as short as 1 to 3 amino acid residues in length or longer, for example, 13 amino acid residues in length. Said linker may be a tripeptide of the sequence E-F-M(Glu-Phe-Met) (SEQ ID NO: 2), for example, —or a 13-amino acid linker sequence comprising Glu-Phe-Gly-Ala-Gly-Leu-Val-Leu-Gly-Gly-Gln-Phe-Met (SEQ ID NO: 3) introduced between clusterin sequence and the immunoglobulin sequence, for instance. The resulting fusion protein has improved properties, such as an extended residence time in body fluids (half-life), an increased specific activity, or increased expression level. The Ig fusion may also facilitate purification of the fused protein.

In a yet another embodiment, clusterin or one or both subunits are fused to the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG2 or IgG4, or other Ig classes, like IgM, for example. Fusion proteins may be monomeric or multimeric, hetero- or homomultimeric. The immunoglobulin portion of the fused protein may be further modified in a way as to not activate complement binding or the complement cascade or bind to Fc-receptors.

In the following description “clusterin” always includes all compounds with clusterin activity, wherever appropriate.

For sequence comparison, for example to determine % identity or homology with a protein sequence of clusterin or a nucleic acid encoding clusterin, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins and Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, such as version 2.2.14 with default parameters for an alignment which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra). One example of algorithm that is suitable for determining percent sequence identify and sequence similarity is the BLAST algorithm, which is described in Altschul et al., J. Mol. Biol. 215:403-410 (1990). Software for performing BLAST analyses is publicly available through the National Center or Biotechnology Information (http://www.ncbi.nlm.nih.gov/). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff& Henikoff, Proc. Natl. Acad. Sci. USA 89, 10915 (1989)).

In some embodiments, clusterin useful in the methods and compositions as disclosed herein is a retro-inverso peptide. A “retro-inverso peptide” refers to a peptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide can contain L-amino acids or D-amino acids, or a mixture of L-amino acids and D-amino acids, up to all of the amino acids being the D-isomer. Partial retro-inverso peptide analogues are polypeptides in which only part of the sequence is reversed and replaced with enantiomeric amino acid residues. Since the retro-inverted portion of such an analogue has reversed amino and carboxyl termini, the amino acid residues flanking the retro-inverted portion are replaced by side-chain-analogous α-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A. and Viscomi, G. C., J. Chem. Soc. Perkin Trans. 1:697-701 (1985); and U.S. Pat. No. 6,261,569, which are incorporated herein in their entirety by reference. Processes for the solid-phase synthesis of partial retro-inverso peptide analogues have been described (EP 97994-B) which is also incorporated herein in its entirety by reference.

One of ordinary skill in the art can easily assess the function of a clusterin protein or fragment or variant thereof to inhibit TTR amyloid formation or decrease the amount of TTR amyloid formation in an assay in vitro (as disclosed in the Example 1 and FIG. 2) as compared to a wild-type clusterin protein corresponding to SEQ ID NO: 1 as disclosed herein. Using such an in vitro assay, if the fragment or variant or derivative of clusterin has at least 30% of the biological activity of the clusterin protein corresponding to SEQ ID NO: 1 as disclosed herein, then the clusterin fragment is considered a valid clusterin fragment and can used in the methods and compositions as disclosed herein. In some embodiments, a fragment of SEQ ID NO: 1 can be less than 9, or less than 8 or less than 7, or less than 6, or less than 5 amino acids of SEQ ID NO: 1. However, as stated above, the fragment must be at least about 5, at least about 6, least about 7, at least about 8, at least about 9, at least about 10, at least about 11 amino acids or more or any integers in between. In some embodiments, a preferred fragment of clusterin is about 5 or 6 amino acids in length. As discussed above, a peptide immunogen variant can comprise a clusterin protein fragment and additional amino acids added to one or both C- or N-terminal ends of the clusterin protein fragment.

An “analog” of a molecule a clusterin protein, for example an analogue of SEQ ID NO: 1 is meant to refer to a molecule substantially similar in function to either the entire molecule or to a fragment thereof. As used herein, a molecule is said to be a “chemical derivative” of another molecule when it contains additional chemical moieties not normally a part of the molecule. Such moieties can improve the molecule's solubility, absorption, biological half life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., MackPubl., Easton, Pa. (1990).

In some embodiments, the clusterin proteins for use in the methods and compositions as disclosed herein are typically substantially pure. This means that a clusterin protein is typically at least about 50% w/w (weight/weight) purity, as well as being substantially free from interfering proteins and contaminants. Sometimes a clusterin protein is at least about 80% w/w and, more preferably at least 90 or about 95% w/w purity. However, using conventional protein purification techniques, homogeneous peptides of at least 99% w/w can be obtained.

In some embodiments, clusterin proteins or clusterin agents as disclosed herein, e.g. clusterin fragments, analogs and variants can be synthesized by solid phase peptide synthesis or recombinant expression, or can be obtained from natural sources according to standard methods well known in the art. Additionally, other compositions, methods of extracting and determining sequences are known in the art available to persons desiring to make and use such compositions. Automatic peptide synthesizers may be used to make such compositions and are commercially available from numerous manufacturers, such as Applied Biosystems (Perkin Elmer; Foster City, Calif.), and procedures for preparing synthetic peptides are known in the art. Recombinant expression can be in bacteria, such as E. coli, yeast, insect cells or mammalian cells; alternatively, proteins can be produced using cell free in vitro translation systems known in the art. Procedures for recombinant expression are described by Sambrook et al., Molecular Cloning: A Laboratory Manual (C.S.H.P. Press, NY 2d ed., 1989).

In some embodiments, the clusterin protein also include longer polypeptides that include, for example, a clusterin protein, active fragment or analog together with other amino acids. Such polypeptides can be screened for prophylactic or therapeutic efficacy in in vitro assays as discussed in the Examples sections, or in animal models of TTR amyloidosis. A clusterin protein or clusterin agent as disclosed herein, e.g. an analog, active fragment or other polypeptide can be administered in associated form or in dissociated form. The clusterin protein or clusterin agent can also include multimers of monomeric of clusterin protein, as well as clusterin protein conjugates, for example clusterin protein conjugated to other antigens, or carrier proteins for decreased immunogenicity of the clusterin protein.

### Delivery Mechanisms

In a further variation, a clusterin protein can be presented as a viral or bacterial vaccine as part of the pharmaceutical composition. A nucleic acid encoding clusterin protein can be incorporated into a genome or episome of the virus or bacteria. Optionally, the nucleic acid is incorporated in such a manner that the clusterin protein is expressed as a secreted protein or as a fusion protein with an outer-surface protein of a virus or a transmembrane protein of a bacteria so that the peptide is displayed. Viruses or bacteria used in such methods should be nonpathogenic or attenuated. Suitable viruses include adenovirus, HSV, Venezuelan equine encephalitis virus and other alpha viruses, vesicular stomatitis virus, and other rhabdo viruses, vaccinia and fowl pox. Suitable bacteria include Salmonella and Shigella. Fusion of a clusterin protein or analogue of clusterin activity to a helper peptide, for example HBsAg of HBV is also encompassed for use in the present invention.

In some embodiments, pharmaceutical compositions comprising clusterin proteins and clusterin agents as disclosed herein can also optionally comprise other peptides and other compounds that do not necessarily have a significant amino acid sequence similarity with clusterin protein but nevertheless serve as mimetics of clusterin protein and reduce the formation of TTR amyloid fibrils, which comprise TTR variants and/or wild type TTR proteins.

### Pharmaceutical Compositions

Another aspect of the present invention further relates to pharmaceutical compositions, particularly useful for prevention and/or treatment of a disease or disorder related to TTR amyloidosis, e.g. SSA or FAP, which comprise a therapeutically effective amount of clusterin.

Preferably, however, the disease or disorder to be treated or prevented by the methods and compositions of present invention is selected from (i) TTR amyloidosis, (ii) senile systemic amyloidosis (SSA), (iii) familial amyloidosis polyneuropathy (FAP) (iv) neurodegenerative disorders, where abnormal proteins such as TTR are disposed in the extracellular space, for example, as a result of misfolded TTR, such as TTR variants V30M and L55P.

Clusterin can preferably be used in an amount of about 0.001 to 10 mg/kg or about 0.01 to 5 mg/kg or body weight or about 0.1 to 3 mg/kg of body weight or about 0.1 to 0.2 mg/kg of body weight or about 1 to 2 mg/kg of body weight. Further preferred amounts of clusterin are amounts of about 0.1 to 1000 μg/kg of body weight or about 1 to 100 μg/kg of body weight or about 10 to 50 μg/kg of body weight. In some embodiments, clusterin can be used at a concentration of about 0.075 mg/ml or 0.150 mg/ml or a higher concentration of 0.150 mg/ml.

In some embodiments, clusterin is used at an effective amount of about 0.15 mg/ml of clusterin to prevent the amyloid fibril formation about 0.22 mg/ml of TTR variants (e.g. V30M, L55P variants). As such, clusterin can be used in doses which range at about a 1:1 ratio of clusterin protein to TTR protein (w/w), or in other embodiments, ratios of about 0.75:1.0 or about 1.0:1.25 or about 1.0:1.3 or about a 1.0:1.5 or about a 1.0:1.75 or about 1.0 to 2.0 of clusterin protein to amyloid protein concentration (e.g. TTR proteins, such as TTR variants V30 and L55P) can be used. In alternative embodiments, ratios of about 0.75:1.0 or about 1.0:1.25 or about 1.0:1.3 or about a 1.0:1.5 or about a 1.0:1.75 or about 1.0 to 2.0 of amyloid protein concentration (e.g. TTR proteins, such as TTR variants V30 and L55P) to clusterin protein can be used.

Preferably, the medicament according to the present invention is administered to the human patient in a dose of 0.001 to 10 mg/kg, preferably from 0.01 to 5 mg/kg, especially 0.1 to 0.2 mg/kg body weight.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, epidural, topical, intrathecal, rectal, and intranasal routes. Any other therapeutical efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the protein (s) according to the invention can be administered together with other components of biologically active agents such as pharmaceutically acceptable surfactants, excipients, carriers, diluents and vehicles.

For parenteral (e.g. intravenous, subcutaneous, intramuscular) administration, the active protein (s) can be formulated as a solution, suspension, emulsion or lyophilized powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The bioavailability of the active protein (s) according to the invention can also be ameliorated by using conjugation procedures which increase the half-life of the molecule in the human body, for example linking the molecule to polyethlyenglycol, as described in WO 92/13095, which is incorporated herein in its entirety by reference.

In some embodiments, a therapeutically effective amount of clusterin protein will be a function of many variables, including the type of protein, the affinity of the protein, any residual cytotoxic activity exhibited by the antagonists, the route of administration, the clinical condition of the patient (including the desirability of maintaining a non-toxic level of endogenous clusterin activity).

In some embodiments, the route of administration is administration by subcutaneous route. Intramuscular administration is another alternative route of administration according to the invention.

In some embodiments, clusterin is administered daily or every other day. The daily doses are usually given in divided doses or in sustained release form effective to obtain the desired results. Second or subsequent administrations can be performed at a dosage which is the same, less than or greater than the initial or previous dose administered to the individual. A second or subsequent administration can be administered during or prior to onset of the disease.

According to the invention, clusterin can be administered pro-phylactically or therapeutically to a subject prior to, simultaneously or sequentially with other therapeutic regimens or agents (e.g. multiple drug regimens), in a therapeutically effective amount.

In some embodiments, clusterin or clusterin agents are administered simultaneously with other therapeutic agents can be administered in the same or different compositions. Compounds promoting or enhancing clusterin activity are e.g, the agonists of clusterin activity disclosed in WO 2004/084932 and US Patent Application US2007/0134260, which are incorporated herein in their entirety by reference, and include molecules stimulating or mimicking clusterin activity, such as agonistic antibodies of a clusterin receptor, small molecular weight agonists activating signalling through a clusterin receptor, enhancers of (endogeneous) clusterin expression. Also gene activation technology can be used to enhance expression of clusterin, ApoA-I, beta-amyloid peptide, complement components, gluthathione-S-transferase, gp330/megalin, heparin, immunoglobulins, lipids, paraoxonase, prion peptide, SIC (streptococcal inhibitor of complement), S. aureus cell surface and TGF-beta receptor are reported clusterin binding ligands and can also be used for triggering clusterin activity TGF-β and IL-1 were shown to induce clusterin expression. Furthermore heat shock factor 1 (HSF1) inducers or activators could be used as inducers of clusterin expression.

Positive effects of administration of clusterin or a compound promoting or enhancing clusterin activity has been demonstrated in treatment of Alzheimer's disease (WO 02/22635) and other diseases associated with deposition of abnormal proteins (See WO2006/089586), which are both incorporated herein in their entirety by reference.

The exact route of administration as well as the optimal dosages can be determined for each specific case, mainly based on the nature of the disease or disorder and on the stage of this disease. Preferably, the medicament according to the present invention is applied locally or systemically, in particular, intravenously, orally, parenterally, epicutaneously, subcutaneously, intrapulmonarily by inhalation or bronchoalveolar lavage, intramuscularily, intracranially, locally into intervertebral discs or other connective tissues.

The present invention provides pharmaceutical compositions for administration to a subject in methods for the treatment or prevention (e.g. prophylactic treatment) for diseases and disorders related with TTR amyloidosis. In particular, according to the invention provided herein, it is possible to prevent progression of, ameliorate the symptoms of, and/or reduce TTR amyloid fibril formation, when an effective amount or effective dose of an pharmaceutical composition comprising a clusterin protein the present invention is administered to the subject in need thereof.

In prophylactic applications, pharmaceutical compositions (or medicants) comprising clusterin and agonists of clusterin activity can be administered to a subject susceptible to, or otherwise at risk of, a particular disease, for example a disease or disorder associated with TTR amyloidosis in an amount sufficient to eliminate or reduce the risk or delay the outset of the disease.

In therapeutic applications, pharmaceutical compositions (or medicants) are administered to a subject suspected of, or already suffering from such a disease in an amount sufficient to cure, or at least partially arrest, the symptoms of the disease and its complications. An amount adequate to accomplish this is defined as a therapeutically- or pharmaceutically-effective dose. In both prophylactic and therapeutic regimes, clusterin proteins and agents functioning as agonists of clusterin activity are usually administered in several dosages until a sufficient immune response has been achieved. Typically, the presence of amyloid formation can be monitored in vitro and in vivo and repeated dosages are given amyloid formation begins to increase.

The present invention provides therapeutic compositions useful for practicing the therapeutic methods described herein. Therapeutic compositions of the present invention can further comprise a physiologically tolerable carrier together with clusterin protein or an agonist of clusterin activity as disclosed herein, or derivatives, fragments or variants thereof or a vector capable of expressing a clusterin protein as disclosed herein, or derivatives, fragments or variants thereof as described herein, dissolved or dispersed therein as an active ingredient.

The preparation of a pharmacological composition that contains active ingredients (e.g. clusterin protein and/or agonists of clusterin activity) dissolved or dispersed therein is well understood in the art and need not be limited based on formulation. Typically such compositions are prepared as injectable either as liquid solutions or suspensions, however, solid forms suitable for solution or suspension in liquid prior to use can also be prepared. The preparation can also be emulsified or presented as a liposome composition. In some embodiments, a clusterin protein and/or agonists of clusterin activity can be mixed with excipients which are pharmaceutically acceptable and compatible with the active ingredient and in amounts suitable for use in the therapeutic methods described herein. In addition, if desired, the composition comprising clusterin protein and/or agonists of clusterin activity can contain minor amounts of auxiliary substances such as wetting or emulsifying agents, pH buffering agents and the like which enhance the effectiveness of the active ingredient.

In some embodiments, the pH buffering agents can be used to maintain the pH of the composition below a pH of 7.6, preferably at a pH less than about pH 6.0, and within a range of pH 6.0 to pH 4.5.

Physiologically tolerable carriers (i.e. physiologically acceptable carriers) are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

For topical application, the carrier may be in the form of, for example, and not by way of limitation, an ointment, cream, gel, paste, foam, aerosol, suppository, pad or gelled stick.

The amount of a clusterin protein and/or agonists of clusterin activity as disclosed herein, or derivatives, fragments or variants thereof that will be effective in the treatment of a particular disorder or condition (e.g. TTR amyloidosis) will depend on the nature of the disorder or condition (e.g. familial TTR amyloidosis such as FAP and FAC, or SSA) and can be determined by standard clinical techniques. In addition, assays such as an in vitro fibril formation assay as discussed herein in the Examples may optionally be employed to help identify optimal dosage ranges.

The precise dose to be employed in the formulation will also depend on the route of administration, and the seriousness of the disease or disorder, and should be decided according to the judgment of the practitioner and each subject's circumstances, and is discussed further below.

The route of administration can be any route known to persons skilled in the art, for example but not limited to parenteral, including intravenous and intraarterial administration, intrathecal administration, intraventricular administration, intraparenchymal, intracranial, intracisternal, intrastriatal, and intranigral administration.

In some embodiments, the present invention also contemplates an article of manufacture which is a labeled container for providing a clusterin protein or an agonist of clusterin activity as disclosed herein, or derivatives, fragments or variants thereof. In some embodiments, an article of manufacture comprises packaging material and a pharmaceutical agent (e.g. a clusterin protein or an agonist of clusterin activity) contained within the packaging material.

In some embodiments, a pharmaceutical agent (e.g. a clusterin protein or an agonist of clusterin activity) in an article of manufacture is any of the compositions of the present invention suitable for providing a clusterin protein or an agonist of clusterin activity as disclosed herein, or derivatives, fragments or variants thereof and formulated into a pharmaceutically acceptable form as described herein according to the disclosed indications. Thus, the composition can comprise a clusterin protein or an agonist of clusterin activity as disclosed herein, or derivatives, fragments or variants thereof or a DNA molecule which is capable of expressing the a clusterin protein or an agonist of clusterin activity as disclosed herein, or derivatives, fragments or variants thereof.

The article of manufacture contains an amount of pharmaceutical agent (e.g. a clusterin protein or an agonist of clusterin activity) sufficient for use in treating a disease or disorder associated with amyloidosis, e.g. TTR amyloidosis as indicated herein, either in unit or multiple dosages. The packaging material comprises a label which indicates the use of the pharmaceutical agent contained therein, e.g., for the treatment of disease or disorder associated with amyloidosis, e.g. TTR amyloidosis, or for other indicated therapeutic or prophylactic uses. The label can further include instructions for use and related information as may be required for marketing. The packaging material can include container(s) for storage of the pharmaceutical agent.

As used herein, the term packaging material refers to a material such as glass, plastic, paper, foil, and the like capable of holding within fixed means a pharmaceutical agent. Thus, for example, the packaging material can be plastic or glass vials, laminated envelopes and the like containers used to contain a pharmaceutical composition including the pharmaceutical agent. In preferred embodiments, the packaging material includes a label that is a tangible expression describing the contents of the article of manufacture and the use of the pharmaceutical agent contained therein.

### Treatment Regimes.

Effective doses of the compositions of the present invention, for the treatment of the above described clinical symptoms, for example disease or disorder associated with amyloidosis, e.g. TTR amyloidosis vary depending upon many different factors, including means of administration, target site, physiological state of the subject, whether the subject is human or an animal, other medications administered, and whether treatment is prophylactic or therapeutic.

The dosage and frequency of administration can vary depending on whether the treatment is prophylactic or therapeutic. In prophylactic applications, a relatively low dosage is administered at relatively infrequent intervals over a long period of time. Some subjects continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In some embodiments, the subject is a human, and in alternative embodiments the subject is a non-human mammal. Treatment dosages need to be titrated to optimize safety and efficacy. The amount of clustering protein or an agonist of clusterin activity depends on the protein forming the amyloid fibrils as well as whether another agent is also administered.

In some embodiment, the amount of a clusterin protein for administration sometimes varies from 1 μg-500 μg per subject and more usually from 5-500 μg per administration for human administration. Occasionally, a higher dose of 0.5-5 mg per injection is used. Typically about 10, 20, 50 or 100 μg is used for administration to a human. Preferably, the medicament according to the present invention is administered to the human patient in a dose of 0.001 to 10 mg/kg, preferably from 0.01 to 5 mg/kg, especially 0.1 to 0.2 mg/kg body weight. Clusterin can preferably be used in an amount of about 0.001 to 10 mg/kg or about 0.01 to 5 mg/kg or body weight or about 0.1 to 3 mg/kg of body weight or about 0.1 to 0.2 mg/kg of body weight or about 1 to 2 mg/kg of body weight. Further preferred amounts of clusterin are amounts of about 0.1 to 1000 μg/kg of body weight or about 1 to 100 μg/kg of body weight or about 10 to 50 μg/kg of body weight. In some embodiments, clusterin can be used at a concentration of about 0.075 mg/ml or 0.150 mg/ml or a higher concentration of 0.150 mg/ml.

The timing of administration can vary significantly from once a day, to once a year, to once a decade. Generally, in accordance with the teachings provided herein, effective dosages can be monitored by obtaining a fluid sample from the subject, generally a blood serum sample, and determining the level of clusterin in the serum sample, using methods well known in the art and readily adaptable to the methods as disclosed herein.

On any given day that a dosage of a clusterin protein or agonist of clusterin protein activity as disclosed herein is given, the dosage is greater than about 1 μg/subject and usually greater than 10 μg/subject if adjuvant is also administered, and greater than 10 μg/subject and usually greater than 100 μg/subject in the absence of adjuvant. Doses for a clusterin protein or agonist of clusterin protein as disclosed herein are determined according to standard dosing and titering methods, taken in conjunction with the teachings provided herein.

The pharmaceutical compositions comprising a clusterin protein or agonist of clusterin protein activity as disclosed herein can be administered by parenteral, topical, intravenous, oral, subcutaneous, intraperitoneal, intranasal or intramuscular means for prophylactic and/or therapeutic treatment. Typical routes of administration of a clusterin protein or agonist of clusterin protein activity as disclosed herein are intramuscular (i.m.), intravenous (i.v.) or subcutaneous (s.c.), although other routes can be equally effective. Intramuscular injection is most typically performed in the arm or leg muscles. In some methods, t a clusterin protein or agonist of clusterin protein activity as disclosed herein or other pharmaceutical compositions are injected directly into a particular tissue where the amyloid fibrils forms or is typically located. In some methods, particular pharmaceutical compositions comprising a clusterin protein or agonist of clusterin protein activity as disclosed herein for the treatment of amyloidogenic diseases of the brain are administered directly to the head or brain via injection directly into the cranium. In some methods, a clusterin protein or agonist of clusterin protein activity as disclosed herein are administered as a sustained release composition or device, such as a Medipad™ device.

In some embodiments, clusterin protein or agonist of clusterin protein activity as disclosed herein can optionally be administered in combination with other agents that are at least partly effective in treatment of amyloidosis associated diseases, for example, SSA, FAP, FAC and AL amyloidosis. In some embodiments, a clusterin protein or agonist of clusterin protein activity as disclosed herein can also be administered in conjunction with other agents that increase passage of the a clusterin protein or agonist of clusterin protein activity as disclosed herein across the blood-brain barrier.

In some embodiments, a clusterin protein or agonist of clusterin protein activity as disclosed herein o are often administered as pharmaceutical compositions comprising an active therapeutic agent, i.e., and a variety of other pharmaceutically acceptable components. See Remington's Pharmaceutical Science (15th ed., Mack Publishing Company, Easton, Pa., 1980). The form of administration depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, non-therapeutic, non-immunogenic stabilizers and the like. However, some reagents suitable for administration to animals may not necessarily be used in compositions for human use.

Pharmaceutical compositions can also optionally comprise include large, slowly metabolized macromolecules such as proteins, polysaccharides, polylactic acids, polyglycolic acids and copolymers (such as latex functionalized sepharose, agarose, cellulose, and the like), polymeric amino acids, amino acid copolymers, and lipid aggregates (such as oil droplets or liposomes).

For parenteral administration, a clusterin protein or agonist of clusterin protein activity as disclosed herein can be administered as injectable dosages of a solution or suspension of the substance in a physiologically acceptable diluent with a pharmaceutical carrier which can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249, 1527 (1990) and Hanes, Advanced Drug Delivery Reviews 28, 97-119 (1997). In some embodiments, a clusterin protein or agonist of clusterin protein activity as disclosed herein can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications. For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins (See Glenn et al., Nature 391, 851 (1998)). Co-administration can be achieved by using the components as a mixture or as linked molecules obtained by chemical crosslinking or expression as a fusion protein.

Alternatively, transdermal delivery can be achieved using a skin path or using transferosomes (Paul et al., Eur. J. Immunol. 25, 3521-24 (1995); Cevc et al., Biochem. Biophys. Acta 1368, 201-15 (1998)).

The variation in primary structure of a clusterin protein or agonist of clusterin protein activity as disclosed herein are useful in the present invention, for instance, may include deletions, additions and substitutions. The substitutions may be conservative or non-conservative. The differences between the natural protein (native) and the variant of a peptide immunogen generally conserve desired properties, mitigate or eliminate undesired properties and add desired or new properties.

In some embodiments, a clusterin protein or agonist of clusterin protein activity as disclosed herein, or variants or fragments or derivatives thereof can also be a fusion polypeptide, fused, for example, to a polypeptide that targets the product to a desired location, or, for example, a tag that facilitates its purification, if so desired. Fusion to a polypeptide sequence that increases the stability of a clusterin protein or agonist of clusterin protein activity as disclosed herein is also contemplated, without substantially interfering with its ability to decrease fibril formation, e.g. TTR amyloid fibril formation in vivo or in vitro is desired. For example, fusion to a serum protein, e.g., serum albumin, can increase the circulating half-life of a clusterin protein. Tags and fusion partners can be designed to be cleavable, if so desired. Another modification specifically contemplated is attachment, e.g., covalent attachment, to a polymer. In one aspect, polymers such as polyethylene glycol (PEG) or methoxypolyethylene glycol (mPEG) can increase the in vivo half-life of proteins to which they are conjugated. Methods of PEGylation of polypeptide agents are well known to those skilled in the art, as are considerations of, for example, how large a PEG polymer to use.

In another aspect, biodegradable or absorbable polymers can provide extended, often localized, release of polypeptide agents, e.g. a clusterin protein or agonist of clusterin protein activity as disclosed herein. The potential benefits of an increased half-life or extended release for a therapeutic agent are clear. A potential benefit of localized release is the ability to achieve much higher localized dosages or concentrations, for greater lengths of time, relative to broader systemic administration, with the potential to also avoid possible undesirable side effects that may occur with systemic administration.

Bioabsorbable polymeric matrix suitable for delivery of a clusterin protein or agonist of clusterin protein activity as disclosed herein, or variants or fragments or derivatives thereof can be selected from a variety of synthetic bioabsorbable polymers, which are described extensively in the literature. Such synthetic bioabsorbable, biocompatible polymers, which may release proteins over several weeks or months can include, for example, poly-hydroxy acids (e.g. polylactides, polyglycolides and their copolymers), polyanhydrides, polyorthoesters, segmented block copolymers of polyethylene glycol and polybutylene terephtalate (Polyactive™), tyrosine derivative polymers or poly(ester-amides). Suitable bioabsorbable polymers to be used in manufacturing of drug delivery materials and implants are discussed e.g. in U.S. Pat. Nos. 4,968,317, 5,618,563, among others, and in “Biomedical Polymers” edited by S. W. Shalaby, Carl Hanser Verlag, Munich, Vienna, New York, 1994 and in many references cited in the above publications. The particular bioabsorbable polymer that should be selected will depend upon the particular patient that is being treated.

### Nucleic Acid Encoding the Clusterin Proteins or Clusterin Agents.

Another aspect of the present invention provides methods for the treatment of a disease or disorder related to TTR amyloidosis, e.g. SSA or FAP by administering a clusterin nucleic acid, e.g. as a naked nucleic acid molecule, e.g. by intramuscular injection.

A clusterin nucleic acid molecule for the treatment of a disease or disorder related to TTR amyloidosis, e.g. SSA or FAP can further comprise vector sequences, such as viral sequence, useful for expression of the gene encoded by the nucleic acid molecule in the human body, preferably in the appropriate cells or tissues. Therefore, in some embodiments, the nucleic acid molecule further comprises an expression vector sequence. Expression vector sequences are well known in the art, they comprise further elements serving for expression of the gene of interest. They may comprise regulatory sequence, such as promoter and enhancer sequences, selection marker sequences, origins of multiplication, and the like. A gene therapeutic approach is thus used for treating and/or preventing a disease related to TTR amyloidosis, e.g. SSA or FAP. Advantageously, the expression of clusterin will then be in situ.

In some embodiments of the invention, an expression vector comprising a clusterin nucleic acid molecule can be administered by intramuscular injection. The use of a vector for inducing and/or enhancing the endogenous production of clusterin in a cell normally silent for expression of clusterin, or which expresses amounts of clusterin which are not sufficient, are also contemplated according to the invention. The vector may comprise regulatory sequences functional in the cells desired to express clusterin. Such regulatory sequences may be promoters or enhancers, for example. The regulatory sequence may then be introduced into the appropriate locus of the genome by homologous recombination, thus operably linking the regulatory sequence with the gene, the expression of which is required to be induced or enhanced. The technology is usually referred to as “endogenous gene activation” (EGA), ands it is described e.g. in WO 91/09955, which is incorporated herein in its entirety by reference.

In some embodiments, the method for treatment of a disease or disorder related to TTR amyloidosis, e.g. SSA or FAP comprises administering a cell which has been genetically modified to express clusterin. In some embodiments, the cell which is modified to express clusterin is modified ex vivo and then administered to a subject for the treatment of a disease or disorder related to TTR amyloidosis, e.g. SSA or FAP. Thus, in some embodiments the present invention relates to the use of a cell that has been genetically modified to produce clusterin for the method and compositions (e.g. in the manufacture of a medicament) for the treatment of a disease or disorder related to TTR amyloidosis, SSA or FAP. Thus, a cell therapeutic approach is also encompassed in the methods as disclosed herein to deliver the clusterin molecule to the appropriate parts of the human body, e.g., for the treatment of a disease or disorder related to TTR amyloidosis, e.g. SSA or FAP

In some embodiments, clusterin used in methods for the treatment of diseases associated with TTR amyloidosis can also be induced by administration of nucleic acids encoding clusterin, or clusterin agents. Such nucleic acids can be DNA or RNA. A nucleic acid segment encoding a clusterin, or a variant, mutein or fragment thereof as disclosed herein is typically linked or operatively linked to regulatory elements, such as a promoter and enhancer that allows for expression of the DNA segment in the intended target cells of a subject. For expression in blood cells, as for the expression of clusterin in the blood, promoters and enhancer elements from light or heavy chain immunoglobulin genes or the CMV major intermediate early promoter and enhancer are suitable to direct expression. The linked regulatory elements and coding sequences are often cloned into a vector. For administration of double-chain antibodies, the two chains can be cloned in the same or separate vectors.

Without limitation and as well-known to those skilled in the art, the invention encompasses the nucleic acid sequences that encode clusterin proteins or variants, fragments, mutein thereof, for example but not limited nucleic acid sequences which encode the protein of SEQ ID NO: 1, or derivatives, analogues, variants or homologues thereof. The nucleic acid sequences can further include vector DNA, such that the coding region can be introduced into a host cell. Also encompassed are those complementary to DNA, mRNA, and tRNA encoding a clusterin protein of the present invention.

A number of viral vector systems are available including retroviral systems (see, e.g., Lawrie and Tumin, Cur. Opin. Genet. Develop. 3, 102-109, 1993); adenoviral vectors (see, e.g., Bett et al., J. Virol. 67, 5911, 1993); adeno-associated virus vectors (see, e.g., Zhou et al., J. Exp. Med. 179, 1867, 1994), viral vectors from the pox family including vaccinia virus and the avian pox viruses, viral vectors from the alpha virus genus such as those substantially similar to a region of the Sindbis and Semliki Forest Viruses (see, e.g., Dubensky et al., J. Virol. 70, 508-519, 1996), Venezuelan equine encephalitis virus (see U.S. Pat. No. 5,643,576) and rhabdoviruses, such as vesicular stomatitis virus (see WO 96/34625) and papillomaviruses (Ohe et al., Human Gene Therapy 6, 325-333, 1995); Woo et al., WO 94/12629 and Xiao Brandsma, Nucleic Acids. Res. 24, 2630-2622, 1996).

These vectors can be viral vectors such as adenovirus, adeno-associated virus, pox virus such as an orthopox (vaccinia and attenuated vaccinia), avipox, lentivirus, murine moloney leukemia virus, etc. Alternatively, plasmid expression vectors can be used.

Viral vector systems which can be utilized in the present invention include, but are not limited to, (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. In a preferred embodiment, the vector is an adenovirus. Replication-defective viruses can also be advantageous. The vector may or may not be incorporated into the cells genome. The constructs may include viral sequences for transfection, if desired. Alternatively, the construct may be incorporated into vectors capable of episomal replication, e.g EPV and EBV vectors.

Constructs for the recombinant expression of a clusterin protein as disclosed herein, such as SEQ ID NO: 1 will generally require regulatory elements, e.g., promoters, enhancers, etc., to ensure the expression of the construct in target cells. Other specifics for vectors and constructs are described in further detail below.

As used herein, a “promoter” or “promoter region” or “promoter element” used interchangeably herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

The term “regulatory sequences” is used interchangeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation.

Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

The term “operatively linked” as used herein refers to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined. In some embodiments, it can be advantageous to direct expression of a peptide immunogen in a tissue- or cell-specific manner.

In a specific embodiment, viral vectors that contain nucleic acid sequences encoding a clusterin protein n or fragments or derivatives or variants thereof are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding a clusterin protein or fragments or variants thereof are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

The production of a recombinant retroviral vector carrying a gene of interest is typically achieved in two stages. First, sequence encoding a peptide immunogen or fragments or derivatives or variants thereof can be inserted into a retroviral vector which contains the sequences necessary for the efficient expression of the metabolic regulators (including promoter and/or enhancer elements which can be provided by the viral long terminal repeats (LTRs) or by an internal promoter/enhancer and relevant splicing signals), sequences required for the efficient packaging of the viral RNA into infectious virions (e.g., a packaging signal (Psi), a tRNA primer binding site (−PBS), a 3[prime] regulatory sequence required for reverse transcription (+PBS)), and a viral LTRs). The LTRs contain sequences required for the association of viral genomic RNA, reverse transcriptase and integrase functions, and sequences involved in directing the expression of the genomic RNA to be packaged in viral particles.

Following the construction of the recombinant retroviral vector, the vector DNA is introduced into a packaging cell line. Packaging cell lines provide viral proteins required in trans for the packaging of viral genomic RNA into viral particles having the desired host range (e.g., the viral-encoded core (gag), polymerase (pol) and envelope (env) proteins). The host range is controlled, in part, by the type of envelope gene product expressed on the surface of the viral particle. Packaging cell lines can express ecotrophic, amphotropic or xenotropic envelope gene products. Alternatively, the packaging cell line can lack sequences encoding a viral envelope (env) protein. In this case, the packaging cell line can package the viral genome into particles which lack a membrane-associated protein (e.g., an env protein). To produce viral particles containing a membrane-associated protein which permits entry of the virus into a cell, the packaging cell line containing the retroviral sequences can be transfected with sequences encoding a membrane-associated protein (e.g., the G protein of vesicular stomatitis virus (VSV)). The transfected packaging cell can then produce viral particles which contain the membrane-associated protein expressed by the transfected packaging cell line; these viral particles which contain viral genomic RNA derived from one virus encapsidated by the envelope proteins of another virus are said to be pseudotyped virus particles.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Another preferred viral vector is a pox virus such as a vaccinia virus, for example an attenuated vaccinia such as Modified Virus Ankara (MVA) or NYVAC, an avipox such as fowl pox or canary pox. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In another embodiment, lentiviral vectors are used, such as the HIV based vectors described in U.S. Pat. Nos. 6,143,520; 5,665,557; and 5,981,276, which are herein incorporated by reference. Use of Adeno-associated virus (AAV) vectors is also contemplated (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient. Such cells, can be for example but are not limited to, cells in the blood and plasma, as well as bone marrow cells.

U.S. Pat. No. 5,676,954 (which is herein incorporated by reference) reports on the injection of genetic material, complexed with cationic liposome carriers, into mice. U.S. Pat. Nos. 4,897,355, 4,946,787, 5,049,386, 5,459,127, 5,589,466, 5,693,622, 5,580,859, 5,703,055, and international publication NO: WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein incorporated by reference) provide methods for delivering DNA-cationic lipid complexes to mammals. Such cationic lipid complexes or nanoparticles can also be used to deliver protein.

A gene or nucleic acid sequence can be introduced into a target cell by any suitable method. For example, an a nucleonic acid sequence encoding a clusterin protein or fragments or derivatives or variants thereof can be introduced into a cell by transfection (e.g., calcium phosphate or DEAE-dextran mediated transfection), lipofection, electroporation, microinjection (e.g., by direct injection of naked DNA), biolistics, infection with a viral vector containing a muscle related transgene, cell fusion, chromosome-mediated gene transfer, microcell-mediated gene transfer, nuclear transfer, and the like. A nucleic acid encoding a clusterin protein or fragments or derivatives or variants thereof are used can be introduced into cells by electroporation (see, e.g., Wong and Neumann, Biochem. Biophys. Res. Commun. 107:584-87 (1982)) and biolistics (e.g., a gene gun; Johnston and Tang, Methods Cell Biol. 43 Pt A:353-65 (1994); Fynan et al., Proc. Natl. Acad. Sci. USA 90:11478-82 (1993)).

In certain embodiments, a gene or nucleic acid sequence encoding a clusterin protein or fragments or derivatives or variants thereof can be introduced into target cells by transfection or lipofection. Suitable agents for transfection or lipofection include, for example, calcium phosphate, DEAE dextran, lipofectin, lipfectamine, DIMRIE C, Superfect, and Effectin (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999)).

Methods known in the art for the therapeutic delivery of agents such as proteins and/or nucleic acids can be used for the delivery of a polypeptide or nucleic acid encoding a clusterin protein or fragments or derivatives or variants thereof are used treating and/or preventing disease and disorders associated with TTR amyloidosis in a subject, e.g., cellular transfection, gene therapy, direct administration with a delivery vehicle or pharmaceutically acceptable carrier, indirect delivery by providing recombinant cells comprising a nucleic acid encoding a targeting fusion polypeptide of clusterin of the invention.

Various delivery systems are known and can be used to directly administer therapeutic clusterin protein as disclosed herein, or fragments or derivatives or variants thereof, e.g., encapsulation in liposomes, microparticles, microcapsules, recombinant cells capable of expressing the compound, and receptor-mediated endocytosis (see, e.g., Wu and Wu, 1987, J. Biol. Chem. 262:4429-4432). Methods of introduction can be enteral or parenteral and include but are not limited to intradermal, intramuscular, intraperitoneal, intravenous, subcutaneous, pulmonary, intranasal, intraocular, epidural, and oral routes. The agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, the implant being of a porous, non-porous, or gelatinous material, including membranes, such as sialastic membranes, fibers, or commercial skin substitutes.

In another embodiment, clusterin protein or fragments or derivatives or variants thereof as disclosed herein can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105).

Thus, a wide variety of gene transfer/gene therapy vectors and constructs are known in the art. These vectors are readily adapted for use in the methods of the present invention. By the appropriate manipulation using recombinant DNA/molecular biology techniques to insert an operatively linked nucleic acid sequence encoding a clusterin protein as disclosed herein, or fragments or derivatives or variants thereof, into the selected expression/delivery vector, many equivalent vectors for the practice of the methods described herein can be generated.

It will be appreciated by those of skill that cloned genes readily can be manipulated to alter the amino acid sequence of a clusterin protein or fragments or derivatives or variants thereof. The cloned nucleic acid sequence for a clusterin protein as disclosed herein can be manipulated by a variety of well known techniques for in vitro mutagenesis, among others, to produce variants of the naturally occurring human protein, herein referred to as variants or muteins or mutants of the peptide immunogen, which may be used in accordance with the methods and compositions described herein.

DNA encoding clusterin protein of the present invention, or a vector containing the same, can be packaged into liposomes. Suitable lipids and related analogs are described by U.S. Pat. Nos. 5,208,036, 5,264,618, 5,279,833 and 5,283,185. Vectors and DNA encoding an immunogen can also be adsorbed to or associated with particulate carriers, examples of which include polymethyl methacrylate polymers and polylactides and poly(lactide-co-glycolides), see, e.g., McGee et al., J. Micro Encap. (1996).

Gene therapy vectors or naked DNA can be delivered in vivo by administration to an individual patient, typically by systemic administration (e.g., intravenous, intraperitoneal, intranasal, gastric, intradermal, intramuscular, subdermal, or intracranial infusion) or topical application (see e.g., U.S. Pat. No. 5,399,346). Such vectors can further include facilitating agents such as bupivacaine (U.S. Pat. No. 5,593,970). DNA can also be administered using a gene gun. See Xiao Brandsma, supra. The DNA encoding an immunogen is precipitated onto the surface of microscopic metal beads. The microprojectiles are accelerated with a shock wave or expanding helium gas, and penetrate tissues to a depth of several cell layers. For example, The Accel™ Gene Delivery Device manufactured by Agracetus, Inc., (Middleton, Wis.) is suitable. Alternatively, naked DNA can pass through skin into the blood stream simply by spotting the DNA onto skin with chemical or mechanical irritation (see WO 95/05853).

In a further variation, vectors encoding clusterin protein or the present invention can be delivered to cells ex vivo, such as cells explanted from a subject (e.g., lymphocytes, bone marrow aspirates, tissue biopsy, induced pluripotent stem cells (iPSCs)) or universal donor hematopoietic stem cells, followed by reimplantation of the cells into a subject, usually after selection for cells which have incorporated the vector, and expression of the nucleic acid encoding the clusterin protein of the present invention. As discussed infra, the iPSC can be genetically modified, e.g. ex vivo to express a clusterin protein or agonist of clusterin protein activity as disclosed herein, and then subsequently implanted back into a subject in accordance of the method and compositions as disclosed herein for the treatment of diseases and disorders associated with amyloidosis, e.g. TTR amyloidosis.

### Kits

In some embodiments, the present invention further includes a kit for use in a method of measuring the amount of clusterin in a biological sample, where the kit comprises a binding partner, as described above, in an assay-compatible format, for interaction with a clusterin protein present in the biological sample. Thus, in some embodiments, it is contemplated within the invention to use an antibody chip or array of chips, capable of measuring clusterin levels can be used.

In some embodiments, a kit for use in a method or system for measuring the amount of clusterin is a biological sample from the subject is an immunoassay, for example but not limited to immunofluorescent assay, ELISA, chemiluminescent assay, and in some embodiments, the kit can optionally include instructions for measuring clusterin levels.

In some embodiment, a kit can comprise a reference sample, e.g., a control reference sample from a healthy subject (e.g., a negative control), and in some embodiments, a positive control sample (e.g., obtained from a subject with low clusterin levels, or having amyloidic cardiopathy).

Other aspects of the invention provides kits or pharmaceutical packages that include a clusterin agent for therapeutic use for treatment of a subject identified to have cardiac amyloid deposits by the methods and systems as discussed herein. In addition to one or more clusterin agents in the form of, for example, tablets, capsules, or lyophilized powders, the kits or packages can include instructions for using the clusterin agents in the treatment of cardiac amyloid deposits or amyloidotic cardiomyopathy and conditions associated with low serum clusterin levels. A clusterin agent can be provided in the kits or packages in a bottle or another appropriate form (e.g., a blister pack). Optionally, the kits or pharmaceutical packages can also include other pharmaceutically active agents (see, e.g., the agents listed above, such as anti-obesity agents), and/or materials used in administration of the drug(s), such as diluents, needles, syringes, applicators, and the like.

Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference.

### EXAMPLES

The examples presented herein relate to the design and use of clusterin protein or analogues of clusterin activity for use in the methods and compositions as disclosed herein for the treatment and/or prevention of subjects with TTR amyloidosis. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Materials and Methods

Expression and Preparation of TTR.

Human transthyretin cDNA was obtained from the NIH Mammalian Gene Collections. TTR variants V30M and L55P were generated using a Quikchange mutagenesis kit (Stratagene). Mutagenesis primers for TTR vari-ants were V30M-F: 50-cctgccatcaatgtggccat gcatgtgttcagaaagg, V30M-R: 50-cctttctgaacacatgcatgg ccacattgatggcagg, L55P-F: 50-ccagtgagtctggagagccgc atgggctcacaactg, and L55P-R: 50-cagttg tgagcccatgcgg ctctccagactcactgg. For purification of wild-type TTR and TTR variant proteins, a GST-fusion protein system was used. The coding sequences of wild-type TTR and each TTR variant were amplified by PCR using primer set F: 50-gcggccctacgggca ccggtg and R: 50-gctcattccttgggattggtg, and subcloned into EcoRI and XhoI sites of pGEX4T-1. The plasmids were transformed into BL21 (DE3) Escherichia coli. In each case, transformed cells were cultured to A 600, 0.5-0.6 and 1 mM IPTG added for 4 h at 37° C. The cells were harvested after induction and disrupted by lysis buffer (150 mM NaCl, 5 mM EDTA, 0.5% NP40, and 50 mM Tris pH 7.6). After centrifugation, the supernatant was mixed with Sepharose 4B agarose (GE Healthcare) for 1 h at 4° C. and applied to column. The agarose beads were washed with 4 volumes PBS and then thrombin (Sigma) digested for overnight at room temperature. TTR solution was concentrated using a 1 kD cut-off Centriprep filter (Millipore).

Preparation of Baculovirus and Purification of Clusterin.

Rat clus-terin cDNA provided by IS Park (Inha University, Korea) was amplified using Clu-F; 50-acaaaaaagcaggctccaccatgaaga ttctcctgctgtgtgtg and Clu-R; 50-ttgtacaagaaagct gggttttccatgcggcttttcctgcggt. The shuttle clone of pEntr-Clusterin was made by in vitro recombination between the PCR products and pDONR207 vector with BP clonase (Invitrogen). For a recombinant baculovirus preparation, BacHTS System (Newgex, Korea) was used following the protocols provided by manufacturer. In brief, the entry clone and the baculovirus genomic DNA vector were combined in vitro with LR clone (Invitrogen). The reaction product was transfected into Sf21 cells with Lipofectin reagent to produce the recombinant baculovirus bearing C-terminally V5 and 6×His-tagged clusterin. Three days after virus infection, the culture media was centrifuged at 3500 rpm or 15 min to remove cell debris. The supernatant was mixed with Ni-NTA resin in 50 mM sodium phosphate buffer (pH 7.6) for 2 h and applied to the column. The column was washed with wash buffer (20 mM Imidazole, 50 mM sodium phos-phate buffer (pH 7.6), 300 mM NaCl) and then eluted with elution buffer (250 mM Imidazole, 50 mM sodium phosphate buffer (pH 7.6), 300 mM NaCl)

GST-Pull-Down Assay.

GST-fusion proteins bound to glutathione-Sepharose 4B were used as affinity matrix for clusterin binding experiments. Equal amounts of GST and GST-fusion proteins were incubated for 12-24 h at room temperature with purified recombinant clusterin in PBS or acetate buffer (200 mM acetate buffer, pH 4.4, 100 mM KCl, and 1 mM EDTA). After incubation, the beads were washed with 2-3 volumes of PBS three times. The bound clusterin was resolved by SDS-PAGE and transferred to nitrocellu-lose membranes. The membranes were incubated with polyclonal antibodies against clusterin (C-18, Santa Cruz Biotechnology) or transthyretin (Dako). The bands were visualized by LAS-4000 (Fujifilm).

In Vitro Fibril Formation Assay.

Fibril formation for serum TTR protein (Calbiochem), wild-type TTR and TTR variants were performed as described [23]. Briefly, 0.44 mg/ml and 0.88 mg/ml of wild-type TTR and TTR variants in phosphate buffer (20 mM phosphate buffer pH 7.6, 100 mM KCl, and 1 mM EDTA) were combined with acetate buffer in the presence or absence of clusterin (0.15 mg/ml or 0.075 mg/ml) and incubated at 37° C. for the times indicated. The tubes were vortexed for 5 seconds and the turbidity measured at 450 nm. For cytotoxicity experiments, 0.88 mg/ml of TTR proteins in phosphate buffer (20 mM phosphate buffer pH 7.6, 100 mM KCl, and 1 mM EDTA) were combined with acetate buffer in the presence or absence of clusterin and was incubated for 1 day at room temperature and centrifuged at 15,000 rpm for 1 h. The pellet fraction of TTR proteins were suspended with culture media and added to the SH-SY5Y cells.

Glutaraldehyde Cross-Linking.

The cross-linking method [20] was performed to check the tetrameric structure of TTR proteins in the presence or absence of clusterin. Glutaraldehyde (25%) was added to the protein solution (10% v/v), and incubated for 4 min. The reaction was stopped by the addition of NaBH₄ (7% in 0.1 M NaOH) and mixed with 5% SDS sample buffer. The extent of TTR tetramer was visualized by Western blot analysis as described above.

Cell Culture and MIT Assay.

Human SH-SY5Y neuroblastoma cells (ATCC, CRL-2266) were cultured at 37° C. in a humidified atmosphere with 5% CO2 in DMEM/F12 medium containing 50 U/mL penicillin and 50 lg/mL streptomycin, supplemented with 10% (v/v) fetal bovine serum. Cells were plated to a 96-well plate at 5×103 density and left to attach overnight. The cells were then cultured as above for 48 h with or without additives and subjected to the MTT assay. Cell viability was measured by MTT (3[4,5-dimethylthiazol-2-yl]-2,5-diphenyltetrazolium bromide) assay.

Transfection with Clusterin cDNA.

A plasmid was constructed that contains a truncated form of clusterin cDNA with deletion of the sequence responsible for cross-membrane transport. Transfection of human embryonic 293 cell line with this plasmid leads to intracellular overexpression of a non-secreted form of clusterin. The clusterin gene is under control of a CMV promoter with tetracycline-sensitive elements. Transfected cells do not express clusterin until they are exposed to tetracycline. This plasmid will be used to deliver the truncated clusterin gene into stem cells. The cells will be selected using G418 as the plasmid contains a neomycin-resistant gene.

Detection of Clusterin Expression at mRNA and Protein Levels.

Two methods can be used for determination of clusterin mRNA levels. The first method is Real-time RT-PCR using a quantitative, real-time PCR cycler (Smart Cycler, Cepheid, Suwance, Ga.). The second method is RNase protection assays. Total RNA was isolated from cultured cells or tissues using a RNA isolation kit from Promega. For determination of clusterin protein levels, immunoblotting with anti-clusterin was performed.

Clusterin Levels in Blood and in the HDL Fraction.

To evaluate the levels of clusterin in the blood, an ELISA method can be used with monoclonal antibody against clusterin. Serum was prepared from the blood samples. Cholesterol levels, lipoprotein profiles and clusterin concentrations were determined respectively. In brief, 100 μl of serum diluted in PBS was incubated in a 96 well plate coated with a rabbit polyclonal antibody to clusterin. After incubation and washing in PBS, bound clusterin was detected by incubating with mouse monoclonal antibody to clusterin. Goat anti-mouse IgG conjugated with peroxidase was used as the second antibody. Cholesterol and HDL was determined in the laboratory of Department of Laboratory Medicine. The ratio of clusterin vs. HDL was calculated after normalization with the lipid content. In addition to ELISA, immunoblotting assays were performed to verify the results from ELISA. 20 μg/lane of serum proteins was loaded into 10% SDS-PAGE. After electrophoresis, protein bands were transferred to a membrane and stained with anti-clusterin. Immunoperoxidase method was used to detect clusterin bands.

Expression of Clusterin Protein and mRNA in the Heart and Other Tissues.

Quantitative morphological analysis of clusterin expression in the heart and aorta can be conducted using immunohistochemistry using monoclonal anti-clusterin. The sections were stained by immunostaining with anti-clusterin. Immunoblotting assays can be conducted in order to backup the data from immunohistochemistry. Total proteins extracted from plaques were examined with anti-clusterin using the method described above. Clusterin mRNA can be evaluated using real time RT-PCR. The real time RT-PCR is a very useful method which is highly sensitive and reliable. Total RNA was isolated from plaques and converted into cDNA by reverse transcription followed by amplification with Taq polymerase. A real-time PCR cycler can be used to quantitatively determine the levels of clusterin mRNA.

Study Cohorts.

Patient information and biological samples were obtained from the Boston University Amyloid Treatment and Research Program repository with the approval of the Institutional Review Board at the Boston University Medical Campus in accordance with the Declaration of Helsinki Clinical data included details of history, physical examination, and routine laboratory studies. Patients were determined to have systemic amyloidosis by the presence of congophilic fibrillar deposits in fat aspirates or tissue biopsies. Typing was determined by a combination of immunochemical, biochemical, and genetic techniques. The diagnosis of AL was established by the presence of a plasma cell dyscrasia identified by clonal plasma cells in a bone marrow biopsy and evidence of a monoclonal immunoglobulin LC by serum and urine immunofixation electrophoresis and/or free light chain nephelometry. Identification of a pathologic TTR gene mutation and/or mutant protein indicated ATTR amyloidosis. In SSA, the diagnosis is made when testing for AL and AF are negative and there is immunological or biochemical proof of TTR in the amyloid deposits. Cardiac function was assessed by echocardiogram. Left ventricular mass (LVM) was derived from the formula described by Devereux et al.¹⁴ Patients were identified as having cardiac involvement if they had clinical symptoms of congestive heart failure (defined as New York Heart Association Functional Class >1) and/or septal or ventricular wall thickening >12 mm on echocardiogram without a history of hypertension. Electrocardiogram (ECG) and brain natriuretic peptide (BNP) were also determined.

Commercial control sera collected from healthy, consented, paid human donors from FDA-licensed and inspected donor centers, was purchased from Bioreclamation, Inc. (Westbury, N.Y.). Sera from patients with ischemic and non-ischemic CMP, left ventricular ejection fraction (LVEF) <40% and no amyloid disease served as a second control group in the study.

Antibodies and Other Reagents.

The polyclonal rabbit anti-human CLU (H-330), monoclonal mouse anti-human CLU (CLI-9), and goat anti-rabbit IgG-HRP mouse/human adsorbed antibody solutions were purchased from Santa Cruz Biotechnology (Santa Cruz, Calif.); polyclonal rabbit anti-human-prealbumin (TTR) (DakoCytomation, Glostrup, Denmark), and polyclonal goat anti-human-kappa light chain conjugated to horseradish peroxidase (HRP) from Novus (Littleton, Colo.). Envision+ System-HRP labeled polymer anti-rabbit secondary antibody, proteinase K, Dako antibody diluent, and TBST were from DakoCytomation (Glostrup, Denmark). Congo red, hematoxylin, citra Plus®, and Power Block® solutions (BioGenex, San Ramon, Calif.). EM polyclonal goat anti-rabbit IgG: 10 nm antibody solution (British BioCell International, Cardiff, UK), Lowicryl® K4M resin, and formvar film 150 square mesh nickel grids were all acquired through Electron Microscopy Sciences (Hatfield, Pa.). All other chemicals used were from Fisher (Fairlawn, N.J.) or Sigma (St. Louis, Mo.) and were of the highest grade available.

Congo Red Staining & Immunohistochemistry.

Formalin-fixed, paraffin-embedded cardiac tissues were serially sectioned to 5 um thickness and processed for Congo red staining and immunohistochemical analysis. For Congo red staining, deparaffinized sections were counterstained with Mayer's hematoxylin for 1 min, placed in alkaline 80% alcohol/NaCl for 20 min, stained in alkaline Congo red for 20 min, rinsed in ethanol and xylene, and observed by polarized light microscopy.

Indirect immunohistochemical techniques with rabbit anti-human primary antibodies and goat anti-rabbit secondary antibodies conjugated to horseradish peroxidase (HRP) were used for analyses of CLU and TTR in tissue sections, while a direct method performed with anti-human free kappa LC antibodies conjugated to HRP was used for kappa LC evaluation. For studies of CLU and LC, slides were initially boiled in antigen retrieval citra Plus® solution and cooled for 20 min to room temperature; in TTR analyses, slides were pre-treated in 6M guanidine, pH 7.4 for 1 hr. Subsequently, all slides were washed 3×s with dH₂O and rinsed using TBST wash buffer. Tissue sections were applied to slides, blocked for 10 min with fresh Power Block® and rinsed with TBST wash buffer using the Dako Autostainer Plus® automated system from DakoCytomation (Glostrup, Denmark). Using Dako diluent buffer, stock antibody solutions were diluted 1:50 for CLU, 1:1000 for TTR, and 1:100 for kappa LC-HRP antibodies; sections were incubated in 200 μL of appropriate primary antibody dilutions for 30 min. For the TTR studies, sections were treated with proteinase K for 5 min and washed prior to incubation in primary antibody. For the analyses of CLU and TTR, 200 μL of Envision+System-HRP labeled polymer anti-rabbit secondary antibody was applied for 30 min, washed with TBST, and treated with diaminobenzidine for 5 min. Sections were counterstained with Harris' modified hematoxylin. Imaging was performed under bright field microscopy using a SPOT Insight camera with SPOT Advanced 4.6 software.

Immunogold Labeling & Electron Microscopy.

Frozen tissues were cut to no greater than 1 mm thickness and fixed overnight in 4% paraformaldehyde at 4° C. Sections were washed in sodium cacodylate buffer (Electron Microscopy Sciences, Hatfield, Pa.) and dehydrated in 50%, 75%, and 90% dimethylformamide solutions. Tissues were embedded in fresh Lowicryl® resin and block ultra-thin sections placed on nickel grids.

Tissue sections were blocked for 1 hr with 1.0% BSA, 5.0% normal goat serum in 0.05M Tris, pH 7.4. Stock primary antibody solutions were diluted with 0.5% BSA, 0.1% fish gelatin, 0.05% tween in 0.05M Tris, pH 7.4 as follows: 1:5 for CLU, 1:300 for TTR, and 1:4000 for kappa free LC antibodies. Samples were incubated with appropriate primary antibody dilutions overnight at 4° C., rinsed, and treated with a 1:10 dilution of 10 nm immunogold conjugated to goat anti-rabbit IgG for 1 hr. Grids were rinsed and negatively stained using 4% uranyl acetate and 0.4% lead citrate. Immunogold labeling was viewed at 80,000× magnification using Jeol Jem-1011 electron microscope (Peabody, Mass.) at 80 kV. Electron micrographs were acquired using the Gatan ES 1000W Erlangshen CCD camera and Gatan digital micrograph software (Pleasanton, Calif.).

Tissue Extraction and Immunoblot Analysis.

Briefly, biospecimens were washed using cold 12 mM phosphates, 137 mM NaCl, 2.7 mM KCl, pH 7.4, and nuclease free H₂O (Thermo Fisher). Next, 7M urea, 2M thiourea, 65 mM DTT, and 4% CHAPS was added at 300 μL/100 mg of tissue. Samples were homogenized using the TissueRuptor (Qiagen, Venlo, Netherlands) and centrifuged for 1.5 h at 4° C. and 25,000 rcf. Supernatant was transferred to a new tube and centrifuged 2×s for 30 min at 4° C. and 25,000 rcf. Next, supernatant whole protein extract concentrations were determined and samples stored at −80° C. Equal amounts of total protein were loaded per well for each blot as follows: CLU, 40 μg; TTR, 2 μg; Kappa LC, 1 μg. Extracts were electrophoresed under reducing conditions at 80V for 2 h using 8-16% gradient Tris-HCl SDS-gel and following transfer membranes were probed using the Snap i.d. system (Millipore, Billerica, Mass.).

Serum CLU Measurement by Indirect Capture ELISA.

High binding 96-well microplates (R&D Systems, Minneapolis, Minn.) were coated overnight at 4° C. with 100 μL of 62.5 ng/mL of mouse monoclonal anti-human CLU capture antibody and blocked with Blocker® Casein in PBS solution (Thermo Scientific, Rockford, Ill.). Serum samples were diluted to 1:40 in 0.1% BSA, 10% Blocker® Casein in PBS, 0.05% surfact-amp 20® (Thermo Scientific, Rockford, Ill.), in PBS and 100 μL applied to microplates in triplicate. Samples were incubated for 2 hrs rotating at 90 rpm. Standard curves were generated for independent microplates using rCLU (R&D Systems, Minneapolis, Minn.). Plates were washed and 100 μL of 2.5 μg/mL rabbit polyclonal anti-human CLU primary detection antibody added to each well. Following 2 hr incubation, 100 μL of 80 ng/mL preadsorbed goat anti-rabbit-secondary detection antibody was added and rotated for 2 hr at 90 rpm. ELISA solutions were developed for 20 min using 100 μL of an H₂O₂ and 3,3′,5,5′-tetramethylbenzidine chromagen system (R&D Systems, Minneapolis, Minn.); development was halted with 100 μL of 4 N sulfuric acid. Absorbance values were measured at 450 nm and 570 nm using a PowerWaveX spectrophotometer (BioTek, Winooski, Vt.). Standard curves and final serum concentrations were determined by a 4-parameter logistic curve fit using KC4 software (BioTek, Winooski, Vt.).

Statistical Analysis.

Data from the ELISA measurements of serum CLU and echocardiography were analyzed using GraphPad Prism 5 software (La Jolla, Calif.). One way analysis of variance and Tukey-Kramer multiple comparison post-ANOVA, and Pearson or Spearman correlation tests were conducted to determine significance. Continuous variables are described as mean±standard error. Statistical significance was assigned by the criterion of p<0.05.

### Representative Example

Recent reports have demonstrated that all disease-associated extracellular amyloid deposits co-localized with clusterin, which is proposed to form part of an extracellular protein quality control system[10,11]. Clusterin is a secreted, multifunctional glycoprotein that is involved in various physiological processes including cell aggregation, proliferation, and differentiation [12]. Recently, the emerging role of clusterin is that of performing quality control in extracellular space by capturing improperly folded proteins [13,14]. Particularly, clusterin has been reported to interact with amyloid-forming species including amyloid β peptide, apolipoprotein C-II and prion neuropeptide 106-126, and to suppress amyloid fibril formation and cytotoxicity [15-18]. Thus, the chaperone action of clusterin in vivo should be ‘protective’ by complexing with stressed or partly unfolded proteins and solubilizing these amyloid deposits.

Furthermore, one characteristic feature of clusterin is that chaperone activity is strongly increased under acidic conditions [19]. Actually, enhanced binding of clusterin to the heat-stressed alcohol dehydrogenase and lysozyme was observed at low pH. This chaperone property of clusterin may provide clues for studying the inhibition of TTR amyloidoses because TTR amyloid fibril formation may initiate in the acidic environments of endosomes or lysosomes [20-22], even though TTR amyloid deposition is known to occur in extracellular spaces. For that reason, Lee et al., have previously reported that clusterin may affect TTR amyloidoses occurring under acidic conditions, and cytotoxicity of FAP variants V30M and L55P TTR.

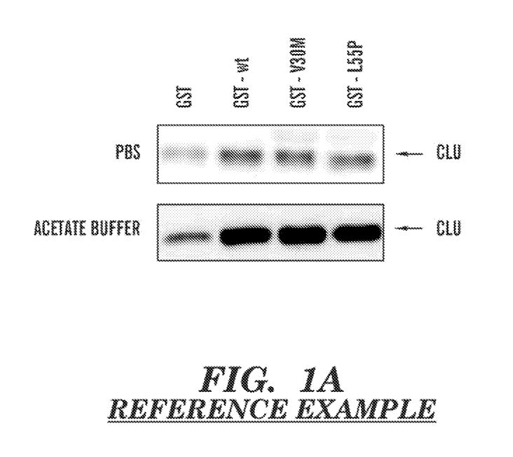

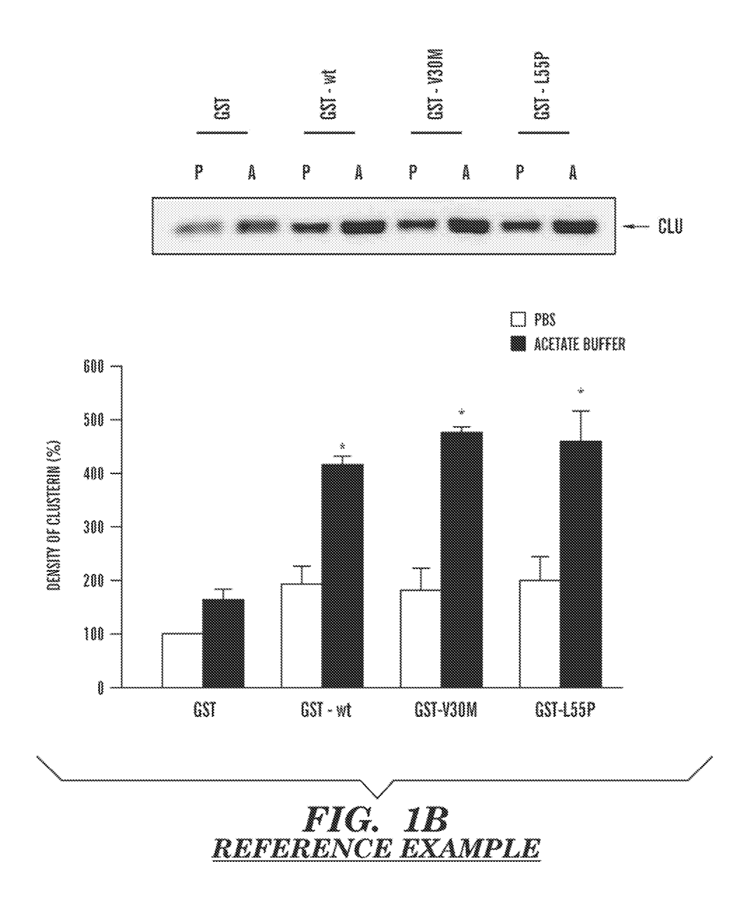

Activation of clusterin by acidic conditions increases its binding to wild-type TTR and TTR variants V30M and L55P. To explore the potential role of clusterin in TTR amyloidosis, the interaction between clusterin and TTR proteins including wild-type TTR (wt-TTR) and TTR variants V30M and L55P was assessed using a GST pull-down assay. Purified GST, GST-fusion wt-TTR and TTR variants V30M and L55P were incubated with purified recombinant clusterin for 12 h in vitro (FIG. 1A). Lee et al., reported that clusterin has weak binding activity to GST itself but binds more strongly to GST-fusion wt-TTR and TTR variants V30M and L55P in PBS. This enhanced binding of clusterin to GST-fusion wt-TTR and TTR variants V30M and L55P in PBS may explain a possible interaction of clusterin-TTR in physiological conditions. In particular, the binding of clusterin to wt-TTR and TTR variants under acidic conditions (pH 4.4) was much greater than that in PBS (FIG. 1). Following 24-hr incubation, Lee et al., reported that there was a nearly 2-fold increase in the binding of clusterin to wt-TTR and TTR variants (FIG. 1B). This result suggests that clusterin may have a potential role in TTR amyloidosis occurring under acidic conditions.

Clusterin Inhibits Amyloid Fibril Formation of TTR In Vitro.

To further understand the effect of clusterin on amyloid fibril formation of TTR, Lee et al., reported the extent of TTR amyloid fibril formation in the presence of clusterin by monitoring turbidity (FIG. 2A). Lee et al., measured amyloid fibril formation of TTR at 0.22 mg/ml or 0.44 mg/ml TTR because TTR is found in the range of 0.1-0.4 mg/ml concentration in human plasma. The extent of amyloid fibril formation of V30M and L55P was reported to be much greater than that exhibited by wt-TTR at both 0.22 mg/ml and 0.44 mg/ml (FIG. 2A).

Lee et al., also selected a concentration of 0.22 mg/ml wt-TTR and TTR variants V30M and L55P to measure the efficacy of clusterin as an inhibitor of amyloid fibril formation of TTR. Lee et al., reported that addition of clusterin to TTR variants showed a dose-dependent decrease in amyloid fibril formation (FIG. 2B). Lee et al., also reported that 0.15 mg/ml of clusterin added to the V30M TTR was sufficient to reduce fibril formation to levels similar to that of wt-TTR, whereas the same amount of clusterin moderately inhibited amyloid fibril formation of the L55P TTR, and suggested that clusterin may be an extracellular molecular chaperone could be a functional inhibitor of TTR amyloidosis.

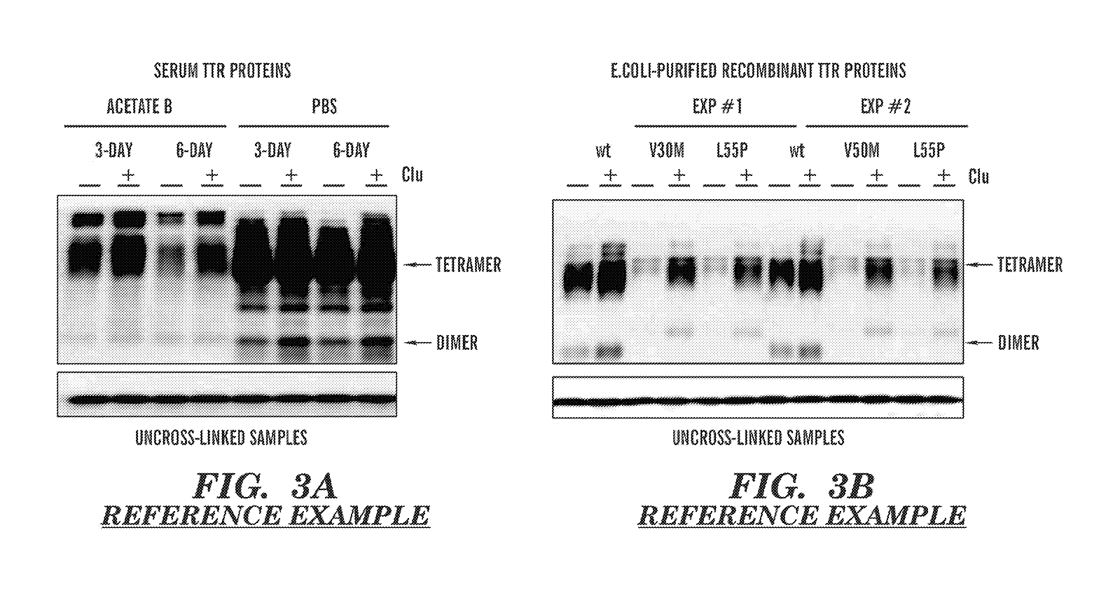

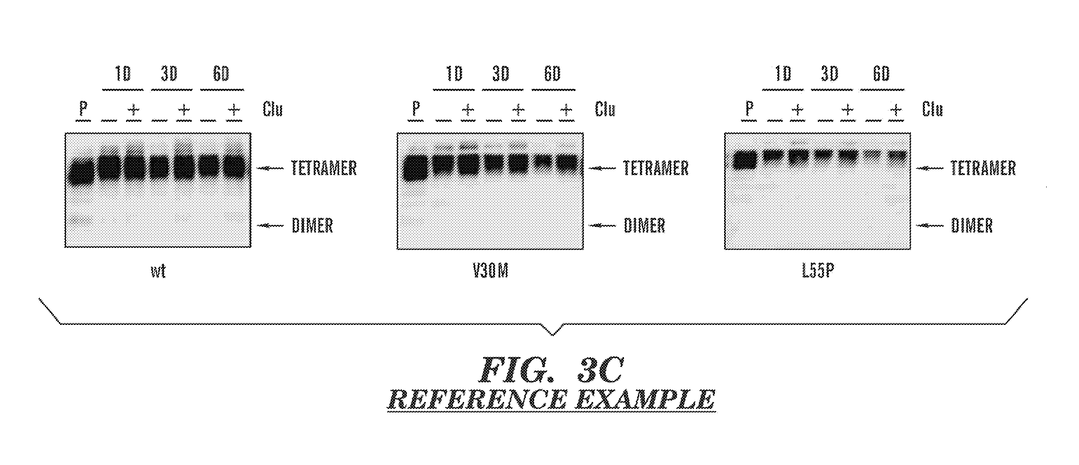

Clusterin increases TTR structural stability. In order to examine how clusterin acts on fibril formation of TTR, Lee et al., used a cross-linking assay to measure TTR structural stability under acidic conditions. Serum TTR proteins (0.22 mg/ml) from human plasma were incubated with 0.15 mg/ml of clusterin in acetate buffer (pH 4.4) for 3-6 days, cross-linked, and then the extent of TTR tetramers was examined by Western blot analysis. Lee et al., reported that serum TTR proteins from human plasma showed a time-dependent decrease in tetrameric and dimeric forms of TTR in acetate buffer (pH 4.4), but these levels of TTR were significantly maintained in the presence of clusterin under acidic conditions (FIG. 3A). Lee et al., also assessed the structural stability of purified recombinant wt-TTR and TTR variants V30M and L55P in the presence of clusterin, and reported that both TTR variants V30M and L55P showed much faster dissociation than wt-TTR under acidic conditions, resulting in severe loss of the tetrameric form of the TTR variants. Lee et al., reported that this dissociation was significantly prevented by clusterin, showing increased levels of tetrameric and dimeric forms of TTR variants (FIG. 3B). Lee et al., reported that since the TTR variants quickly dissociate into monomers under acidic conditions (pH 4.4), they were unable to check the structural stability of TTR variants over a long period. Therefore, they used mildly acidic condition (pH 5.0) to observe the structural stability of TTR variants with time. Lee et al., then reported that clusterin functioned to significantly stabilized the tetrameric structure of TTR variants over 6 days in vitro (FIG. 3C), and reported that that clusterin suppresses TTR amyloidosis by stabilizing the tetrameric structure of TTR.

Clusterin Decreases Neurotoxicity Induced by Fibrils of TTR Variants.

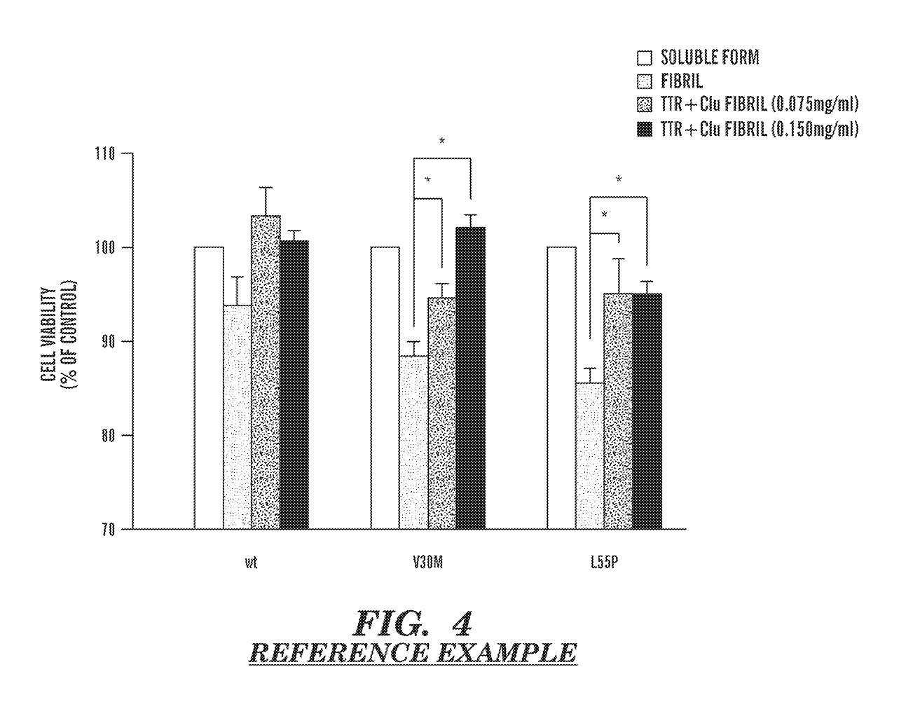

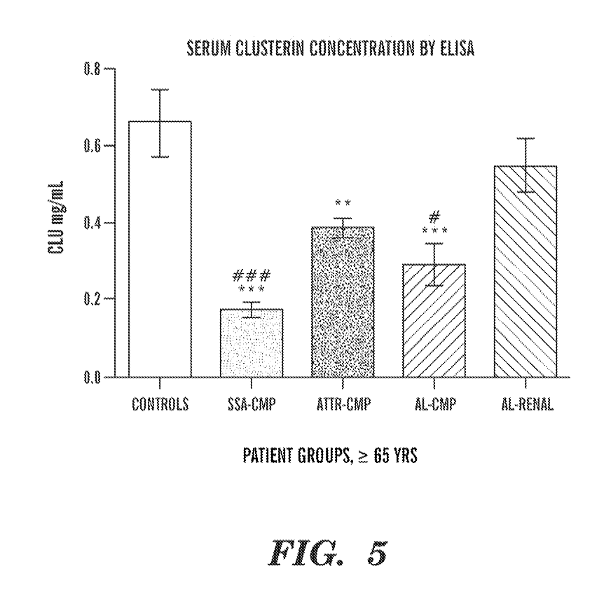

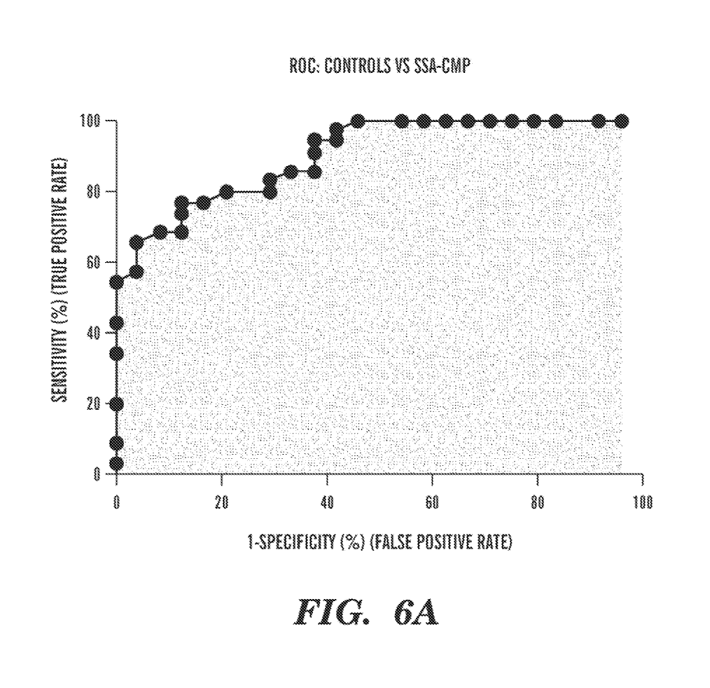

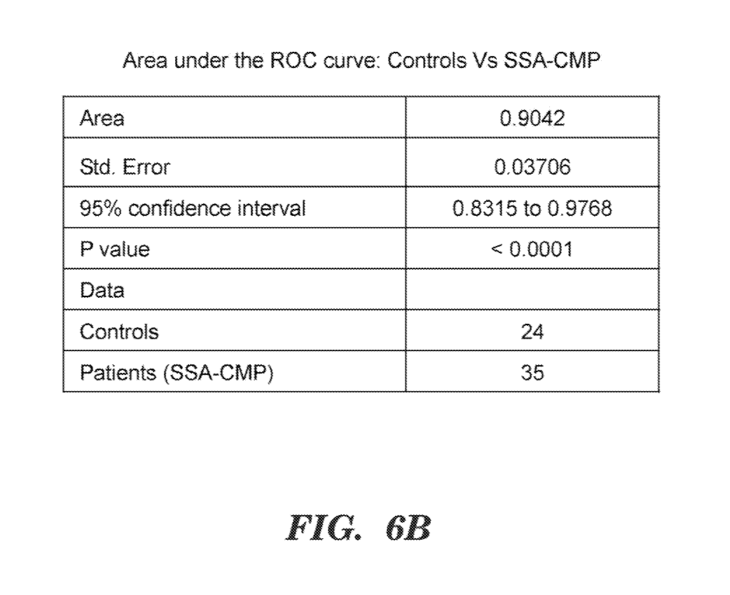

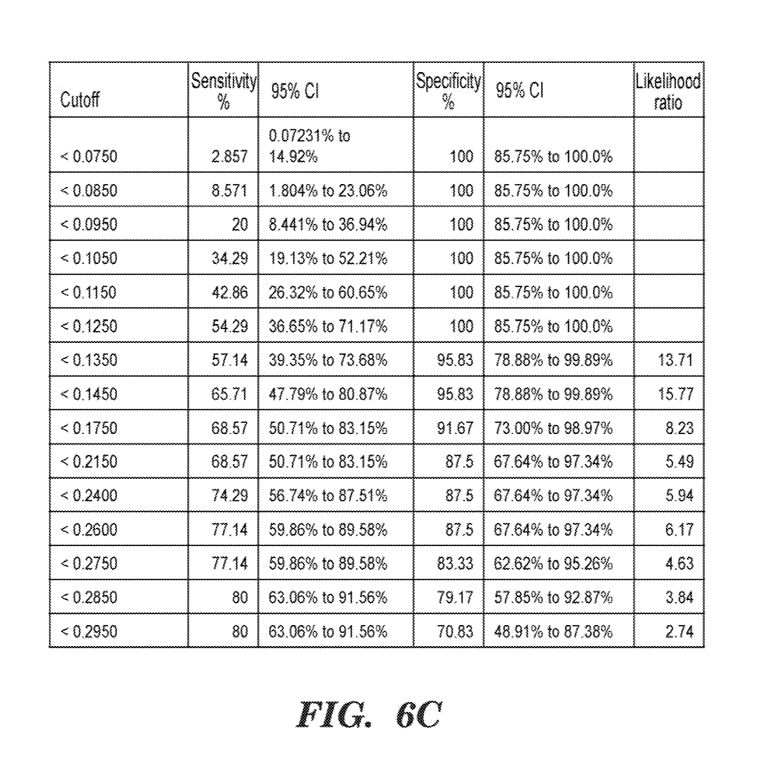

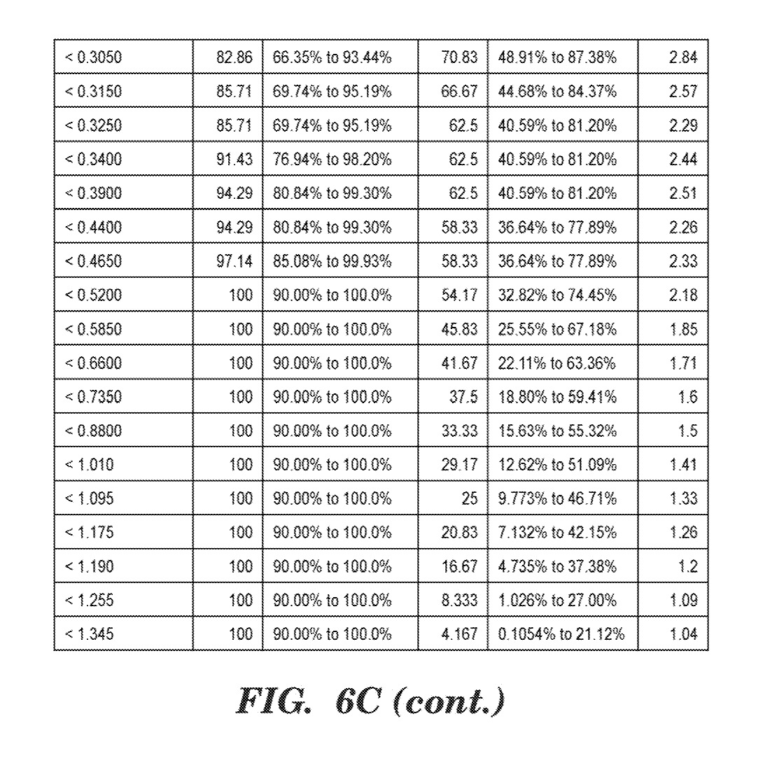

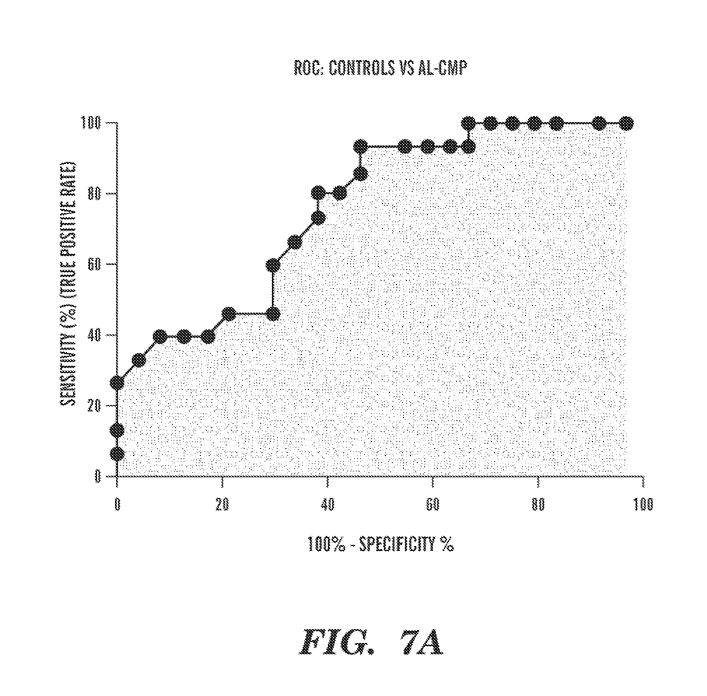

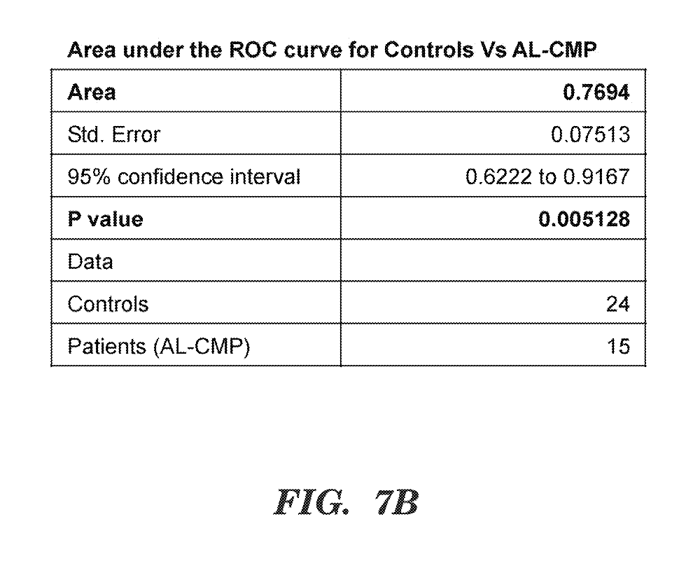

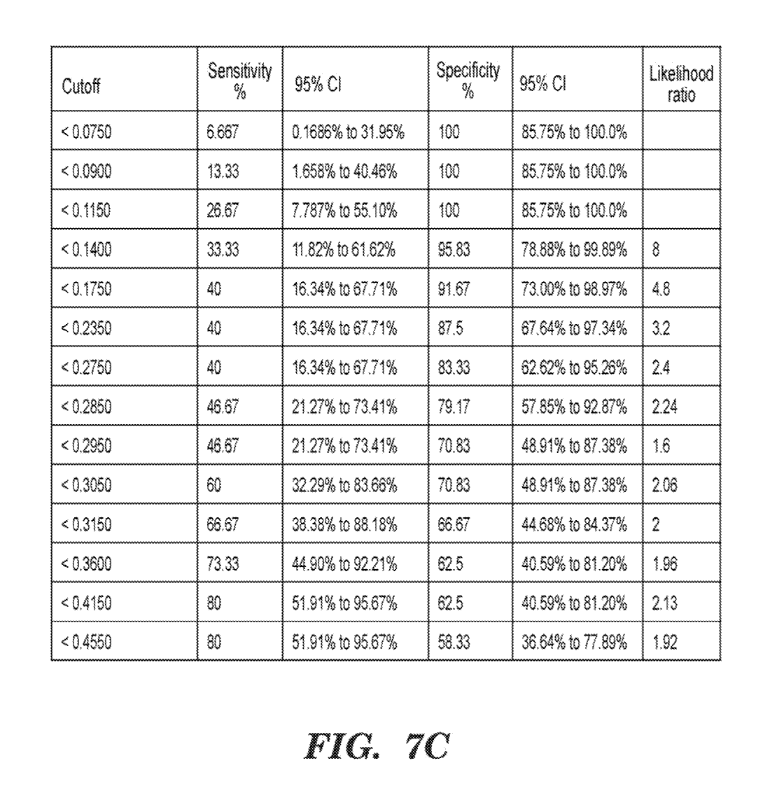

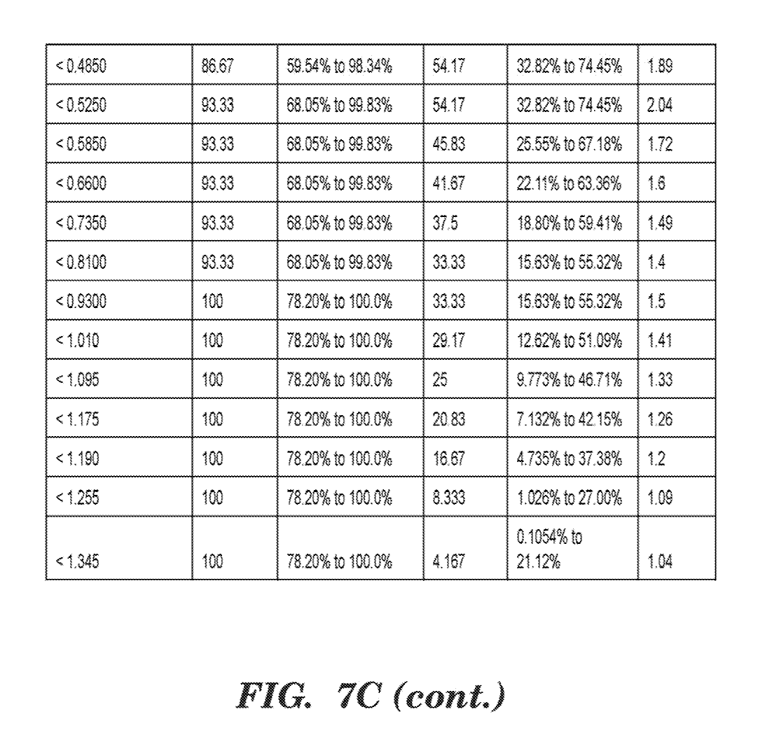

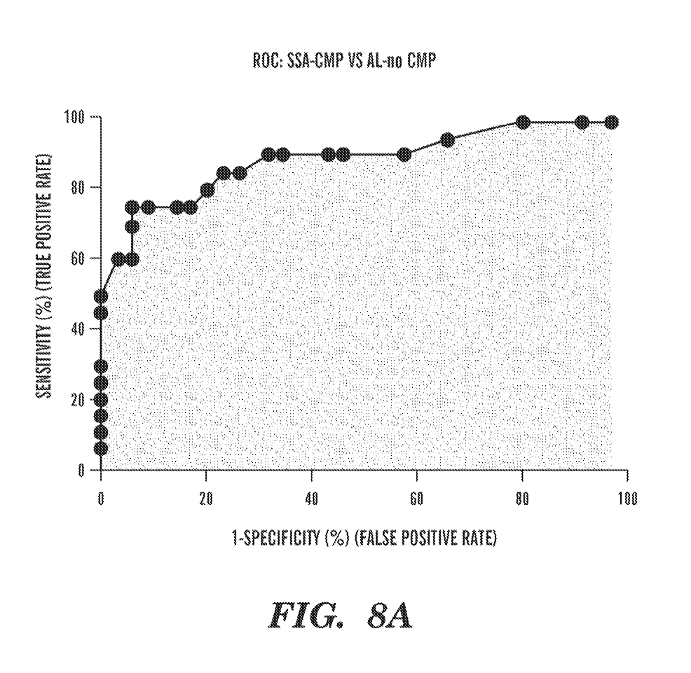

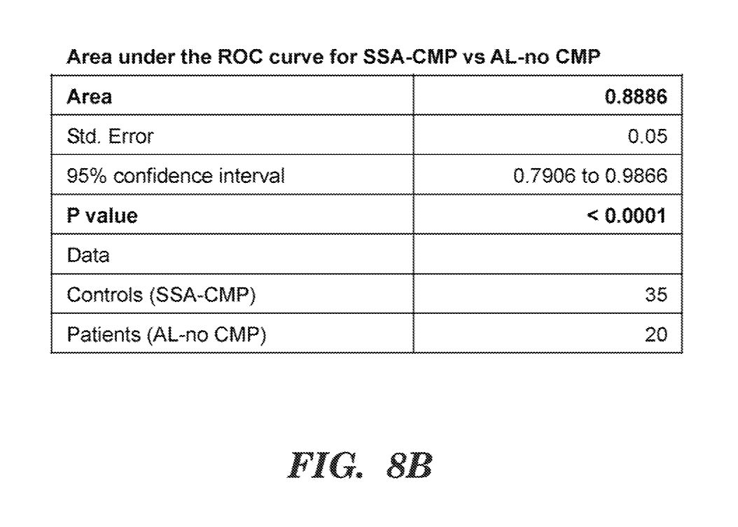

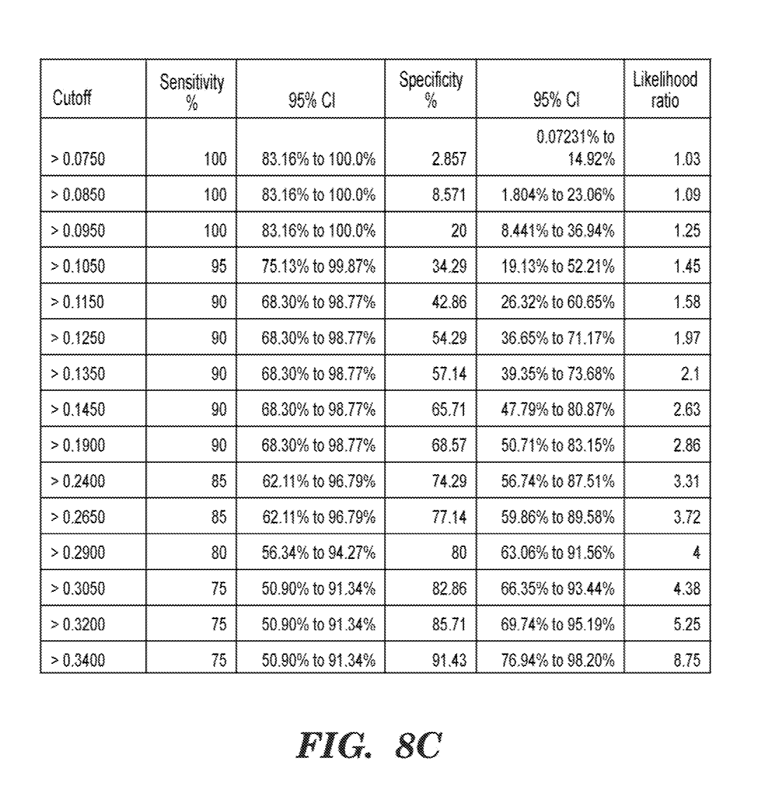

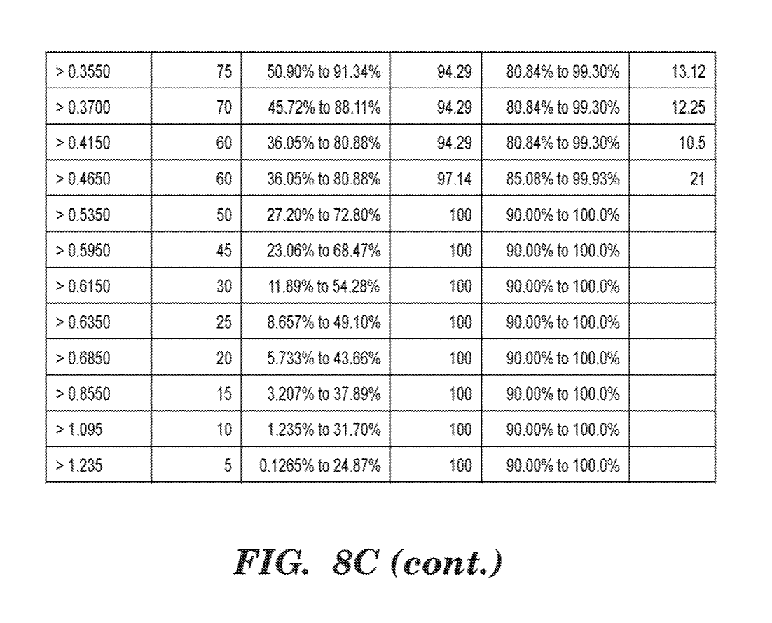

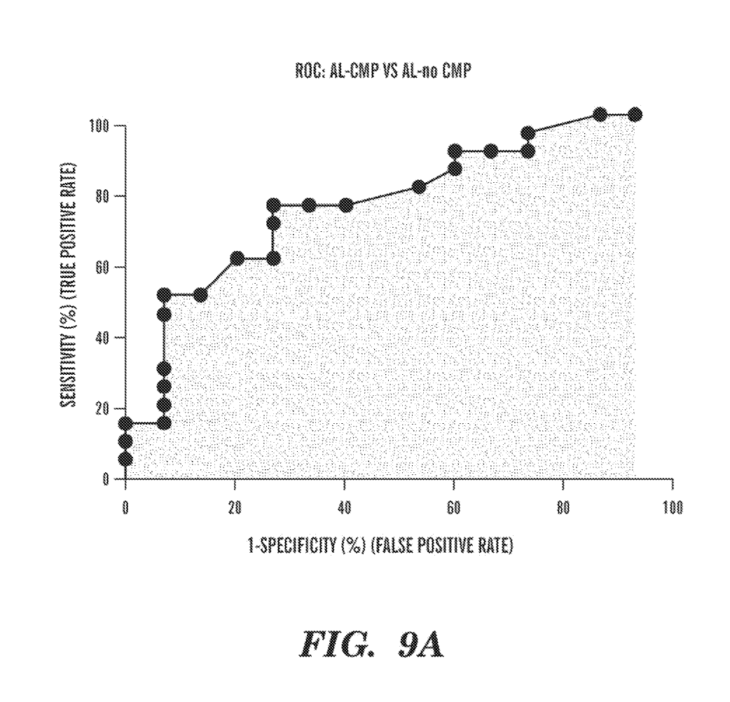

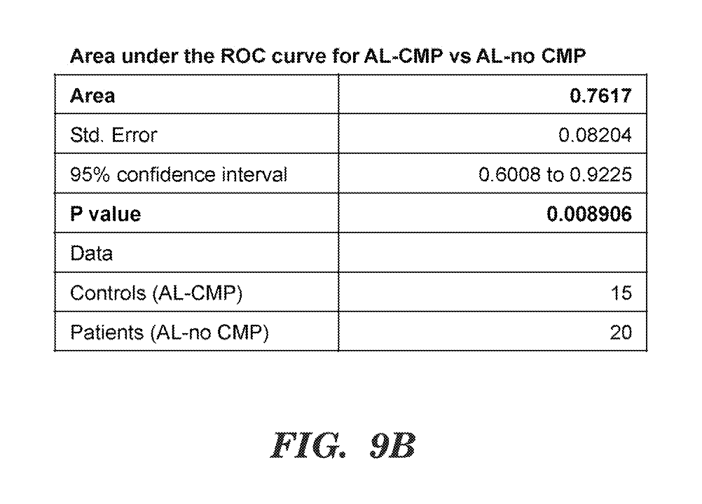

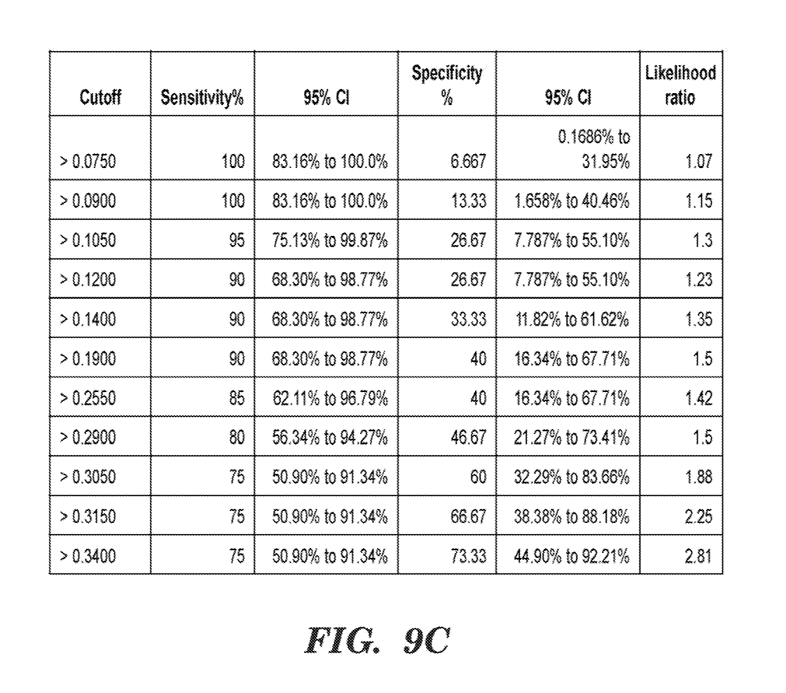

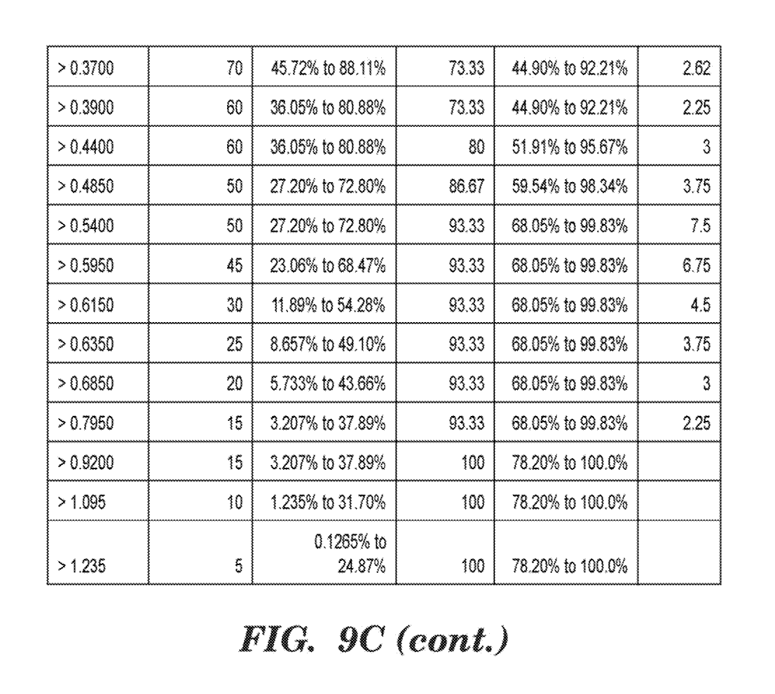

Lee et al., reported that clusterin inhibited fibril formation of TTR variants, and assessed if clusterin blocked TTR fibril-induced cytotoxicity. Lee et al., reported that clusterin decreased the proportion of sedimentable TTR fibrils formed from TTR variants (data not shown). Pellet fractions of TTR fibrils formed in the presence or absence of clusterin were added to SH-SY5Y cells and incubated for 2 days (FIG. 4). Lee et al., reported that clusterin significantly protected against neurotoxicity induced by fibrils of the TTR variants, and that clusterin inhibits amyloid fibril formation of TTR variants V30M and L55P by stabilizing TTR tetramers and thus suppresses amyloid fibril-induced cytotoxicity.

Previous studies have reported that clusterin can act as an extra-cellular molecular chaperone to affect the amyloid-forming process both in vivo and in vitro [10,11]. Clusterin is a well characterized extracellular chaperone and has been reported to inhibit amyloid formation by amyloid b peptide, lysozyme, apolipoprotein C-II, and a fragment of the prion protein [15-18]. Furthermore, clusterin is the only acid-activated extracellular molecular chaperone identified to date, showing enhanced binding to the stressed proteins under acidic conditions [19]. Therefore, Lee et al., reported that applying an acid-activated extracellular molecular chaperone to the inhibition of TTR amyloidosis may be useful because amyloid fibril formation of TTR may initiate in acidic environments of endosomes or lysosomes, leading to the partial denaturation of TTR proteins.

Thus, Lee et al. reported that an acid-activated extracellular molecular chaperone, clusterin affects TTR amyloidosis, and used GST-fusion proteins bound to Sepharose 4B as affinity matrix for clusterin binding experiments. Lee et al., used GST-fusion proteins bound to glutathione-Sepharose 4B as affinity matrix for clusterin binding experiments to determine the binding of clusterin to GST, wt-TTR and TTR variants appeared low in PBS, and reported the binding of clusterin to wt-TTR and TTR variants under acidic conditions (pH 4.4) was much greater than that in PBS (e.g. pH 7.6) during 12-24 h.

Although Lee et al., reported that the binding of clusterin to GST occurred at a low level, clusterin was reported to strongly interact with both wt-TTR and TTR variants V30M and L55P under acidic conditions and also inhibit amyloid fibril formation of TTR variants in vitro. In particular, Lee et al., reported that the extent of V30M TTR fibril formation in the presence of clusterin was comparable to the level of wt-TTR fibril formation. They also reported that inhibition of L55P TTR fibril formation by clusterin was less than expected when compared to its effect on V30M TTR amyloidosis, probably because L55P TTR variant is distinct from other TTR variants in that L55P TTR variant can easily dissociate into monomeric intermediates and form fibril precursors even under physiological conditions [24,25]. Lee et al., also reported that one mechanism of clusterin inhibition of TTR amyloidoses is through the stabilization of tetrameric structure of TTR, thereby reducing the amount of monomeric form available for amyloid fibril formation. They also reported that clusterin exhibited a decrease in chaperone activity as measure by its ability to stabilize tetrameric form of TTR variants over time, reporting that acidic conditions of less than 7.6 and pH5.0 are optimal, and pH levels below pH 5.0 may induce slow changes in the tertiary structure of clusterin which may decrease chaperone activity.

A number of studies have reported that axonal degeneration and neuronal loss are associated with extensive amyloid deposits commonly formed from wt-TTR and TTR variants [26]. Lee et al., report that clusterin effectively suppressed TTR fibril-induced neurotoxicity, and that V30M TTR fibril-induced neurotoxicity was completely blocked by clusterin, whereas L55P TTR fibril-mediated neurotoxicity was significantly but not completely inhibited by clusterin. They reported that the level of sedimentable L55P TTR fibrils in the presence of clusterin was higher than that of V30M TTR; and that the different amount of fibrils formed by each TTR variant likely accounts for their differing activities in their cytotoxicity assays.

In summary, Lee et al., reported that the extracellular molecular chaperone, clusterin is an effective inhibitor of FAP amyloidosis, and reported that Clusterin protein, e.g. human clusterin protein, substantially inhibits amyloid fibril formation of TTR variants V30M and L55P as well as amyloid fibril-induced neurotoxicity. Lee et al., discuss a chaperone-based therapy could be a useful therapeutic strategy for treating TTR amyloidosis and discuss that up-regulation of clusterin level or enhancement of its activity could be potential targets.

### Example 1

The inventors next investigated amyloid-infiltrated cardiac tissue for the presence of CLU and measured serum levels of CLU in patients with amyloidotic cardiomyopathy. Cardiac tissues containing congophilic deposits composed of either transthyretin (TTR) or immunoglobulin light chain (LC) from three patients with cardiomyopathy (CMP) were examined for the presence of CLU using immunohistochemical techniques. CLU staining co-localized with the intercellular myocardial amyloid in tissues from patients with familial transthyretin (ATTR), senile systemic (SSA), or immunoglobulin light chain (AL) amyloidosis. No CLU was found in control sections from non-amyloidotic heart tissue. The association of CLU with cardiac amyloid deposits was confirmed by immunogold electron microscopy.

Serum concentrations of CLU in patients with SSA, ATTR with CMP, AL with CMP, or AL with no CMP were measured by ELISA and compared to levels in age-matched controls. We found a significant decrease of serum CLU in SSA (p<0.005, n=35), ATTR-CMP (p<0.005, n=19), and AL-CMP (p<0.005, n=15) relative to the controls (n=25). Levels in AL with no CMP were comparable to the controls. Our data demonstrates that CLU is found associated with cardiac amyloid deposits and that serum levels of CLU are significantly lower in SSA and age-matched patients with cardiac amyloid disease due to ATTR or AL.

### Example 2

Cardiac Amyloid Deposits Contain CLU

The presence of amyloid deposits in autopsied cardiac specimens from three SSA, three ATTR, and three AL (kappa LC) cases were confirmed by histological treatment with Congo red; tissue from a nonamyloid heart transplant patient served as a control specimen. Light microscopic analysis of the Congo red-stained sections from all nine amyloid cardiac tissues revealed the green birefringence characteristic of amyloid deposits when viewed under polarized light (data not shown, as has been reported by Krijnen et al). No staining was evident in the control sections (data not shown).

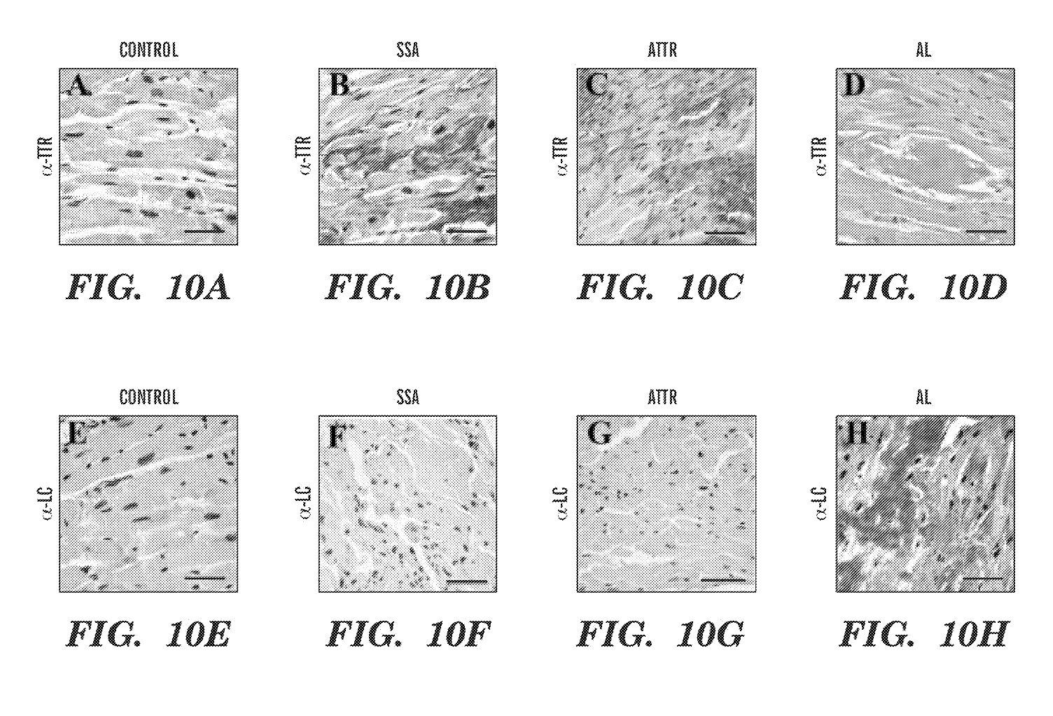

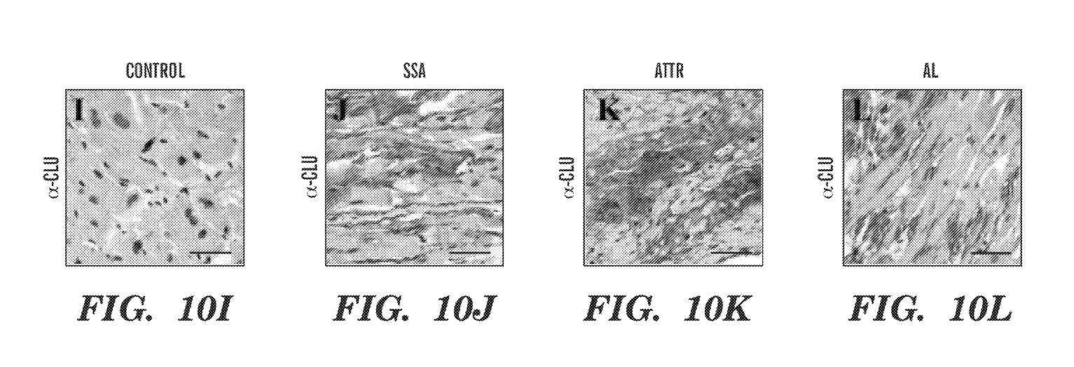

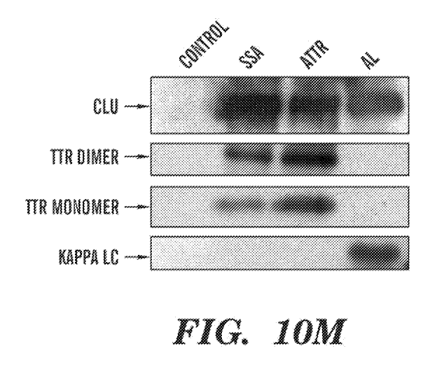

The presence of CLU in the cardiac amyloid deposits of patients with SSA, ATTR, or AL was initially investigated by immunohistochemistry (FIG. 10). The biochemical nature of the deposits, previously identified as amyloid with Congo red, was confirmed with the appropriate antibody treatment in serial sections from each of the nine tissues. TTR was identified in SSA and ATTR sections (FIG. 10B and FIG. 10C), and kappa LC was verified in the AL samples (FIG. 10H). The control sections were not immunoreactive to TTR or LC antibodies (FIG. 10A and FIG. 10E). In addition, there was no evidence of nonspecific primary antibody binding in the amyloid-laden tissues (FIG. 10D, FIG. 10F, and FIG. 10G) or nonspecific secondary antibody binding in the PIB controls (data not shown). Disruption of normal tissue architecture by the amyloid deposits, located adjacent to and surrounding cardiomyocytes, was evident. TTR and LC deposits exhibited pericellular staining patterns surrounding individual cardiomyocytes.

The immunohistochemical results for CLU indicated that the chaperone was present and highly abundant in all tissue sections that were positive for amyloid deposits (FIG. 10J, FIG. 10K, and FIG. 10L). Moreover, the pericellular staining pattern of CLU in each section was strikingly similar to that observed for the amyloid protein. In AL tissue, extracellular CLU was apparent; additionally, subtle intracellular staining indicated possible cardiomyocyte expression of CLU, as has been reported by Krijnen et al (FIG. 10L). Immunohistochemical results were consistent in all nine amyloid tissues that were analyzed; the results obtained on additional tissues (not shown in FIG. 10). Immunoblot analysis of protein extracts from the cardiac tissues confirmed the overabundance of the amyloid protein and presence of CLU in the SSA, ATTR, and AL samples compared with the control (FIG. 10M). Lanes containing the control sample (total protein extracted from the nonamyloid cardiac tissue) were loaded at protein concentrations equal to that of the amyloid samples in the individual analysis. Control lanes showed no evidence of amyloid proteins; a low level of CLU was observed.

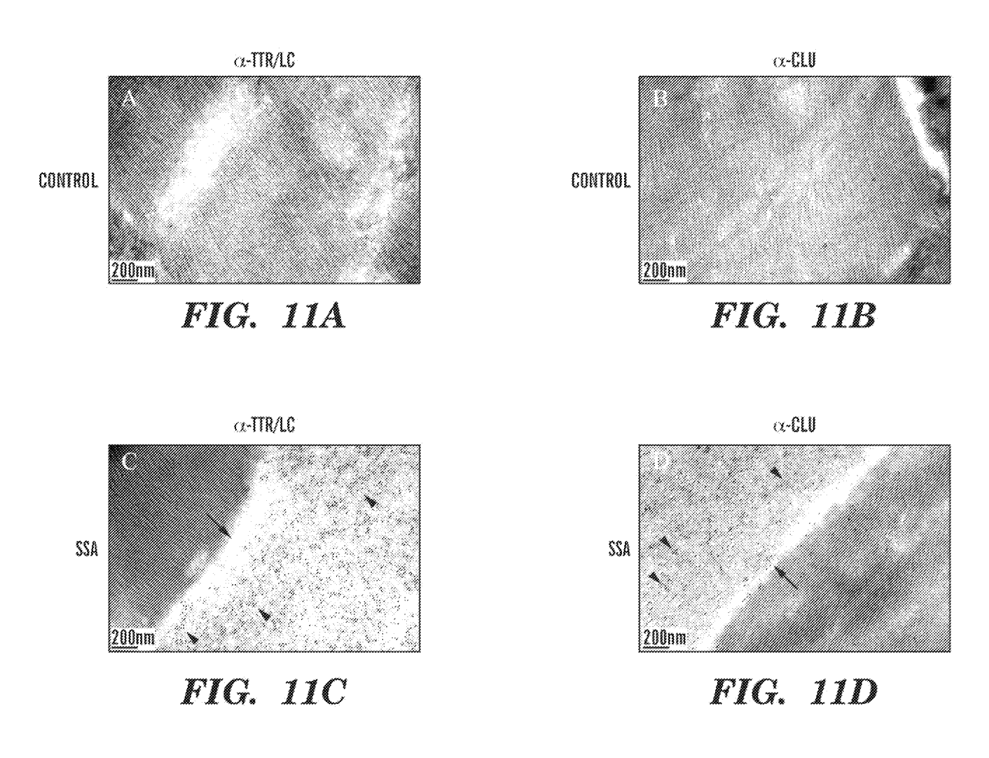

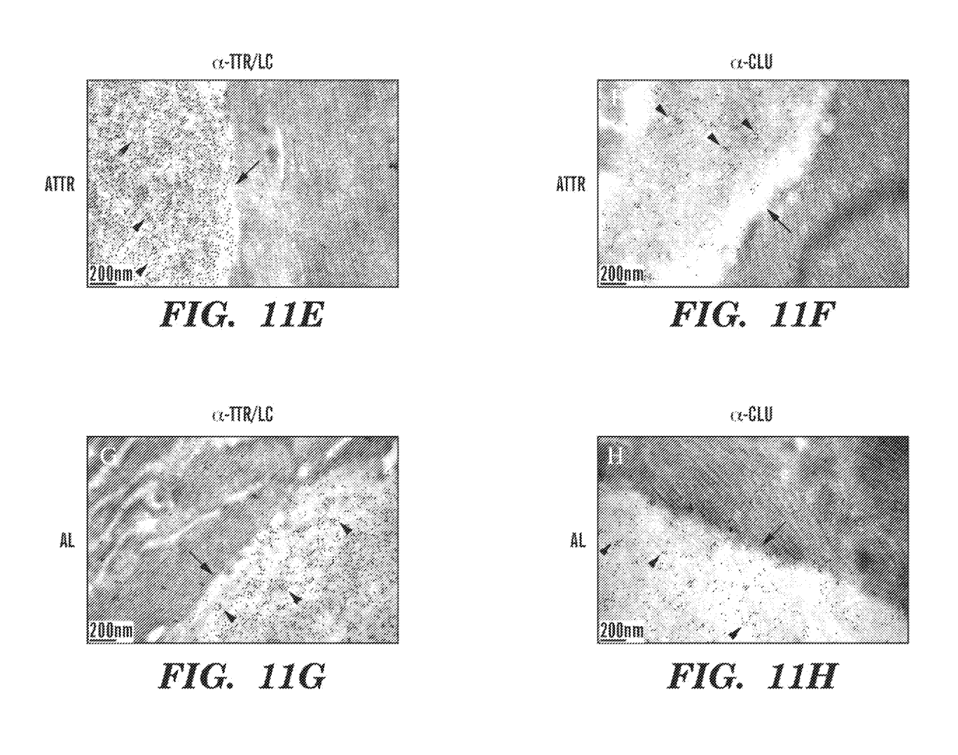

To confirm the presence of CLU in amyloid deposits composed of TTR or LC, immunogold electron microscopic analysis of the SSA, ATTR, and AL tissue specimens was performed, as shown in FIGS. 11A-H. Electron dense, 10 nm gold particles corresponding to TTR- (FIG. 11C, 11E) or LC- (FIG. 11G) and CLU- (FIG. 11D, 11F, 11H) specific antibodies are visible in areas where the fibrillar amyloid deposits are located, i.e. adjacent to the striated cardiomyocytes. Immunogold staining was negative in the control (non-amyloidic tissue) cardiac samples and in the amyloidosis patient samples where normal cellular and matrix components are visible; occasional randomly occurring gold particles observed in these regions were attributed to background staining. Furthermore, the amyloid tissues that were treated with PIB or normal rabbit serum (nonimmune) showed little or no staining (data not shown) which demonstrates that CLU is specifically associated with the amyloid fibrils in cardiac amyloid deposits of patients with SSA, ATTR, or AL.

### Example 3

Decreased CLU Levels in Amyloidotic CMP

The levels of CLU were measured in sera from patients with SSA, and ATTR or AL amyloidosis with and without CMP and compared to concentrations in healthy, age-matched controls. Included in this analysis were sera samples from a cohort of ischemic and non-ischemic CMP patients who did not have amyloidosis. Quantification was accomplished using a monoclonal capture ELISA system developed for this purpose. The mean±SEM values of serum CLU measured for the control group was 0.659±0.085 mg/mL. Concentrations of CLU were significantly lower in SSA (0.175±0.019 mg/mL, p<0.001) (FIG. 12A). In the ATTR and AL groups, the concentrations were varied, and a subanalysis of patients with and without CMP in these groups was performed. Serum CLU amounts in the ATTR and AL cohorts with no CMP were 0.743±0.038 mg/ml and 0.548±0.062 mg/ml, respectively. Compared with age-matched healthy controls, the ATTR-CMP (0.386±0.026 mg/ml) and AL-CMP (0.291±0.055 mg/ml) groups were significantly lower (P<0.001). Additionally, the ATTR-CMP and AL-CMP groups were significantly lower than either the ATTR no-CMP group or the AL no-CMP group (all P values <0.01). CLU levels were comparable in the AL and ATTR with no CMP groups, the nonamyloid patients with CMP, and the age-matched healthy controls. A comparison of groups with amyloidotic CMP (SSA, ATTR-CMP, AL-CMP) showed no significant difference.

CLU levels in the (a) amyloidosis patients with no cardiac involvement and (b) ischemic and non-ischemic CMP patients (Amyloid negative) were comparable to those of age-matched controls controls. SDS-PAGE and immunoblot analysis of representative serum samples from each group of patients and controls was consistent with the ELISA data; lower circulating levels of CLU was evident in the amyloid patient groups with cardiomyopathy relative to control groups (FIG. 12B).

### Example 4

Markers of Cardiac Remodeling

Serum BNP levels in the tested samples were retrospectively analyzed when available to confirm the presence of heart failure. BNP concentrations ≧100 pg/mL were present in all patients with CMP; this included patients with (ATTR, AL, SSA) or without amyloidosis (FIG. 12C). Mean BNP levels were highest in the AL-CMP (1321±468 pg/mL) group; mean BMP levels were comparable for ATTR-CMP (381±78 pg/mL) and SSA (373±56 pg/mL). In the patient groups with no cardiomyopathy BNP levels were in the normal range. Correlation analysis showed no significant association of BNP with CLU. (Spearman r=−0.2476, two-tailed P>0.05, n=37).

In patients with cardiac involvement, echocardiography was used to characterize left ventricular mass (LVM) and was correlated with serum CLU. The LVM in the amyloid cohorts were SSA=299.9±20.99 g (n=21), AL-CMP=231.5±15.93 g (n=19), and ATTR-CMP=228.4±16.32 g (n=19). The amyloid negative, CMP positive cohort showed the greatest LVM=331.3±21.78 g (n=15). LVM were significantly greater in SSA and amyloid negative, CMP positive cohorts compared to ATTR-CMP or AL-CMP cohorts (p<0.05). A correlation analysis of serum CLU with LVM was found to have a significant inverse relationship among all amyloid CMP sera (FIG. 12D) i.e the greater the LVM the lower the CLU levels (Pearson r=−0.2248, one-tailed P<0.05, n=59). In the nonamyloid with CMP group, there was no significant correlation between CLU and LVM.

### Example 5

Herein, the inventors demonstrate that CLU is present in cardiac tissue derived from patients with SSA, ATTR, and AL amyloidosis. Furthermore, it was demonstrated that the specific association of CLU with cardiac amyloid fibrillar deposits in these three different forms of systemic amyloid disease. The inventors demonstrate increased cardiac tissue CLU in both TTR and LC amyloid CMP, and also demonstrate that decreased circulating levels of CLU are associated with amyloidotic CMP in these diseases.

The circulating CLU may be the source of the CLU incorporated into amyloid deposits in the heart; or alternatively, a local upregulation of CLU in the heart may be associated with reduced production of CLU elsewhere. The inventors have demonstrated that the association of CLU with TTR and LC amyloid deposits is not entirely organ specific, and have identified CLU in LC deposits of the kidney by immunohistochemistry (data not shown). The reduced CLU levels in SSA and TTR and AL amyloidosis patient population analyzed herein demonstrates a strong associated of decreased CLU levels with amyloidotic CMP than amyloidosis alone, and does not occur with other forms of cardiomyopathy.

At the molecular level mechanisms to maintain a properly folded proteome, a process described as ‘proteostasis’, must be in place to prevent aberrant protein aggregation and disease in vivo. ^{15, 16} One such mechanism is due to extracellular chaperones with the capacity to bind circulating non-native protein conformations and inhibit their aggregation.^{9,17} As an abundant extracellular chaperone, CLU is emerging as an integral player in the pathogenesis of a multitude of protein misfolding diseases. CLU expression has been found to be elevated in a multitude of pathologies including AD, cancer, atherosclerosis, and myocardial infarction; CLU seems to play a role in response to tissue injury.¹¹ It has also been reported that CLU colocalizes with protein deposits including Aβ in AD, Down's syndrome, and inclusion body myositis; prion protein in Creutzfeldt-Jakob disease; Lewy bodies in α-synucleinopathies; and drusen in age-related macular degeneration.¹⁸⁻²⁰ Early studies of CLU identified Aβ in the cerebrospinal fluid of AD patients as a putative binding target for the protein.²¹ It was the first extracellular chaperone to be identified, and extensive investigations over the past decade have shown that CLU has chaperoning function similar to small heat shock proteins like αB-crystalline.^{8, 17, 18, 22-24}

The binding of CLU to exposed hydrophobic regions of misfolded proteins has been demonstrated in vitro for various amyloidogenic precursor proteins. Yerbury et al. demonstrated the ability of CLU to prevent amyloid formation of eight different amyloidogenic proteins through specific binding to prefibrillar amyloid structures; TTR and LC were not examined in these studies.¹⁸ In addition high molecular weight (HMW) aggregate formation of CLU-chaperone client complexes has been characterized in vitro with aggregates of an estimated stoichiometry of 1:2 (CLU:client).²⁴ Recently similar HMW aggregate formation was demonstrated using a stressed human plasma model in which albumin, ceruloplasmin, and fibrinogen were identified as major CLU-client proteins supporting the chaperoning role of CLU in maintaining a folded extracellular proteome.²³ CLU also has the capacity to bind to damaged fibrillar proteins like elastin in human photo-aged skin as well as to misfolded keratin.^{25, 26}

Interestingly, the ability of CLU to prevent amyloid formation changes in a concentration dependent manner such that it can actually promote fibrillogenesis under appropriate conditions. In particular, when the amyloidogenic substrate is present in a large molar excess, CLU appears to enhance fibrillogenesis and is subsequently incorporated into insoluble aggregates.¹⁸ Considering this concentration dependent biphasic effect, it has been posited that under extreme concentration conditions CLU can stabilize misfolded protein conformations, maintaining normally buried hydrophobic regions in an exposed form and augment fibril formation.¹⁷

Cardiac infiltration by amyloid fibrils results in a CMP with symptoms such as congestive heart failure, or arrhythmias. Prognosis is largely dependent on severity of cardiac involvement, but also on amyloid type and age at initial diagnosis.² In addition to mechanical disruption from deposited amyloid fibrils, progressive amyloidotic heart failure is also thought to be associated with aberrant matrix metalloproteinase (MMP)-mediated tissue remodeling and cardiac amyloid infiltration.³ MMP-9 levels have been shown to be elevated in endomyocardial biopsies from patients with SSA or AL compared to controls.²⁷ It has also been reported that MMP-9 can degrade amyloid fibrils composed of Aβ in situ and TTR an vitro; suggesting a possible clearance mechanism for the amyloid deposits.^{28, 29} The chaperoning capacity of CLU may play a pivotal role in these dynamic microenvironment processes associated with aberrant amyloid dependent tissue remodeling.

The present invention as disclosed herein is based on the discovery that significantly lower levels of circulating CLU is associated with amyloid deposition in the heart across three disease states, and demonstrates a unique difference in amyloid pathology that is dependent on organ involvement. The inventors have demonstrated that there is clearly something unique about the left ventricular mass (LVM) in amyloid and the inverse relationship with CLU, e.g., the greater the LVM, the lower the CLU levels in the serum. The low serum CLU concentrations observed in SSA may be due to a combination of prolonged subclinical cardiac amyloidosis, significant cardiac remodeling, and a perpetual state of fibril fragmentation and CLU sequestration from circulation.

In summary, the inventors have demonstrated that low levels of serum CLU in the pathogenesis of SSA, ATTR, and AL amyloidotic CMP. As demonstrated herein in the Examples, CLU is present in cardiac amyloid deposits composed of TTR and LC by immunohistochemical and immunogold techniques. In addition, serum concentrations of CLU determined by ELISA are significantly decreased in patients with amyloidotic CMP and there is an inverse relationship with LVM. Thus, it is demonstrated herein that low levels of CLU in the serum can serve as a biomarker for amyloidotic CMP.

### REFERENCES

The references cited herein and throughout the application are incorporated herein by reference.

REFERENCES of numbers enclosed by square brackets (e.g., [#]) in the representative Example and throughout the specification.
[1] S. J. Richardson, Cell and molecular biology of transthyretin and thyroid hormones, Int. Rev. Cytol. 258 (2007) 137-193.
[2] J. W. Kelly, The alternative conformations of amyloidogenic proteins and their multi-step assembly pathways, Curr. Opin. Struct. Biol. 8 (1998) 101-106.
[3] A. Quintas, M. J. Saraiva, R. M. Brito, The tetrameric protein transthyretin dissociates to a non-native monomer in solution. Anovel model for amyloidogenesis, J. Biol. Chem. 274 (1999) 32943-32949.
[4] Y. Sekijima, J. W. Kelly, S. Ikeda, Pathogenesis of and therapeutic strategies to ameliorate the transthyretin amyloidoses, Curr. Pharm. Des. 14 (2008) 3219-3230.
[5] X. Hou, M. I. Aguilar, D. H. Small, Transthyretin and familial amyloidotic polyneuropathy. Recent progress in understanding the molecular mechanism of neurodegeneration, FEBS J. 274 (2007) 1637-1650.
[6] Y. Ando, Liver transplantation and new therapeutic approaches for familial amyloidotic polyneuropathy (FAP), Med. Mol. Morphol. 38 (2005) 142-154.
[7] K. Tojo, Y. Sekijima, J. W. Kelly, S. Ikeda, Diflunisal stabilizes familial amyloid polyneuropathy-associated transthyretin variant tetramers in serum against dissociation required for amyloidogenesis, Neurosci. Res. 56 (2006) 441-449.
[8]. Cardoso, G. Merlini, M. J. Saraiva, 40-iodo-40-deoxydoxorubicin and tetracyclines disrupt transthyretin amyloid fibrils in vitro producing noncytotoxic species: screening for TTR fibril disrupters, FASEB J. 17 (2003) 803-809.
[9] T. Mairal, J. Nieto, M. Pinto, M. R. Almeida, L. Gales, A. Ballesteros, J. Barluenga, J. J. Perez, J. T. Vazquez, N. B. Centeno, M. J. Saraiva, A. M. Damas, A. Planas, G. Arsequell, G. Valencia, Iodine atoms: a new molecular feature for the design of potent transthyretin fibrillogenesis inhibitors, PLoS ONE 4 (2009) e4124.
[10] M. R. Wilson, J. J. Yerbury, S. Poon, Potential roles of abundant extracellular chaperones in the control of amyloid formation and toxicity, Mol. Biosyst. 4 (2008) 42-52.
[11] J. J. Yerbury, E. M. Stewart, A. R. Wyatt, M. R. Wilson, Quality control of protein folding in extracellular space, EMBO Rep. 6 (2005) 1131-1136.
[12] S. E. Jones, C. Jomary, Clusterin, Int. J. Biochem. Cell Biol. 34 (2002) 427-431.
[13] S. Poon, S. B. Easterbrook-Smith, M. S. Rybchyn, J. A. Carver, M. R. Wilson, Clusterin is an ATP-independent chaperone with very broad substrate specificity that stabilizes stressed proteins in a folding-competent state, Biochemistry 39 (2000) 15953-15960.
[14] J. A. Carver, A. Rekas, D. C. Thorn, M. R. Wilson, Small heat-shock proteins and clusterin: intra- and extracellular molecular chaperones with a common mechanism of action and function?, IUBMB Life 55 (2003) 661-668
[15] J. J. Yerbury, S. Poon, S. Meehan, B. Thompson, J. R. Kumita, C. M. Dobson, M. R. Wilson, The extracellular chaperone clusterin influences amyloid formation and toxicity by interacting with prefibrillar structures, FASEB J. 21 (2007) 2312-2322.
[16] J. R. Kumita, S. Poon, G. L. Caddy, C. L. Hagan, M. Dumoulin, J. J. Yerbury, E. M. Stewart, C. V. Robinson, M. R. Wilson, C. M. Dobson, The extracellular chaperone clusterin potently inhibits human lysozyme amyloid formation by interacting with prefibrillar species, J. Mol. Biol. 369 (2007) 157-167.
[17] T. Oda, P. Wals, H. H. Osterburg, S. A. Johnson, G. M. Pasinetti, T. E. Morgan, I. Rozovsky, W. B. Stine, S. W. Snyder, T. F. Holzman, et al., Clusterin (apoJ) alters the aggregation of amyloid beta-peptide (Abeta 1-42) and forms slowly sedimenting A beta complexes that cause oxidative stress, Exp. Neurol. 136 (1995) 22-31.
[18] D. M. Hatters, M. R. Wilson, S. B. Easterbrook-Smith, G. J. Howlett, Suppression of apolipoprotein C-II amyloid formation by the extracellular chaperone, clusterin, Eur. J. Biochem. 269 (2002) 2789-2794.
[19] S. Poon, M. S. Rybchyn, S. B. Easterbrook-Smith, J. A. Carver, G. J. Pankhurst, M. R. Wilson, Mildly acidic pH activates the extracellular molecular chaperone clusterin, J. Biol. Chem. 277 (2002) 39532-39540.
[20] W. Colon, J. W. Kelly, Partial denaturation of transthyretin is sufficient for amyloid fibril formation in vitro, Biochemistry 31 (1992) 8654-8660.
[21] J. W. Kelly, W. Colon, Z. Lai, H. A. Lashuel, J. McCulloch, S. L. McCutchen, G. J. Miroy, S. A. Peterson, Transthyretin quaternary and tertiary structural changes facilitate misassembly into amyloid, Adv. Protein Chem. 50 (1997) 161-181.
[22] H. A. Lashuel, Z. Lai, J. W. Kelly, Characterization of the transthyretin acid denaturation pathways by analytical ultracentrifugation: implications for wild-type, V30M, and L55P amyloid fibril formation, Biochemistry 11 (1998) 17851-17864.
[23] S. L. Adamski-Werner, S. K. Palaninathan, J. C. Sacchettini, J. W. Kelly, Diflunisal analogues stabilize the native state of transthyretin, potent inhibition of amyloidogenesis, J. Med. Chem. 47 (2004) 355-374.
[24] H. A. Lashuel, C. Wurth, L. Woo, J. W. Kelly, The most pathogenic transthyretin variant, L55P, forms amyloid fibrils under acidic conditions and protofilaments under physiological conditions, Biochemistry 38 (1999) 13560-13573.
[25] M. Yang, M. Lei, S. Huo, Why is Leu55→Pro55 transthyretin variant the most amyloidogenic: insights from molecular dynamics simulations of transthyretin monomers, Protein Sci. 12 (2003) 1222-1231.
[26] M. M. Sousa, M. J. Saraiva, Neurodegeneration in familial amyloid polyneuropathy: from pathology to molecular signaling, Prog. Neurobiol. 71 (2003) 385-400.

REFERENCES of numbers in superscript (e.g., reference^{#}) in EXAMPLES 2-4 and throughout the specification.
1. Lavatelli F, Perlman D H, Spencer B, Prokaeva T, McComb M E, Therberge R, Connors L H, Bellotti V, Seldin D C, Merlini G, Skinner M, Costello C E: Amyloidogenic and Associated Proteins in Systemic Amyloidosis Proteome of Adipose Tissue, Mol. Cell Proteomics 2008, 7:1570-1583
2. Falk R H, Dubrey S W: Amyloid Heart Disease, Progress in Cardiovascular Diseases 2010, 52:347-361
3. Shah K B, Inoue Y, Mehra M R: Amyloidosis and the Heart: A Comprehensive Review, Arch Intern Med. 2006, 166:1805-1813
4. Sawyer D B, Skinner M: Cardiac Amyloidosis: Shifting Our Impressions to Hopeful, Curr Heart Fail Rep. 2006, 3:64-71
5. Hassan W, Al-Sergani H, Mourad W, Tabba R: Amyloid Heart Disease. New Frontiers and Insights in Pathophysiology, Diagnosis, and Managment, Texas Heart Institute Journal 2005, 32:178-184
6. Stefani M, Dobson C M: Protein aggregation and aggregate toxicity: new insights into protein folding, misfolding diseases and biological evolution, J Mol Med. 2003, 81:678-699.
7. Bailey R W, Dunker A K, Brown C J, Garner E C, Griswold M D: Clusterin, a Binding Protein with a Molten Globule-like Regionâ [symbol] Biochemistry 2001, 40:11828-11840
8. Humphreys D T, Carver J A, Easterbrook-Smith S B, Wilson M R: Clusterin Has Chaperone-like Activity Similar to That of Small Heat Shock Proteins, J Biol Chem. 1999, 274:6875-6881
9. Nuutinen T, Suuronen T, Kauppinen A, Salminen A: Clusterin: A forgotten player in Alzheimer's disease, Brain Res. Rev. 2009, 61:89-104
10. Aronow B J, Lund S D, Brown T L, Harmony J A, Witte D P: Apolipoprotein J expression at fluid-tissue interfaces: potential role in barrier cytoprotection, PNAS 1993, 90:725-729.
11. Rosenberg M E, Silkensen J: Clusterin: Physiologic and pathophysiologic considerations, Int. J. Biochem. Cell Biol. 1995, 27:633-645
12. Loison F, Debure L, Nizard P, leÂ goff P, Michel D, leÂ drÃ©an Y: Up-regulation of the clusterin gene after proteotoxic stress: implication of HSF1-HSF2 heterocomplexes, Biochem J. 2006, 395:223-231
13. Swertfeger D, Witte D, Stuart W, Rockman H, Harmony J: Apolipoprotein J/clusterin induction in myocarditis: A localized response gene to myocardial injury, Am J Pathol. 1996, 148:1971-1983
14. Devereux R B, Alonso D R, Lutas E M, Gottlieb G J, Campo E, Sachs I, Reichek N: Echocardiographic assessment of left ventricular hypertrophy: comparison to necropsy findings, Am J Cardiol. 1986, 57:450-458.
15. Powers E T, Morimoto R I, Dillin A, Kelly J W, Balch W E: Biological and Chemical Approaches to Diseases of Proteostasis Deficiency, Annu Rev Biochem. 2009, 78:959-991
16. Balch W E, Morimoto R I, Dillin A, Kelly J W: Adapting Proteostasis for Disease Intervention, Science 2008, 319:916-919
17. Wilson M R, Yerbury J J, Poon S: Potential roles of abundant extracellular chaperones in the control of amyloid formation and toxicity, Molecular BioSystems 2008, 4:42-52
18. Yerbury J J, Poon S, Meehan S, Thompson B, Kumita J R, Dobson C M, Wilson M R: The extracellular chaperone clusterin influences amyloid formation and toxicity by interacting with prefibrillar structures, FASEB J. 2007, 21:2312-2322
19. Freixes M, Puig B, Rodriguez A, Torrejón-Escribano B, Blanco R, Ferrer I: Clusterin solubility and aggregation in Creutzfeldt-Jakob disease, Acta Neuropathologica 2004, 108:295-301
20. Sasaki K, Doh-ura K, Wakisaka Y, Iwaki T: Clusterin/apolipoprotein J is associated with cortical Lewy bodies: immunohistochemical study in cases with a-synucleinopathies, Acta Neuropathologica 2002, 104:225-230
21. J Ghiso, E Matsubara, A Koudinov, N H Choi-Miura, M Tomita, T Wisniewski, Frangione. B: The cerebrospinal-fluid soluble form of Alzheimer's amyloid beta is complexed to SP-40,40 (apolipoprotein J), an inhibitor of the complement membrane-attack complex, Biochem J. 1993, 293:27-30.
22. Wilson M R, Easterbrook-Smith S B: Clusterin is a secreted mammalian chaperone, Trends Biochem. Sci. 2000, 25:95-98
23. Wyatt A R, Wilson M R: Identification of human plasma proteins as major clients for the extracellular chaperone clusterin, J Biol Chem. 2010, 285:3532-3539
24. Wyatt A R, Yerbury J J, Wilson M R: Structural Characterization of Clusterin-Chaperone Client Protein Complexes, J Biol Chem. 2009, 284:21920-21927
25. Janig E, Haslbeck M, Aigelsreiter A, Braun N, Unterthor D, Wolf P, Khaskhely N M, Buchner J, Denk H, Zatloukal K: Clusterin Associates with Altered Elastic Fibers in Human Photoaged Skin and Prevents Elastin from Ultraviolet-Induced Aggregation in Vitro, Am J. Pathol. 2007, 171:1474-1482
26. Janig E, Stumptner C, Fuchsbichler A, Denk H, K. Z: Interaction of stress proteins with misfolded keratins, Eur J Cell Biol. 2005, 84:329-339
27. Biolo A, Ramamurthy S, Connors L H, O'Hara C J, Meier-Ewert H K, Soo Hoo P T, Sawyer D B, Seldin D S, Sam F: Matrix Metalloproteinases and Their Tissue Inhibitors in Cardiac Amyloidosis: Relationship to Structural, Functional Myocardial Changes and to Light Chain Amyloid Deposition, Circ Heart Fail. 2008, 1:249-257
28. Yan P, Hu X, Song H, Yin K, Bateman R J, Cirrito J R, Xiao Q, Hsu F F, Turk J W, Xu J, Hsu C Y, Holtzman D M, Lee J-M: Matrix Metalloproteinase-9 Degrades Amyloid-β Fibrils in Vitro and Compact Plaques in Situ, J Biol Chem. 2006, 281:24566-24574
29. Sousa M M, Barbas do Amaral J, Guimaraes A, Saraiva M J: Up-regulation of the extracellular matrix remodeling genes, biglycan, neutrophil gelatinase-associated lipocalin and matrix metalloproteinase-9 in familial amyloid polyneuropathy, FASEB J. 2004, 19:124-126

1. An assay comprising: testing for clusterin levels in a biological sample to determine if a subject has a likelihood of cardiac amyloid deposits by using a protein-binding molecule which specifically binds to clusterin and a reagent which can detect when the protein-binding molecule is specifically bound to clusterin, and determining if clusterin is present in the sample, where the level of clusterin indicates the subject is likely to have cardiac amyloid deposits.

2. The assay of claim 1, further comprising:
performing the assay to detect the level of clusterin in a biological sample obtained from a subject,
measuring the amount of clusterin protein in the biological sample bound to the protein-binding molecule; and
identifying a subject with cardiac amyloid deposits based on the amount of clusterin protein in the biological sample, wherein the amount of clusterin protein in a biological sample from a subject with cardiac amyloid deposits is lower by a statistically significant amount than the amount of clusterin protein in a biological sample from a subject who is a healthy subject or a subject not having a disease or disorder associated with amyloidosis.

3. The assay of claim 1, further comprising: comparing the amount of clusterin protein in the biological sample from the subject with the amount of clusterin protein in a biological tissue of a subject who is a healthy subject or a subject not having a disease or disorder associated with amyloidosis, wherein a decrease in the level of the clusterin protein in the biological sample by more than 40% as compared to a subject who does not have cardiac amyloid deposits is indicative of the subject being at risk of having cardiac amyloid deposits.

4. (canceled)

5. The assay of any of claim 1, wherein the biological sample is a blood sample, plasma sample or a serum sample.

6. (canceled)

7. The assay of claim 1, wherein a protein binding molecule is an antibody, or an antibody fragment or a ligand binding partner of clusterin, or functional fragments thereof.

8. The assay of claim 2, wherein the level of clusterin protein is measured by immunoassay or ELISA.

9. The assay of claim 2, wherein the level of clusterin protein is measured by any one of the group consisting of ELISA, radioimmunoassay (RIA), immunoradiometric assay (IRMA), immunoenzymometric assay (IEMA), immunohistochemistry, western blotting, mass spectroscopy, SELDI/TOF, liquid chromatography, nuclear magnetic resonance spectroscopy (MS/MS).

10. The assay of claim 1, wherein the subject is has, or is at risk of having a disease or disorder associated with amyloidosis.

11. The assay of claim 10, wherein a disease or disorder associated with amyloidosis is a disease or disorder associated with transthyretin (TTR) amyloidosis or immunoglobulin light chain (LC) amyloidosis.

12. The assay of claim 11, wherein a disease or disorder associated with transthyretin (TTR) amyloidosis is selected from the group consisting of: senile systemic amyloidosis (SAA), senile cardiac amyloidosis (SCA), familial amyloid neuropathy (FAP), familial amyloid cardiomyopathy (FAC), familial central nervous system amyloidosis.

13. (canceled)

14. (canceled)

15. The assay of claim 2, wherein a level of the clusterin protein between 0.5 mg/mL and 0.1 mg/ml in the biological sample is indicative of the subject being at risk of having cardiac amyloid deposits.

16. (canceled)

17. (canceled)

18. (canceled)

19. (canceled)

20. The assay of claim 2, wherein the subject identified as having cardiac amyloid deposits is at risk of having, or has congestive heart failure, arrhythmias or progressive amyloid heart failure.

21. The assay of claim 2, further comprising administering to the subject identified to be at risk of having amyloid deposits a composition comprising a clusterin protein or a functional fragment having clusterin activity, or an agonist of clusterin activity.

22. The assay of claim 21, wherein the clusterin protein or a functional fragment having clusterin activity or agonist of clusterin activity decreases the amount of TTR amyloid fibrils in the subject.

23. (canceled)

24. The assay of claim 21, wherein clusterin protein is SEQ ID NO: 1 or a functional fragment thereof.

25. The assay of claim 21, wherein the agonist of clusterin activity is a functional isoform, a functional mutein, a functional fused protein, a functional derivative, a functional variant or a functional fragment of clusterin protein.

26. The assay of claim 21, wherein the functional variant or functional mutein is a variant or mutein of SEQ ID NO: 1 which has at least 80%, or at least 85%, or at least 90%, or at least 95% homology with SEQ ID NO: 1.

27. (canceled)

28. The assay of claim 21, wherein the composition comprising clusterin is at an acidic pH of less than pH 6.0.

29. (canceled)

30. The assay of claim 28, wherein the acidic pH is between pH 6.0 and pH 4.5.

31. A system comprising:
a determination module configured to receive a biological sample, measure levels of clusterin protein in the biological sample and to output information of the level of clusterin protein in the biological sample;
a storage device configured to store clusterin level output information from the determination module;
a comparison module adapted to receive input from the storage device and compare the data stored on the storage device with at least one reference clusterin level data, wherein if the reference clusterin level data is 1.5-fold or more higher than the input clusterin protein level information, the comparison module provides information to an output module that the biological sample is associated with a subject that deviates from the reference clusterin protein level;
an output module for displaying the information to the user.

32. An assay for determining the increased likelihood of a subject having cardiac amyloid deposits, comprising:
contacting a protein-binding molecule which specifically binds to clusterin with a biological sample obtained from the subject,
allowing the protein-binding molecule to bind to clusterin
quantifiably detecting bound clusterin in the biological sample to obtain a level of clusterin in the biological sample,
comparing the detected level of clusterin in the biological sample with a reference value for clusterin levels, wherein the reference value is the level of clusterin in a comparable biological sample from a subject without cardiac amyloid deposits, the level of clusterin level relative to the reference value being indicative of the presence of cardiac amyloid deposits, wherein a clusterin level from the biological sample that is lower than the reference value by a statistically significant degree is indicative of the presence of cardiac amyloid deposits, or
comparing the detected level of clusterin in the biological sample with a reference value for clusterin levels in a comparable biological sample from a subject in which cardiac amyloid deposits are known to be present, the level of clusterin level relative to the reference value being indicative of the presence of cardiac amyloid deposits, wherein a clusterin level from the biological sample that deviates from the reference value by a statistically significant degree is indicative of the absence of cardiac amyloid deposits.

33. The assay of claim 32, wherein the reference value of clusterin without cardiac amyloid deposits is the level of clusterin present in the same type of biological sample obtained from a healthy subject or a subject not having a disease or disorder associated with amyloidosis.

34. An assay to screen a subject to determine the increased likelihood of the presence of cardiac amyloid deposits, comprising: determining a level of clusterin in a biological sample from the subject, and identifying the subject as having an increased likelihood of cardiac amyloid deposits if the level of clusterin is lower than 0.5 mg/mL; or identifying the subject as having a decreased likelihood of cardiac amyloid deposits if the level of clusterin is higher than 0.5 mg/ml.
